(19) United States
(12) Patent Application Publication
(10) Pub. No.: US 2011/0020370 A1
(43) Pub. Date: Jan. 27, 2011

(54) SLC7A5 DIRECTED DIAGNOSTICS AND THERAPEUTICS FOR NEOPLASTIC DISEASE

(76) Inventors: Elias GEORGES, Laval (CA); Anne-Marie BONNEAU, Laval (CA)

Correspondence Address: WILMERHALE/BOSTON, 60 STATE STREET, BOSTON, MA, 02109, US

(21) Appl. No.: 12/361,790

(22) Filed: Jan. 29, 2009

Related U.S. Application Data
(60) Provisional application No. 61/121,604, filed on Dec. 11, 2008.

(51) Int. Cl. A61K 39/395; C12Q 1/68; C07K 16/30; A61K 39/00; A61P 25/00

(52) U.S. Cl. 424/174.1; 435/6; 530/389.7; 424/277.1; 424/185.1

(57) ABSTRACT

Disclosed are methods for diagnosing cancer in a test cell sample or fluid sample by detecting an increase in the level of expression of SLC7A5 in the test cell sample or fluid sample as compared to the level of expression of SLC7A5 in a control cell sample or fluid sample isolated from a normal subject.

### CROSS REFERENCE TO RELATED APPLICATIONS

This application claims the benefit of U.S. Provisional Patent Application Ser. No. 61/121,604 of Elias Georges, et al. entitled “SLC7A5 Directed Diagnostics and Therapeutics for Neoplastic Disease,” filed Dec. 11, 2008. The entirety of the provisional patent application is incorporated herein by reference.

### FIELD OF THE INVENTION

The present disclosure relates generally to the field of medicine. More specifically, the disclosure pertains to methods and kits for detecting the development of, and treating, cancer in a subject.

### BACKGROUND OF THE INVENTION

Cancer is one of the most deadly illnesses in the world. It accounts for nearly 600,000 deaths annually in the United States, and costs billions of dollars for those who suffer from the disease. This disease is in fact a diverse group of disorders, which can originate in almost any tissue of the body. In addition, cancers may be generated by multiple mechanisms including pathogenic infections, mutations, and environmental insults. The variety of cancer types and mechanisms of tumorigenesis add to the difficulty associated with treating a tumor, increasing the risk posed by the cancer to the patient's life and well-being.

Cancers manifest abnormal growth and the ability to move from an original site of growth to other tissues in the body (“metastasis”), unlike most non-cancerous cells. These clinical manifestations are therefore used to diagnose cancer because they are applicable to all cancers. Additionally, a cancer diagnosis is made based on identifying cancer cells by their gross pathology through histological and microscopic inspection of the cells.

Although the gross pathology of the cells can provide accurate diagnoses of the cells, the techniques used for such analysis are hampered by the time necessary to process the tissues and the skill of the technician analyzing the samples. These methodologies can lead to unnecessary delay in treating a growing tumor, thereby increasing the likelihood that a benign tumor will acquire metastatic characteristics. It is thus necessary to accurately diagnose potentially cancerous growths in early stages to avoid the development of a potentially life threatening illness.

One potential method of increasing the speed and accuracy of cancer diagnoses is the examination of genes as markers for neoplastic potential. Recent advances in molecular biology have identified genes involved in cell cycle control, apoptosis, and metabolic regulation (see, e.g., Isoldi et al. (2005) Mini Rev. Med. Chem. 5(7): 685-95). Mutations in many of these genes have also been shown to increase the likelihood that a normal cell will progress to a malignant state (see, e.g., Soejima et al. (2005) Biochem. Cell Biol. 83(4): 429-37). Many mutations can affect the levels of expression of certain genes in the neoplastic cells as compared to normal cells.

There remains a need to identify an accurate and rapid means for diagnosing cancer in patients, and to treat such cancer. Treatment efficacy would be improved by more efficient diagnoses of fluid (e.g., blood) or tissue samples. Furthermore, rapid diagnoses of cancerous tissues or blood samples from patients may allow clinicians to treat potential tumors prior to the metastasis of the cancer to other tissues of the body. Also, a test that does not rely upon a particular technician's skill at identifying abnormal histological characteristics would improve the reliability of cancer diagnoses. There is, therefore, a need for new methods of diagnoses for cancer that are accurate, fast, and relatively easy to interpret. In addition, such tests are useful to follow the response of patients to cancer treatment.

### SUMMARY OF THE INVENTION

The present disclosure is based in part upon the discovery that differential expression of SLC7A5 (solute carrier family 7 (cationic amino acid transporter, y+ system), member 5; or “SLC7A5”) at the protein and RNA levels occurs when a cell progresses to a neoplastic state. These expression patterns are therefore diagnostic for the presence of cancer in a cell sample. This discovery has been exploited to provide an invention that uses such patterns of expression to diagnose the presence of neoplastic cells in the test sample (cell sample or fluid sample, where the protein is secreted or released in circulation), and to treat such neoplasms in a patient. The SLC7A5 may be found as full length protein and/or peptides or fragments of SLC7A5. Similarly, a test sample for diagnosis may contain the SLC7A5 RNA or modified nucleotide fragments of this gene.

Accordingly, in one aspect, the disclosure provides a method of detecting a neoplasm comprising: a) obtaining a potentially neoplastic test sample and a corresponding non-neoplastic control sample; b) detecting a level of SLC7A5 expression in the test sample and in the control sample; and c) comparing the level of SLC7A5 expression in the test sample to the level of SLC7A5 expression in the control sample. The test sample is neoplastic if the level of SLC7A5 expression in the test sample is detectably greater than the level of SLC7A5 expression in the control sample.

In some embodiments, the level of expression of SLC7A5 protein is detected by contacting the test sample and the control sample with a SLC7A5-specific protein binding agent selected from the group consisting of an anti-SLC7A5 antibody, SLC7A5-binding portions of an antibody, SLC7A5-specific ligands, SLC7A5-specific aptamers, and SLC7A5 inhibitors. In certain embodiments, SLC7A5-specific binding agent bound to SLC7A5 protein further comprises a detectable label. In particular embodiments, the detectable label is selected from the group consisting of an immunofluorescent label, a radiolabel, and a chemiluminescent label.

In some embodiments, the SLC7A5-specific protein binding agent is immobilized on a solid support.

In other embodiments, SLC7A5 expression is detected by detecting the level of expression of SLC7A5 RNA by contacting the test sample and the control sample with a SLC7A5 RNA-specific nucleic acid binding agent and determining how much of the nucleic acid binding agent is hybridized to SLC7A5 RNA in the test sample and in the control sample. In some embodiments, the level of nucleic acid binding agent hybridized to SLC7A5 RNA is detected using a detectable label operably linked to the binding agent. In particular embodiments, the label is selected from the group consisting of an immunofluorescent label, a radiolabel, and a chemiluminescent label. In certain embodiments, the nucleic acid binding agent is immobilized on a solid support.

In some embodiments, the level of expression of SLC7A5 in the test sample is at least 1.5 times greater, at least 2 times greater, at least 4 times greater, at least 6 times greater, at least 8 times greater, at least 10 times greater, or at least 20 times greater than the level of expression of SLC7A5 in the control sample. In certain embodiments, the test sample is isolated from a patient suffering from ovarian cancer, breast cancer, colon cancer, lung cancer, melanoma, sarcoma, or leukemia, and in some embodiments, the cancer is a metastasized cancer.

In particular embodiments, neoplastic test sample and the control samples are cell samples of the same lineage. In certain embodiments, a cytoplasmic fraction is isolated from the test cell sample and from the control cell sample, and then the level of expression of SLC7A5 in each of these cytoplasmic fractions is detected separately

In other embodiments, the test sample and the control samples are fluid samples. In certain embodiments, the fluid samples are blood, serum, urine, seminal fluid, lacrimal secretions, sebaceous gland secretions, tears, or vaginal secretions. In a particular embodiment, the fluid sample is a serum sample. In some embodiments, the level of SLC7A5 protein expression is determined by measuring the level of anti-SLC7A5 antibody in the test fluid sample and in the control fluid sample. In certain embodiments, the level of expression of anti-SLC7A5 antibody is detected with an anti-SLC7A5 antibody-specific antibody, or anti-SLC7A5 antibody-specific antibody fragment thereof. In some embodiments, the anti-SLC7A5 antibody-specific antibody, or anti-SLC7A5 antibody-specific binding fragments thereof, are operably linked to a detectable label.

In another aspect, the disclosure provides a method for detecting a neoplasm comprising: a) obtaining a potentially neoplastic test sample and a non-neoplastic control sample; b) detecting a level of SLC7A5 expression in the test sample and in the control sample; c) detecting a level of expression of at least one of UHRF1, TRIM59, TTK, and/or KIF20A; and d) comparing the level of SLC7A5 expression and the level of expression of at least one of TRIM59, TTK, UHRF1 and/or KIF20A in the test sample to the level of SLC7A5 expression and the level of expression of the at least one of TRIM59, TTK, UHRF1 and/or KIF20A in the control sample. The test sample is neoplastic if the levels of expression of SLC7A5 and the at least one of TRIM59, TTK, UHRF1 and/or KIF20 in the test sample are detectably greater than the levels of expression of SLC7A5 and the at least one of TRIM59, TTK, UHRF1 and/or KIF20A in the control sample. In some embodiments, besides detecting and comparing the levels of SLC7A5 expression, the level of expression of at least TRIM59 is also detected and compared to the level of expression in test and control samples.

In some embodiments, the level of SLC7A5 expression is detected by contacting the test sample and the control sample with a SLC7A5-specific protein binding agent selected from the group consisting of an SLC7A5-specific antibody, SLC7A5-specific binding portions of an antibody, a SLC7A5-specific ligand, a SLC7A5-specific aptamer, and an SLC7A5 inhibitor. In certain embodiments, the SLC7A5-specific protein binding agent is immobilized on a solid support.

In other embodiments, the level of expression of SLC7A5 in the test and control samples is measured by measuring the level of SLC7A5 RNA and the level of at least one of TRIM59 RNA, TTK RNA, UHRF1 RNA, and/or KIF20A RNA in the test and control samples. In some embodiments, the level of expression of SLC7A5 RNA and the level of expression of at least one of TRIM59 RNA, TTK RNA, UHRF1 RNA, and/or KIF20A RNA are detected by contacting the test sample and the control sample with an TTK-specific nucleic acid binding agent and with at least one of a TRIM59-specific nucleic acid binding agent, a SLC7A5-specific nucleic binding agent, a UHRF1-specific nucleic acid binding agent, and a KIF20A-specific nucleic acid binding agent.

In some embodiments, the levels of expression of UHRF1, TTK, SLC7A5, TRIM59 and/or KIF20 in the test sample are at least about 1.5, 2, 5, 10, or 20 times greater than the level of expression of UHRF1, TTK, SLC7A5, TRIM59, and/or KIF20 in the control sample.

In particular embodiments, detecting the level of expression of SLC7A5 and the level of expression of at least one of TRIM59, TTK, UHRF1 and/or KIF20A comprises isolating a cytoplasmic fraction from the test cell sample and from the control cell sample, and then detecting the levels of expression of SLC7A5 and at least one of TRIM59, TTK, UHRF1 and/or KIF20A in each of these cytoplasmic fractions.

In some embodiments, the test and control samples are fluid samples, and in certain embodiments, the level of expression of SLC7A5 is measured by detecting a level of anti-SLC7A5 antibody in a test fluid sample and in a control fluid sample.

In certain embodiments, the test sample is isolated from a tissue of a patient suffering from ovarian cancer, breast cancer, lung cancer, sarcoma, melanoma, or leukemia. In particular embodiments, the cancer has metastasized.

In yet another aspect, the disclosure provides a kit for diagnosing or detecting neoplasia. The kit comprises: a) a first probe specific for the detection of SLC7A5; and b) a second probe specific for the detection of a neoplasia marker selected from the group consisting of TRIM59, TTK, UHRF1, KIF20A, and combinations thereof.

In some embodiments, the probe for detecting SLC7A5 is an anti-SLC7A5-specific antibody or an SLC7A5-specific binding fragment thereof, a SLC7A5-specific aptamer, or SLC7A5-specific ligand.

In some embodiments, the second probe is selected from the group consisting of a TRIM59-specific antibody, a TRIM59-specific binding portion of TRIM59 antibody, a TRIM59-specific ligand, a TRIM59-specific aptamer, a SLC7A5-specific antibody, a SLC7A5-specific binding portion of a SLC7A5-specific antibody, a SLC7A5-specific ligand, a SLC7A5-specific aptamer, a UHRF1-specific antibody, a UHRF1-specific binding portion of a UHRF1-specific antibody, a UHRF1-specific ligand, a UHRF1-specific aptamer, a KIF20A-specific binding portion of a KIF20A-specific antibody, a KIF20A-specific ligand, a KIF20A-specific aptamer, and combinations thereof.

In other embodiments, the first probe for detecting SLC7A5 is a SLC7A5 RNA-specific nucleic acid binding agent. In certain embodiments, the second probe is selected from the group consisting of an TTK-specific nucleic acid RNA-binding agent, a TRIM59 RNA-specific nucleic acid binding agent, a UHRF1 RNA-specific nucleic acid binding agent, a KIF20A RNA-specific nucleic acid binding agent, and combinations thereof. In some embodiments, the kit further comprising a solid support to which the first probe and/or the second probe(s) is/are immobilized or can be immobilized. In certain embodiments, the first probe and/or the second probe is selected from the group consisting of RNA, cDNA, cRNA, and RNA-DNA hybrids. In particular embodiments the SLC7A5 probe is complementary to at least a 20 nucleotides of a nucleic acid sequence consisting of SEQ ID NO: 6. In some embodiments, the second probe is a nucleic acid probe complementary to at least a 20 nucleotide sequence of a nucleic acid sequence selected from the group consisting of SEQ ID NOS: 7, 8, 9, and 10. the first probe and/or the second probe further comprises a detectable label in some embodiments.

In another aspect, the disclosure provides methods of treating a neoplasm in a patient, comprising administering a therapeutically effective amount of an SLC7A5-specific antibody, or SLC7A5-specific binding fragment thereof, to a patient in need thereof, and detecting a decrease in the presence of the neoplasm, or symptoms resulting from the neoplasm.

In yet another aspect, the disclosure provides a cell surface SLC7A5-targeted agent for treating a neoplastic cell growth. The cell surface SLC7A5-targeted agent comprises a SLC7A5 binding component and a therapeutic component. The SLC7A5 binding component targets the therapeutic component to the neoplastic cell growth, and thereby treats the cancer. The SLC7A5 binding component and the therapeutic component, therefore, act together to treat the neoplasm.

In certain embodiments, the SLC7A5 binding component is a SLC7A5-specific antibody or SLC7A5-specific binding portions thereof. In other embodiments, the SLC7A5 binding component is a SLC7A5 ligand. In particular embodiments, the SLC7A5 binding component is a natural ligand, synthetic small molecule, chemical, nucleic acid, peptide or protein.

In some embodiments, the therapeutic component is a chemotherapeutic agent such as Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, Vinorelbine, or combinations thereof. In a particular embodiment, the therapeutic component is in a liposome formulation.

In other embodiments, the therapeutic component is a radioisotope such as ⁹⁰Y, ¹²⁵I, ¹³¹I, ²¹¹At or ²¹³Bi.

In still other embodiments, the therapeutic component is a toxin which kills or induces the killing of the targeted neoplastic cell. Such toxins for use in the disclosure include Pseudomonas exotoxin, diphtheria toxin, plant ricin toxin, plant abrin toxin, plant saporin toxin, plant gelonin toxin and pokeweed antiviral protein.

In particular embodiments, the SLC7A5 binding component of the cell surface SLC7A5-targeted therapeutic agent binds to the surface of the target cell, and the therapeutic element is internalized and arrests growth of the cell, compromises viability of the cell, or kills the cell.

In another aspect, the disclosure provides a method of treating a neoplasm in a subject by administering any of the cell surface SLC7A5-targeted therapeutic agents described above. In certain embodiments, the neoplasm is a breast cancer, an ovarian cancer, a myeloma, a lymphoma, a melanoma, a sarcoma, a leukemia, a retinoblastoma, a hepatoma, a glioma, a mesothelioma, or a carcinoma. In further embodiments, the neoplasm is from a tissue such as blood, bone marrow, spleen, lymph node, liver, thymus, kidney, brain, skin, gastrointestinal tract, eye, breast, prostate, or ovary. In particular embodiments, the subject is a human patient, such as a human patient suffering from a disease or disorder caused by the presence of the neoplasm.

In yet another aspect, the disclosure provides vaccines for treating or preventing a neoplasm. These vaccines of the disclosure include a SLC7A5 polypeptide, or SLC7A5 polypeptide subsequence thereof, and at least one pharmaceutically acceptable vaccine component. In certain embodiments, the SLC7A5 polypeptide or polypeptide subsequence is a human SLC7A5 polypeptide sequence having an amino acid sequence of SEQ ID NO:1. In particular embodiments, the SLC7A5 polypeptide subsequence is at least eight amino acids long, and in certain embodiments, functions as a hapten.

In certain embodiments, the vaccine formulation comprises an adjuvant or other pharmaceutically acceptable vaccine component. In particular embodiments, the adjuvant is aluminum hydroxide, aluminum phosphate, calcium phosphate, oil emulsion, a bacterial product, whole inactivated bacteria, an endotoxins, cholesterol, a fatty acid, an aliphatic amine, a paraffinic compound, a vegetable oil, monophosphoryl lipid A, a saponin, or squalene.

In another aspect, the disclosure provides a method of treating or preventing a neoplasm in a subject by administering any of the SLC7A5 vaccines described above. In certain embodiments, the neoplasm to be treated is a breast cancer, an ovarian cancer, a myeloma, a lymphoma, a melanoma, a sarcoma, a leukemia, a retinoblastoma, a hepatoma, a glioma, a mesothelioma, or a carcinoma. In further embodiments, the neoplasm is from a tissue such as blood, bone marrow, spleen, lymph node, liver, thymus, kidney, brain, skin, gastrointestinal tract, eye, breast, prostate, or ovary. In particular embodiments, the subject is a human patient.

### BRIEF DESCRIPTION OF THE FIGURES

The foregoing and other objects of the present disclosure, the various features thereof, as well as the disclosure itself may be more fully understood from the following description, when read together with the following accompanying drawings:

FIG. 1 is a graphic representation of the differential expression of SLC7A5 RNA in NSCLC tumors relative to normal lung samples from patients as measured by qRT-PCR, where results are expressed as normalized ratio of SLC7A5 between patients' samples and H23 tumor lung cell line calibrator. The results shown in this figure are based on sample size of NSCLC patients n=11 and Normal patient n=15. Unpaired Student's t test was done and equal to p=0.0088.

FIG. 2 is a graphic representation of the differential expression of SLC7A5 in breast, ovarian, colorectal and prostate cancers, relative to normal samples in these tissues as measured by qRT-PCR, where results are expressed as normalized ratio of SLC7A5 between patients' samples from breast, ovarian, colorectal and prostate samples and H23 tumor lung cell line calibrator.

FIG. 3 is a graphic representation of ROC curves for SLC7A5 in lung cancer, where the large dashed lines represent 95% confidence limits and are based on a group of normal lung and NSCLC samples (n=15N+11T).

FIGS. 4A and 4B are graphic representations of ROC curves of secreted SLC7A5 and TRIM59 in combination at high accuracy (A) and at 100% specificity (B) in NSCLC.

FIG. 5 is a graphic representation of the differential expression of SLC7A5 RNA in breast cancer, where results are expressed as normalized ratio of SLC7A5 between patients' samples and H23 tumor lung cell line calibrator. 13.4-fold increase in the expression of SLC7A5 RNA was observed in breast cancer samples relative to normal samples. Breast cancer patients n=17; Normal patient n=10. Unpaired Student's t test was done and p<0.0086.

FIG. 6 is a graphic representation of the differential expression of SLC7A5 RNA in different stage breast cancer tumors, where results are expressed as normalized ratio of SLC7A5 RNA expression between patient samples and H23 tumor lung cell line calibrator. Breast cancer patients at stage 1 (n=7) and stage 2 (n=10) were compared to normal breast samples (n=10). Non-parametric Kruskal-Wallis test (p=0.0001) with Dunn's multiple comparison test was run to assess the significance of SLC7A5 expression between normal and stage I breast cancer patients (p<0.01); normal and stage II breast cancer patients (p<0.001) and between stage I and stage II breast cancer patients.

FIG. 7 is a graphic representation of ROC curves for SLC7A5 in breast cancer, where the large dashed lines represent 95% confidence limits and are based on a group of normal and breast cancer samples (N=10N+17T).

FIG. 8 is a graphic representation of ROC curves of secreted biomarkers (SLC7A5 and TRIM59) separately and in combination at high accuracy and at 100% specificity in breast cancer (the same cut-off values for both setting), where the large dashed lines represent 95% confidence limits and are based on a group of normal and breast cancer samples (N=10N+17T). FIG. 8 are graphic representations of ROC curves of secreted biomarkers (SLC7A5 and TRIM59) separately and in combination at high accuracy (A) and at 100% specificity (B) in breast cancer, where the large dashed lines represent 95% confidence limits and are based on a group of normal and breast cancer samples (N=10N+17T).

FIG. 9 is a graphic representation of the differential expression of SLC7A5 RNA in ovarian cancer, where the results are expressed as normalized ratio of SLC7A5 between patients' samples and H23 tumor cell line calibrator. A 4.2-fold increase in the expression of SLC7A5 RNA was observed in ovarian cancer samples relative to normal samples. Ovarian cancer patients n=17 (n=8 stage I/II; n=9 stage III); Normal patient n=10. Unpaired Student's t test was done and p<0.0001.

FIG. 10 is a graphic representation of ROC curves for SLC7A5 in ovarian cancer, where the large dashed lines represent 95% confidence limits and are based on a group of normal and ovarian cancer samples (N=10N+17T).

FIGS. 11A and 11B are graphic representations of ROC curves of secreted biomarkers (SLC7A5 and TRIM59) in combination at high accuracy (A) and at 100% specificity (B) in ovarian cancer, where the large dashed lines represent 95% confidence limits and are based on a group of normal and ovarian cancer samples (N=10N+17T).

FIG. 12 is a graphic representation of the differential expression of SLC7A5 RNA in colorectal cancer, where the results are expressed as normalized ratio of SLC7A5 between patients' samples and H23 tumor cell line calibrator.

FIG. 13 is a graphic representation of the ROC curves for SLC7A5 in colorectal cancer, where the large dashed lines represent 95% confidence limits and are based on a group of normal and colorectal cancer samples (n=10N+10T matched).

FIGS. 14A and 14B are graphic representations of the ROC curves of secreted biomarkers (SLC7A5 and TRIM59) in combination at high accuracy (A) and at 100% specificity (B) in colorectal cancer, where the large dashed lines represent 95% confidence limits and are based on a group of normal and colorectal cancer samples (n=10N+10T matched). HA: high accuracy. Spec: specificity.

FIG. 15 is a representation of representative nucleotide sequences for KIF20A, UHRF1, TTK, TRIM59, and SLC7A5.

FIG. 16 is a representation of representative amino acid sequences for KIF20A, UHRF1, TTK, TRIM59, and SLC7A5.

### DETAILED DESCRIPTION OF THE INVENTION

Patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

### 1.1 General

The present disclosure provides, in part, methods and kits for diagnosing, detecting, or screening a test sample, such as a fluid or cell sample, for tumorigenic potential and neoplastic characteristics such as aberrant growth. The disclosure also provides methods and reagents for preventing and treating a neoplasm in a patient. The disclosure further allows for the improved clinical treatment and management of tumors by providing a method that detects the expression level of a gene or genes identified as markers for cancer.

One such gene expresses the biomarker SLC7A5. SLC7A5 has an amino acid permease activity is implicated in amino acid metabolism. The SLC7A5 protein is highly expressed several normal tissues and organs (e.g., in adult lung, liver, brain, thymus, retina and some other tissues).

Typically, a gene will affect the phenotype of the cell through its expression at the protein level. Mutations in the coding sequence of the gene can alter its protein product in such a way that the protein does not perform its intended function appropriately. Some mutations, however, affect the levels of protein expressed in the cell without altering the functionality of the protein, itself. Such mutations directly affect the phenotype of a cell by changing the delicate balance of protein expression in a cell. Therefore, an alteration in a gene's overall activity can be measured by determining the level of expression of the protein product of the gene in a cell.

Accordingly, one aspect of the disclosure provides a method for identifying a cancerous cell. The method utilizes protein-targeting agents to identify protein markers, such as SLC7A5, in a potentially cancerous cell sample or potentially cancerous fluid sample. Increased levels of expression of particular protein markers in a cell or fluid sample and a decreased expression level of other protein markers in a cell or fluid sample indicate the presence of a neoplasm.

As used herein, the term “cancer” refers to a disease condition in which a tissue or cells exhibit aberrant, uncontrolled growth and/or lack of contact inhibition. A cancer can be a single cell or a tumor composed of hyperplastic cells. In addition, cancers can be malignant and metastatic, spreading from an original tumor site to other tissues in the body. In contrast, some cancers are localized to a single tissue of the body.

As used herein, a “cancer cell” or “neoplastic cell” is a cell that shows aberrant cell growth, such as increased, uncontrolled cell proliferation and/or lack of contact inhibition. A cancer cell can be a hyperplastic cell, a cell from a cell line that shows a lack of contact inhibition when grown in vitro, or a cancer cell that is capable of metastasis in vivo. In addition, cancer cells include cells isolated from a tumor or tumors.

As used herein, a “tumor” is a collection of cells that exhibit the characteristics of cancer cells. Non-limiting examples of cancer cells include melanoma, ovarian cancer, ovarian cancer, renal cancer, osteosarcoma, lung cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma. Cancer cells are also located in the blood at other sites, and include, but are not limited to, lymphoma cells, melanoma cells, sarcoma cells, leukemia cells, retinoblastoma cells, hepatoma cells, renal cancer cells, osteosarcoma cells, myeloma cells, glioma cells, mesothelioma cells, and carcinoma cells.

Cancer cells may also have the ability to metastasize to other tissues in the body. “Metastasis” is the process by which a cancer cell is no longer confined to the tumor mass, and enters the blood stream, where it is transported to a second site. Upon entering the other tissue, the cancer cell gives rise to a second situs for the disease and can take on different characteristics from the original tumor. Nevertheless, the new tumor retains characteristics from the tissue from which it derives, allowing for clinical identification of the type of cancer no matter where in the body a cancer cell or group of cells metastasizes. The process of metastasis has been studied extensively and is known in the art (see, e.g., Hendrix et al. (2000) Breast Cancer Res. 2(6): 417-22). Metastasized cells may be isolated from tissues including, but not limited to, blood, bone marrow, lymph node, liver, thymus, kidney, brain, skin, gastrointestinal tract, breast, and prostate.

As used herein, the term “tumorigenic potential” means ability to give rise to either benign or malignant tumors. Tumorigenic potential may occur through genetic mechanisms such as mutation or through infection with vectors such as viruses and bacteria.

The present diagnostic method can also be used to detect a disorder resulting from the presence of a damaged cell. As used in accordance with the disclosure, a “damaged cell” is a cell that is non-neoplastic, but that has been otherwise injured. For example, the non-neoplastic damaged cell may be a cell infected with a pathogen, such as a virus, a bacterium, or a parasite. In one non-limiting example, the cells may be damaged by infection with a multi-cellular parasite, or damaged by the effects of infection by a parasite. Such non-limiting parasites include schistosomes, plasmodiums, trypanosomes, Leishmania, and Taxoplasma.

The term “protein markers” as used herein means any protein, peptide, polypeptides, group of peptides, polypeptides or proteins expressed from a gene, whether chromosomal, extrachromosomal, endogenous, or exogenous, which may produce a cancerous or non-cancerous phenotype in the cell or the organism.

Protein markers can have any structure or conformation, and can be in any location within a cell, including on the cell surface. Protein markers can also be secreted from the cell into an extracellular matrix or directly into the blood or other biological fluid. Protein markers can be a single polypeptide chain or peptide fragments of a polypeptide. Moreover, they can also be combinations of nucleic acids and polypeptides as in the case of a ribosome. Protein markers can have any secondary structure combination, any tertiary structure, and come in quaternary structures as well.

One useful protein marker used to identify a neoplastic disease is SLC7A5 protein. Examples of SLC7A5 amino acid sequences include, but are not limited to, GenBank Accession Nos.Q01650, NP_{—}003477, ABM84264, EAW95375, AAH42600, AAH39692, EAW95373, EAW95374, NP_{—}001041629, NP_{—}055085, NP_{—}620172, NP_{—}004164, NP_{—}055146, BAD97330, BAD96867, and AAX42512.

Other useful protein markers include, but are not limited to, TRIM59, TTK, KIF20A, and UHRF1.

As used herein, “gene” means any deoxyribonucleic acid sequence capable of being translated into a protein or peptide sequence. The gene is a DNA sequence that may be transcribed into an mRNA and then translated into a peptide or protein sequence. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a gene product.

As used herein, the term “protein-targeting agent” or “protein binding agent” means a molecule capable of binding, interacting, or associating with a protein or a portion of a protein. Such binding or interactions can include ionic bonds, van der Waals interactions, London forces, covalent bonds, and hydrogen bonds. The target protein can be bound in a receptor-binding pocket, on its surface, or any other portion of the protein that is accessible to binding or interactions with a molecule. Protein-targeting agents include, but are not limited to, proteins, peptides, ligands, peptidomimetic compounds, inhibitors, organic molecules, aptamers, or combinations thereof.

As used herein, the term “inhibitor” means a compound that prevents a biomolecule, e.g., a protein, nucleic acid, or ribozyme, from completing or initiating a reaction. An inhibitor can inhibit a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme. In some embodiments, the inhibitor can be nucleic acid molecules including, but not limited to, siRNA that reduce the amount of functional protein in a cell.

As used herein, the term “greater than” means more than, such as when the level of expression for a particular marker in test sample is detectably more than the level of expression for the same marker in a control sample. In these circumstances, expression analyses are qualitatively determined. The level of expression for a marker can also be determined quantitatively in test and control samples. In quantitative studies, the level of expression for a marker in a test sample is greater than the level of expression for the same marker in a control sample when the level of expression in the test sample is quantifiably determined to be at least about 10% more than the level of expression in the control sample.

As used herein, “about” means a numeric value having a range of ±10% around the cited value. For example, a range of “about 1.5 times to about 2 times” includes the range “1.35 times to 2.2 times” as well as the range “1.65 times to 1.8 times,” and all ranges in between.

For purposes of the disclosure, the term “capture probe” is intended to mean any agent capable of binding a gene product in a complex cell sample or fluid sample. Capture probes can be disposed on the derivatized solid support utilizing methods practiced by those of ordinary skill in the art through a process called “printing” (see, e.g., Schena et. al., (1995) Science, 270(5235): 467-470). The term “printing”, as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce a focused microarray for this aspect. The capture probes may be antibodies, fragments thereof, or any other molecules capable of binding a protein (herein termed “protein capture probes”). These probes may be attached to a solid support at predetermined positions.

### 1.2 Samples to be Tested

In the present disclosure, samples containing tumor cell markers including SLC7A5 and potentially other protein markers are taken and screened relative to control samples. Samples can be fluid or cell samples.

As used herein, the term “fluid sample” refers to a liquid sample. Such samples can be isolated from biological fluids, e.g., urine, blood, lymph, pleural fluid, pus, marrow, cartilaginous fluid, saliva, seminal fluid, amniotic fluid, menstrual blood, lacrimal secretions, vaginal secretions, sweat, and spinal fluid. Such samples can control protein markers secreted from cells. Fluid samples can also be isolated from tissues isolated from a subject. For instance, the tissues can be isolated from organs including, but not limited to, brain, kidney, cartilage, lung, ovary, lymph nodes, salivary glands, breast, prostate, testes, uterus, skin and bone. A tissue sample can also be obtained from necrotic material isolated from a tumor or tumors. Such cell or group of cells may show aberrant cell growth, such as increased, uncontrolled cell proliferation and/or lack of contact inhibition. A “test fluid sample” is a fluid sample that is obtained or isolated from a subject potentially suffering from a neoplastic disease. Fluid samples potentially include a neoplastic cell or group of cells or markers from neoplastic cells. Thus, the test fluid sample can include, for example, a cancer cell that can be a hyperplastic cell, a cell from a cell line that shows a lack of contact inhibition when grown in vitro, or a cancer cell that is capable of metastasis in vivo, or a protein marker secreted or originating from a cancer cell.

As used herein, the term “test cell sample” refers to a cell, group of cells, or cells isolated from potentially cancerous tumor tissues. A test cell sample is one that potentially exhibits tumorigenic potential, metastatic potential, or aberrant growth in vivo or in vitro. A test cell sample can be isolated from any tissue, including, but not limited to, blood, bone marrow, muscle, spleen, lymph node, liver, lung, colon, thymus, kidney, brain, skin, gastrointestinal tract, eye, breast, and prostate. A test cell sample includes the cytoplasmic fraction of a cell in the cell sample.

As used herein, the term “non-neoplastic control cell sample” refers to a cell or group of cells that is exhibiting noncancerous normal characteristics for the particular cell type from which the cell or group of cells was isolated. The control cell has the same lineage as the test cell to which it is compared. A control cell sample does not exhibit tumorigenic potential, metastatic potential, or aberrant growth in vivo or in vitro. A control cell sample can be isolated from normal tissues in a subject that is not suffering from cancer. It may not be necessary to isolate a control cell sample each time a cell sample is tested for cancer as long as the nucleic acids isolated from the normal control cell sample allow for probing against the focused microarray during the testing procedure. The control cell sample may be the cytoplasmic fraction obtained from control cells.

In certain embodiments, the cell to be tested is a test damaged cell from a tissue, for example, from a damaged tissue (e.g., necrotic tissue), or from a type of cell that is infected by the pathogen. For example, the hepatitis B virus typically infects only liver cells; thus, a damaged cell (i.e., a liver cell infected by hepatitis B virus) is from a tissue (i.e., liver). Similarly, the Human Immunodeficiency Virus (HIV) typically infects only CD4+ T cells and macrophages; thus a damaged cell (e.g., a CD4+ T cell infected with HIV) is from a tissue (i.e., blood or bone marrow).

Note that in some limited situations, infection by a virus may cause such a damaged cell to become neoplastic. For example, some B cells, when infected with the Epstein Barr Virus (EBV), become neoplastic. Such a neoplastic B cell, although damaged by virtue of its infection with a virus, is included herein as a “neoplastic cell”, not a damaged cell.

In certain embodiments, the test neoplastic cell is from a tissue, for example, from a biopsy of a hyperplastic tissue (e.g., a lump in the breast). Non-limiting examples of tissues from which a test neoplastic cell can be from include blood, bone marrow, spleen, lymph node, liver, thymus, kidney, brain, skin, gastro-intestinal tract, eye, breast, prostate, and ovary.

In accordance with the disclosure, the neoplastic cell may be from a patient, such as a human, who is suffering from a disease or disorder where the disease or disorder is caused by the presence of the neoplastic cell. For example, where the neoplastic cell is a neoplastic melanoma cell, the disease is a cancer of the melanoma cell (i.e., the cancer is melanoma which is caused by aberrant cell growth and metastasis of the neoplastic cell).

In accordance with the disclosure, the damaged cell may be from a patient, such as a human, who is suffering from a disease or disorder where the disease or disorder is caused by the presence of the damaged cell. For example, where the damaged cell is infected with a pathogen, the disease is an infection caused by the presence of those damaged cells infected by the pathogen or lack thereof (e.g., AIDS caused by the lack of CD4⁺ T cells which were infected by the HIV virus).

As used herein, a “patient suffering from a disease or disorder” means a patient who has the clinical manifestations and/or symptoms of a disease or disorder. In certain situations, a patient with a disease or disorder may be asymptomatic, and yet still have clinical manifestations of the disease or disorder. For example, a patient suffering from leukemia, may not be symptomatic (e.g., may not be sick or weak), but shows the clinical manifestation in that the patient has a larger number of white blood cells as compared to a healthy individual of the same age and weight. In another non-limiting example, a patient suffering from infection with a virus (e.g., HIV), may not be symptomatic (e.g., may not show a diminished CD4+ T cell count), but shows the clinical manifestation in that the patient has anti-HIV antibodies.

Aspects of the disclosure provides methods for diagnosing cancer in a test cell sample by detecting SLC7A5 protein using a dipstick assay, Western blots, dot blots, and Enzyme-Linked Immunosorbent Assays (“ELISA's”).

SLC7A5 can also be detected along with different cancer markers using a protein microarray. The methods can be practiced using a microarray composed of capture probes affixed to a derivatized solid support such as, but not limited to, glass, nylon, metal alloy, or silicon. Non-limiting examples of derivatizing substances include aldehydes, gelatin-based substrates, epoxies, poly-lysine, amines and silanes. Techniques for applying these substances to solid surfaces are well known in the art. In useful embodiments, the solid support can be comprised of nylon.

The level of expression of SLC7A5 in the potentially cancerous test cell sample or potentially cancerous test fluid sample is compared to the level of expression of SLC7A5 in a non-neoplastic control cell or control fluid sample of the same tissue type or lineage. If the expression of SLC7A5 in the potentially cancerous cell or fluid sample is greater than the expression of SLC7A5 in the non-neoplastic control cell or fluid sample, then cancer is indicated. In some embodiments, the test cell or fluid sample is tumorigenic if the level of expression of SLC7A5 in the potentially cancerous cell or fluid sample is at least 1.5 times greater, at least 2 times greater, at least 4 times greater, at least 6 times greater, at least 8 times greater, at least 8 times greater, and at least 12 times greater, at least 15 times greater, or at least 20 times greater, than the level of expression of SLC7A5 in the non-neoplastic control cell or non-neoplastic fluid sample.

In embodiments in which test tissue and cell samples are used, cell samples can be isolated from human tumor tissues using means that are known in the art (see, e.g., Vara et al. (2005) Biomaterials 26(18):3987-93; Iyer et al. (1998) J. Biol. Chem. 273(5):2692-7). For example, the cell sample can be isolated from the ovary of a human patient with ovarian cancer. Other cancer cells that can be obtained include, but are not limited to, prostate cancer cells, melanoma cancer cells, osteosarcoma cancer cells, glioma cells, colon cancer cells, lung cancer cells, breast cancer cells, colon cancer and colorectal cancer cells, and leukemia cells. Cancer cells can metastasize to distant locations in the body. Non-limiting sites of metastases can include, but are not limited to, ovarian, bone, blood, lung, skin, brain, adipose tissue, muscle, gastrointestinal tissues, hepatic tissues, and kidney. Alternatively, the cell test or control cell sample can be obtained from a cell line. Cell lines can be obtained commercially from various sources (e.g., American Type Culture Collections, Mannassas, Va.). Alternatively, cell lines can be produced using techniques well known in the art.

In addition, the cell sample can be a cell line. Cancer cell lines can be created by one with skill in the art and are also available from common sources, such as the ATCC cell biology collections (American Type Culture Collections, Mannassas, Va.).

The present disclosure allows for the detection of cancer in tissues that are of mixed cellular populations such as a mixture of cancer cells and normal cells. In such cases, cancer cells can represent as little as 40% of the tissue isolated for the present disclosure to determine that the cell sample is tumorigenic. For example, the cell sample can be composed of 50% cancer cells for the present disclosure to detect tumorigenic potential. Cell samples composed of greater than 50% tumorigenic cells can also be used in the present disclosure. It should be noted that cell samples can be isolated from tissues that are less than 40% tumorigenic cells as long as the cell sample contains a portion of cells that are at least 40% tumorigenic.

Another aspect of the disclosure provides a method of diagnosing cancer in a fluid sample. In this method, expression of SLC7A5 in the fluid sample is measured. Expression levels for SLC7A5 can be determined using any techniques known in the art. Useful ways to determine such expression levels include, but not limited to, Western blot, protein microarrays, dipstick assays, dot blots, and Enzyme-Linked Immunosorbent Assays (“ELISA”) (see, e.g., U.S. Pat. Nos. 6,955,896, 6,087,012, 3,791,932, 3,850,752, and 4,034,074). Such examples are not intended to limit the potential means for determining the expression of a protein marker in a cell sample. Expression levels of markers in or by potentially cancerous cell samples and normal control cell samples can be compared using standard statistical techniques known to those of skill in the art (see, e.g., Ma et al. (2002) Meth. Mol. Biol. 196:139-45).

The fluid sample can be isolated from a human patient by a physician and tested for expression of SLC7A5 using a dipstick or any other method that relies on a solid support, solid state binding, change in color, or electric current. In addition, the cancer cell sample can be isolated from an organism that develops a tumor or cancer cells including, but not limited to, mouse, rat, horse, pig, guinea pig, or chinchilla. Cell samples can be stored for extended periods prior to testing or tested immediately upon isolation of the cell sample from the subject. Cell samples can be isolated by non-limiting methods such as surgical excision, aspiration from soft tissues such as adipose tissue or lymphatic tissue, biopsy, or removed from the blood. These methods are known to those of skill in the art.

In certain embodiments, the level of expression of anti-SLC7A5 antibodies in a fluid sample is detected. The level of expression of anti-SLC7A5 antibodies in a cell sample is detected using ELISA, Western blot, and dot blot. The level of expression of anti-SLC7A5 antibodies can be detected using antibodies or fragments thereof, which are directed against anti-SLC7A5 antibodies. The level of expression of anti-SLC7A5 antibodies can be detected using SLC7A5-specific antibody fragments (e.g., Fab, F(ab)₂, and Fv) or whole antibodies.

A normal or cell sample can be isolated from a human patient by a physician and tested for expression of protein markers using a dipstick or any other method that relies on a solid support, solid state binding, change in color, or electric current. In addition, the cancer cell sample can be isolated from an organism that develops a tumor or cancer cells including, but not limited to, mammals such as mouse, rat, horse, pig, guinea pig, rabbit, or chinchilla. Cell samples can be isolated by non-limiting methods such as surgical excision, aspiration from soft tissues such as adipose tissue or lymphatic tissue, biopsy, or removed from the blood.

These methods are known to those of skill in the art. Cell samples can be stored for extended periods prior to testing or tested immediately upon isolation of the cell sample from the subject.

### 1.3 Nucleic Acid Binding Agents

In another aspect, the method of detecting cancer includes detecting a level of expression of SLC7A5 RNA in a test sample (i.e., neoplastic test cell or test fluid sample) and comparing the level of expression of SLC7A5 RNA detected in the test sample to the level of expression of SLC7A5 RNA detected in the non-neoplastic control sample. If the level of expression of SLC7A5 RNA is greater in the fluid sample than in the non-neoplastic control sample, then cancer is indicated.

As used herein, “nucleic acid binding agent” means a nucleic acid capable of hybridizing with a particular target nucleic acid sequence. Nucleic acid binding agents include any structure that can hybridize with a target nucleic acid such as an mRNA. Nucleic acids can include, but are not limited to, DNA, RNA, RNA-DNA hybrids, siRNA, and aptamers. Moreover, any detectable labels can be used so long as the label does not affect the hybridizing of the nucleic acid with its targeting. Labels include, but are not limited to, fluorophores, chemical dyes, radiolabels, chemiluminescent compounds, colorimetric enzymatic reactions, chemiluminescent enzymatic reactions, magnetic compounds, and paramagnetic compounds.

Examples of SLC7A5 nucleic acid sequences detected in the present disclosure include, but are not limited to, GenBank Accession Nos. NM_{—}003486, AL627071, DQ896766, DQ893338, BC039692, BC042600, DQ893338, CH471114, AC_{—}000148, and NW_{—}001838330.

In certain embodiments, a focused microarray can be used to detect the levels of expression of SLC7A5 with other markers. The term “focused microarray” as used herein refers to a device that includes a solid support with capture probe(s) affixed to the surface of the solid support. In some embodiments, the focused microarray has nucleic acids attached to a solid support. Typically, the support consists of silicon, glass, nylon or metal alloy. Solid supports used for microarray production can be obtained commercially from, for example, Genetix Inc. (Boston, Mass.). Moreover, the support can be derivatized with a compound to improve nucleic acid association. Exemplary compounds that can be used to derivatize the support include aldehydes, poly-lysine, epoxy, silane containing compounds and amines. Derivatized slides can be obtained commercially from Telechem International (Sunnyvale, Calif.).

In the case of nucleic acid binding agents, nucleic acid sequences that are selected for detecting SLC7A5 expression may correspond to regions of low homology between genes, thereby limiting cross-hybridization to other sequences. Typically, this means that the sequences show a base-to-base identity of less than or equal to 30% with other known sequences within the organism being studied. Sequence identity determinations can be performed using the BLAST research program located at the NIH website (world wide web at ncbi.nlm.nih.gov/BLAST). Alternatively, the Needleman-Wunsch global alignment algorithm can be used to determine base homology between sequences (see Cheung et al. (2004) FEMS Immunol. Med. Micorbiol. 40(1):1-9.). In addition, the Smith-Waterman local alignment can be used to determine a 30% or less homology between sequences (see Goddard et al. (2003) J. Vector Ecol. 28:184-9).

Expression levels for the SLC7A5 can be determined using techniques known in the art, such as, but not limited to, immunoblotting, quantitative RT-PCR, microarrays, RNA blotting, and two-dimensional gel-electrophoresis (see, e.g., Rehman et al. (2004) Hum. Pathol. 35(11):1385-91; Yang et al. (2004) Mol. Biol. Rep. 31(4):241-8). Such examples are not intended to limit the potential means for determining the expression of a gene marker in a breast cancer fluid sample.

Other useful nucleic acid binding agents are specific for TTK RNA, KIF20A RNA, TRIM59 RNA and UHRF1 RNA. These agents can be used in combination with binding agents for SLC7A5 to detect neoplastic disease. In particular embodiments, a plurality of RNA for TTK, KIF20A, TRIM59 and UHRF1 are detected with SLC7A5 RNA in a neoplastic test fluid or cell sample. In such embodiments, the level of expression of at least one of TTK, KIF20A, TRIM59 and UHRF1 is 1.5 times greater, at least 2 times greater, at least 5 times greater, or at least 10 or more times greater in a test fluid or cell sample than the level of expression of the same markers in a control fluid or cell sample. The nucleic acid sequences of TTK, KIF20A, TRIM59 and UHRF1 have SEQ ID NOS: 2, 5, 4, and 1, respectively.

### 1.4 Protein-Targeting Agents

Protein marker expression is used to identify tumorigenic potential. Protein markers, such as SLC7A5, can be obtained by isolation from a cell sample, or a fluid sample, using any techniques available to one of ordinary skill in the art (see, e.g., Ausubel et. al., Current Protocols in Molecular Biology, Wiley and Sons, New York, N.Y., 1999). Isolation of protein markers, including SLC7A5, from the potentially tumorigenic cell sample, or from a fluid sample, obtained from a patient potentially suffering or suffering from neoplastic disease, allows for the generation of target molecules, providing a means for determining the expression level of the protein markers in the potentially tumorigenic cell or fluid sample as described below. The protein markers, such as SLC7A5, can be isolated from a tissue or fluid sample isolated from a human subject. SLC7A5 and other protein markers can be isolated from a cytoplasmic fraction or a membrane fraction of the sample. Protein isolation techniques known in the art include, but are not limited to, column chromatography, spin column chromatography, and protein precipitation. SLC7A5 can be isolated using methods that are taught in, for example, Ausubel et al. Current Protocols in Molecular Biology, Vol. 1, John Wiley & Sons, Inc., (1993).

The disclosure provides protein-targeting agents such as binding agents, e.g., SLC7A5-specific antibodies or SLC7A5 binding fragments thereof. These embodiments are described in detail below. Other potential protein targeting agents include, but are not limited to, aptamers, and ligands specific for SLC7A5 peptidomimetic compounds, peptides directed to the active sites of an enzyme, and nucleic acids.

Inhibitors can also be used as protein targeting agents to bind to protein markers. Useful inhibitors are compounds that bind to a target protein, and normally reduce the “effective activity” of the target protein in the cell or cell sample Inhibitors include, but are not limited to, antibodies, antibody fragments, peptides, peptidomimetic compounds, and small molecules (see, e.g., Lopez-Alemany et al. (2003) Am. J. Hematol. 72(4): 234-42; Miles et al. (1991) Biochem. 30(6): 1682-91). Inhibitors can perform their functions through a variety of means including, but not limited to, non-competitive, uncompetitive, and competitive mechanisms.

Protein-targeting agents, including antibodies (described below) can also be conjugated to non-limiting materials such as magnetic compounds, paramagnetic compounds, proteins, nucleic acids, antibody fragments, or combinations thereof. Furthermore, protein-targeting agents can be disposed on an NPV membrane and placed into a dipstick. Protein-targeting agents can also be immobilized on a solid support at pre-determined positions such as in the case of a microarray. For instance, antibodies can be printed or cross-linked via their Fc regions to pre-derivatized surfaces of solid supports. In addition, antibodies can be cross-linked using bifunctional crosslinkers to a functionalized solid support. Such bifunctional crosslinking is well known in the art (see, e.g., U.S. Pat. Nos. 7,179,447 and 7,183,373).

Crosslinking of protein-targeting agents, such as antibodies and other proteins, to a water-insoluble support matrix can be performed with bifunctional agents well known in the art including 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Bifunctional agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates can be employed for protein immobilization.

Protein-targeting agents can be detectably labeled. As used herein, “detectably labeled” means that a targeting agent is operably linked to a moiety that is detectable. By “operably linked” is meant that the moiety is attached to the protein-targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al. The Chemistry and Application of Amino Crosslinking Agents or Aminoplasts, John Wiley & Sons Inc., New York City, N.Y., 1999).

According to the disclosure, a “detectable label” is a moiety that can be sensed. Such labels can be, without limitation, fluorophores (e.g., fluorescein (FITC), phycoerythrin, rhodamine), chemical dyes, or compounds that are radioactive, chemiluminescent, magnetic, paramagnetic, promagnetic, or enzymes that yield a product that may be colored, chemiluminescent, or magnetic. The signal is detectable by any suitable means, including spectroscopic, photochemical, biochemical, immunochemical, electrical, optical or chemical means. In certain cases, the signal is detectable by two or more means. In certain embodiments, protein targeting agents include fluorescent dyes, radiolabels, and chemiluminescent labels, which are examples that are not intended to limit the scope of the disclosure (see, e.g., Gruber et al. (2000) Bioconjug. Chem. 11(5): 696-704).

For example, protein-targeting agents may be conjugated to Cy5/Cy3 fluorescent dyes. These dyes are frequently used in the art (see, e.g., Gruber et al. (2000) Bioconjug. Chem. 11(5): 696-704). The fluorescent labels can be selected from a variety of structural classes, including the non-limiting examples such as 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

Aspects of the present disclosure utilize antibodies, both monoclonal and polyclonal antibodies, as protein targeting agents directed specifically against certain cancer marker proteins, particularly SLC7A5. Anti-SLC7A5 protein antibodies, both monoclonal and polyclonal, for use in the disclosure are available from several commercial sources. Other useful markers to which protein targeting agents such as antibodies can be provided include, but are not limited to, TTK, KIF20A, TRIM59 and UHRF1. SLC7A5, TTK, KIF20A, TRIM59 and UHRF1 antibodies can be administered to a patient orally, subcutaneously, intramuscularly, intravenously, or interperitoneally for in vivo detection and/or imaging. In certain embodiments, SLC7A5 is used alone as a protein marker to diagnose cancer.

Aspects of the disclosure also utilize polyclonal antibodies for the detection of SLC7A5, TTK, KIF20A, TRIM59 and UHRF1. They can be prepared by known methods or commercially obtained.

The term “antibody” is used in the broadest sense and specifically covers single anti-SLC7A5 monoclonal and polyclonal antibodies, as well as anti-SLC7A5 antibody fragments (e.g., Fab, F(ab)₂, and Fv) and anti-SLC7A5 antibody compositions with polyepitopic specificity (including binding and non-binding antibodies).

The term “monoclonal antibody” as used herein refers to an antibody obtained from a population of substantially homogeneous antibodies, i.e., the individual antibodies comprising the population are identical except for possible naturally occurring mutations that may be present in minor-amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. Novel monoclonal antibodies or fragments thereof mean in principle all immunoglobulin classes such as IgM, IgG, IgD, IgE, IgA or their subclasses such as the IgG subclasses or mixtures thereof. IgG and its subclasses are included, such as IgG1, IgG2, IgG2a, IgG2b, IgG3 or IgGM. The IgG subtypes IgG1/kappa and IgG 2b/kapp are also included as embodiments.

As used herein, the term “polyclonal antibodies” means a population of antibodies that can bind to multiple epitopes on an antigenic molecule. A polyclonal antibody is specific to a particular epitope on an antigen, while the entire group of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al. Current Protocols in Molecular Biology, Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present disclosure. Antibodies can be obtained commercially from, e.g., BioMol International LP (Plymouth Meeting, Pa.), BD Biosciences Pharmingen (San Diego, Calif.), and Cell Sciences, Inc. (Canton, Mass.).

The monoclonal antibodies herein include hybrid and recombinant antibodies produced by splicing a variable (including hypervariable) domain of an anti-biomarker protein antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present disclosure can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al. (1990) Nature 348:552-554).

Alternative methods for producing antibodies can be used to obtain high affinity antibodies. Antibodies can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al. (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. Eng. 2: 339-76; U.S. Pat. Nos. 4,833,077, 5,811,524, 5,958,765, 6,413,771, and 6,537,809).

The antibody genes for the genetic manipulations can be isolated, for example from hybridoma cells, in a manner known to the skilled worker. For this purpose, antibody-producing cells are cultured and, when the optical density of the cells is sufficient, the mRNA is isolated from the cells in a known manner by lysing the cells with guanidinium thiocyanate, acidifying with sodium acetate, extracting with phenol, chloroform/isoamyl alcohol, precipitating with isopropanol and washing with ethanol. cDNA is then synthesized from the mRNA using reverse transcriptase. The synthesized cDNA can be inserted, directly or after genetic manipulation, for example by site-directed mutagenesis, introduction of insertions, inversions, deletions or base exchanges, into suitable animal, fungal, bacterial or viral vectors and be expressed in appropriate host organisms. Preference is given to bacterial or yeast vectors such as pBR322, pUC18/19, pACYC184, lambda or yeast mu vectors for the cloning of the genes and expression in bacteria such as E. coli or in yeasts such as Saccharomyces cerevisiae.

The disclosure furthermore relates to cells that synthesize SLC7A5 antibodies. These include animal, fungal, bacterial cells or yeast cells after transformation as mentioned above. They are advantageously hybridoma cells or trioma cells, or hybridoma cells. These hybridoma cells can be produced, for example, in a known manner from animals immunized with SLC7A5 and isolation of their antibody-producing B cells, selecting these cells for SLC7A5-binding antibodies and subsequently fusing these cells to, for example, human or animal, for example, mouse mylemoa cells, human lymphoblastoid cells or heterohybridoma cells (see, e.g., Koehler et al., (1975) Nature 256: 496) or by infecting these cells with appropriate viruses to produce immortalized cell lines. Hybridoma cell lines produced by fusion are particularly useful, mouse hybridoma cell lines are very useful. The hybridoma cell lines of the disclosure secrete antibodies of the IgG type. The binding of the mAb antibodies of the disclosure bind with high affinity to SLC7A5.

The disclosure further includes derivates of these anti-SLC7A5 antibodies, which retain their SLC7A5-binding activity while altering one or more other properties related to their use as a pharmaceutical agent, e.g., serum stability or efficiency of production. Examples of such anti SLC7A5 antibody derivatives include peptides, peptidomimetics derived from the antigen-binding regions of the antibodies, and antibodies, fragments or peptides bound to solid or liquid carriers such as polyethylene glycol, glass, synthetic polymers such as polyacrylamide, polystyrene, polypropylene, polyethylene or natural polymers such as cellulose, Sepharose or agarose, or conjugates with enzymes, toxins or radioactive or nonradioactive markers such as ³H, ¹²³I, ¹²⁵I, ¹³¹I, ³²P, ³⁵S, ¹⁴C, ⁵¹Cr, ³⁶Cl, ⁵⁷Co, ⁵⁵Fe, ⁵⁹Fe, ⁹⁰Y, ⁹⁹ mTc (metastable isomer of Technetium 99), ⁷⁵Se, or antibodies, fragments or peptides covalently bonded to fluorescent/chemiluminescent labels such as rhodamine, fluorescein, isothiocyanate, phycoerythrin, phycocyanin, fluorescamine, metal chelates, avidin, streptavidin or biotin.

The novel antibodies, antibody fragments, mixtures and derivatives thereof can be used directly, after drying, for example freeze drying, after attachment to the above-mentioned carriers or after formulation with other pharmaceutical active and ancillary substances for producing pharmaceutical preparations. Examples of active and ancillary substances which may be mentioned are other antibodies, antimicrobial active substances with a microbiocidal or microbiostatic action such as antibiotics in general or sulfonamides, antitumor agents, water, buffers, salines, alcohols, fats, waxes, inert vehicles or other substances customary for parenteral products, such as amino acids, thickeners or sugars. These pharmaceutical preparations are used to control diseases, to control arthritic disturbances, advantageously disturbances of joint cartilage.

In addition, aptamers can be protein targeting agents. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target of the present disclosure is SLC7A5, and hence the term “SLC7A5 aptamer” or “SLC7A5 nucleic acid ligand” is used. Aptamers may also be made to other biomarkers as well, such as, but not limited to, TTK, KIF20A, TRIM59, and UHRF1. The aptamer can bind to the target by reacting with the target, by covalently attaching to the target, or by facilitating the reaction between the target and another molecule. Aptamers may be comprised of multiple ribonucleotide units, deoxyribonucleotide units, or a mixture of both types of nucleotide residues. Aptamers may further comprise one or more modified bases, sugars or phosphate backbone units as described above.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al. (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al. Nucleic Acids Res., 12:4539-4557 (1984)) on any commercially available DNA/RNA synthesizer. Purification can be performed either by denaturing polyacrylamide gel electrophoresis or by a combination of ion-exchange HPLC (Sproat et al. (1995) Nucleosides and Nucleotides, 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al. (1993) Nucleic Acids Res., 21:3191-3196) and by nucleoside base composition analysis.

There are several techniques that can be adapted for refinement or strengthening of the nucleic acid ligands binding to a particular target molecule or the selection of additional aptamers. One technique has been termed Selective Evolution of Ligands by Exponential Enrichment (SELEX). Compositions and methods for generating aptamer antagonists of the disclosure by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

The detection of cancer markers can also be accomplished using protein microarrays. Protein microarrays can also be used for diagnosis and prediction. Protein microarrays can be prepared by methods disclosed in, e.g., U.S. Pat. Nos. 6,087,102, 6,139,831, and 6,087,103. In addition, protein-targeting agents conjugated to the surface of the protein microarray can be bound by detectably labeled protein markers, including SLC7A5, isolated from a cell sample or a fluid sample. This method of detection can be termed “direct labeling” because the protein marker, which is the target, is labeled. In other embodiments, protein markers can be bound by protein-targeting agents, and then subsequently bound by a detectably labeled antibody specific for the protein marker. These methods are termed “indirect labeling” because the detectable label is associated with a secondary antibody or other protein-targeting agent. An overview of protein microarray technology in general can be found in Mitchell, Nature Biotech. (2002), 20:225-229, the contents of which are incorporated herein by reference.

In addition to detectable moieties, other non-limiting moieties that may be operably linked to a binding agent of the disclosure include, without limitation, a toxin (e.g., a radioactive isotope), an enzyme, an antibody (or a portion thereof), a cytotoxic drug, or a conjugate of these. Where a toxin is operably linked to a binding agent of the disclosure, non-limiting examples of a toxin which can be operably linked to a binding agent of the disclosure include a radioactive isotope, Diptheria toxin, a nuclease (e.g., DNAse or RNAse), a protease, a degradative enzyme, Pseudomonas exotoxin (PE), ricin A or B chains, and ribonuclease A (Fizgerald D., Semin. Cancer Biol. 7: 87-95, 1996).

In some embodiments, the binding agent is an immunotoxin (e.g., an antibody-toxin conjugate or antibody-drug conjugate). Non-limiting examples of immunotoxins include antibody-anthracycline conjugates (Braslawsky G. R. et al., European Patent No. EP0398305), antibody-cytokine conjugates (Gilles, PCT WO9953958), and monoclonal antibody-PE conjugates (Roffler, et al., Cancer Res. 51: 4001-4007, 1991).

### 1.5 Detection of SLC7A5 and Other Markers in Biological Fluids

An aspect of the present disclosure includes an assay for the detection of SLC7A5 and other cancer markers in biological fluid samples using a protein-targeting agent to bind to the SLC7A5 protein. Protein-targeting agents can bind to SLC7A5 protein that is obtained from tissue or biological fluids.

As used herein, the term “biological fluids” means aqueous or semi-aqueous liquids isolated from an organism in which biological macromolecules may be identified or isolated. Biological fluids may be disposed internally as in the case of blood, serum, amniotic fluid, bile, or cerebrospinal fluid. Biological fluids can be excreted as in the non-limiting cases of urine, saliva, sweat, vaginal secretions, seminal fluids, mucosal secretions, lacrimal secretions, seminal fluid, and sebaceous secretions.

For detection of markers in biological fluids, detection devices can be used that are in the form of a “dipstick.” Such devices are known in the art, and have been applied to detecting SLC7A5 protein in serum and other biological fluids (see, e.g. U.S. Pat. No. 4,390,343). In some instances, a dipstick-type device can be comprised of analytical elements where protein-targeting agents, such as antibodies, inhibitors, organic molecules, peptidomimetic compounds, ligands, organic compounds, or combinations thereof, are incorporated into a gel. The gel can be comprised of non-limiting substances such as agarose, gelatin or PVP (see, e.g., U.S. Pat. No. 4,390,343). The gel can be contained within an analytical region for reaction with a protein marker.

The “dipstick” format (exemplified in U.S. Pat. Nos. 5,275,785, 5,504,013, 5,602,040, 5,622,871 and 5,656,503) typically consists of a strip of porous material having a biological fluid sample-receiving end, a reagent zone and a reaction zone. As used herein, the term “reagent zone” means the area within the dipstick in which the protein-targeting agent and the SLC7A5 protein in the biological sample come into contact. By the term “reaction zone”, is meant the area within the dipstick in which an immobilized binding agent captures the protein-targeting agent/protein marker complex.

In certain embodiments, the biological fluid sample is wicked along the assay device starting at the sample-receiving end and moving into the reagent zone. The protein marker(s) to be detected binds to a protein-targeting agent incorporated into the reagent zone, such as a labeled protein-targeting agent, to form a complex. For example, a labeled antibody can be the protein-targeting agent, which complexes specifically with the protein marker. In other examples, the protein-targeting agent can be a receptor that binds to a protein marker in a receptor:ligand complex. In yet other examples, an inhibitor is used to bind to a protein marker, thereby forming a complex with the protein marker targeted by the particular inhibitor. In some examples, peptidomimetic compounds are used to bind to SLC7A5 protein to mimic the interaction of a protein marker with a normal peptide. In other examples, the protein-targeting agent can be an organic molecule capable of associating with the protein marker. In all cases, the protein-targeting agent has a label. The labeled protein-targeting agent-protein marker complex then migrates into the reaction zone, where the complex is captured by another specific binding partner firmly immobilized in the reaction zone. Retention of the labeled complex within the reaction zone thus results in a visible readout.

A number of different types of other useful assays that measure the presence of a protein market are well known in the art. One such assay is an immunoassay. Immunoassays may be homogeneous, i.e. performed in a single phase, or heterogeneous, where antigen or antibody is linked to an insoluble solid support upon which the assay is performed. Sandwich or competitive assays may be performed. The reaction steps may be performed simultaneously or sequentially. Threshold assays may be performed, where a predetermined amount of analyte is removed from the sample using a capture reagent before the assay is performed, and only analyte levels of above the specified concentration are detected. Assay formats include, but are not limited to, for example, assays performed in test tubes, wells or on immunochromatographic test strips, as well as dipstick, lateral flow or migratory format immunoassays.

A lateral flow test immunoassay device may be used in this aspect of the disclosure. In such devices, a membrane system forms a single fluid flow pathway along the test strip. The membrane system includes components that act as a solid support for immunoreactions. For example, porous or bibulous or absorbent materials can be placed on a strip such that they partially overlap, or a single material can be used, in order to conduct liquid along the strip. The membrane materials can be supported on a backing, such as a plastic backing. The test strip includes a glass fiber pad, a nitrocellulose strip and an absorbent cellulose paper strip supported on a plastic backing.

Antibodies that specifically bind with SLC7A5 or other target protein markers can be immobilized on the solid support. The antibodies can be bound to the test strip by adsorption, ionic binding, van der Waals adsorption, electrostatic binding, or by covalent binding, by using a coupling agent, such as glutaraldehyde. For example, the antibodies can be applied to the conjugate pad and nitrocellulose strip using standard dispensing methods, such as a syringe pump, airbrush, ceramic piston pump or drop-on-demand dispenser. A volumetric ceramic piston pump dispenser can be used to stripe antibodies that bind the analyte of interest, including a labeled antibody conjugate, onto a glass fiber conjugate pad and a nitrocellulose strip.

The test strip can be treated, for example, with sugar to facilitate mobility along the test strip or with water-soluble non-immune animal proteins, such as albumins, including bovine (BSA), other animal proteins, water-soluble polyamino acids, or casein to block non-specific binding sites.

### 1.6 Cancer Diagnosis and Prediction Analysis

Cancer diagnoses can be performed by comparing the levels of expression of a protein marker, such as SLC7A5, or a set of protein markers including SLC7A5, in a potentially neoplastic cell sample to the levels of expression for a protein marker or a set of protein markers in a normal control cell sample of the same tissue type. Alternatively, the level of expression of a protein marker, such as SLC7A5, or a set of protein markers in a potentially cancerous cell sample, is compared to a reference group of protein markers that represents the level of expression for a protein marker or a set of protein markers in a normal control population (herein termed “training set”). The training set also includes the data for a population that has a known tumor or class of tumors. This data represents the average level of expression that has been determined for the neoplastic cells isolated from the tumor or class of tumors. It also has data related to the average level of expression for a protein marker or set of protein markers for normal cells of the same cell type within a population. In these embodiments, the algorithm compares newly generated expression data for a particular protein marker or set of protein markers from a cell sample isolated from a patient containing potentially neoplastic cells to the levels of expression for the same protein marker or set of protein markers in the training set. The algorithm determines whether a cell sample is neoplastic or normal by aligning the level of expression for a protein marker or set of protein markers with the appropriate group in the training set. In certain embodiments, software for performing the statistical manipulations described herein can be provided on a computer connected by data link to a data generating device, such as a microarray reader.

Class prediction algorithms can be utilized to differentiate between the levels of expression of markers in a cell sample and the levels of expression of markers in a normal cell sample (Vapnik, The Nature of Statistical Learning Theory, Springer Publishing, 1995). Exemplary, non-limiting algorithms include, but are not limited to, compound covariate predictor, diagonal linear discriminant analysis, nearest neighbor predictor, nearest centroid predictor, and support vector machine predictor (Simon et al. Design and Analysis of DNA Microarray Investigations: An Artificial Intelligence Milestone., Springer Publishing, 2003). These statistical tests are well known in the art, and can be applied to ELISA or data generated using other protein expression determination techniques such as dot blotting, Western blotting, and protein microarrays (see, e.g., U.S. Publ. No. 2005/0239079).

It should be recognized that statistical analysis of the levels of expression of protein markers in a cell sample to determine cancer state does not require a particular algorithm or set of particular algorithms. Any algorithm can be used in the present disclosure so long as it can discriminate between statistically significant and statistically insignificant differences in the levels of expression of protein markers in a cell sample as compared to the levels of expression of the same protein markers in a normal cell sample of the same tissue type. In this case, a test sample is considered cancerous or malignant if the expression of one or more protein marker is above a cut-off value established for one or all markers in normal or control samples.

In some embodiments, an increased level of expression in the potentially cancerous cell sample, or fluid sample, indicates that cancer cells exist in the cell sample. In such cancerous samples, protein markers showing increased levels of expression include, but are not limited to, SLC7A5, as well as TTK, KIF20A, TRIM59 and UHRF1. The algorithm makes the class prediction based upon the overall levels of expression found in the cell sample as compared to the levels of expression in the training set. It should be noted that, in some instances, SLC7A5 can be used to classify a sample as either neoplastic or normal. Two, three, four, five, six, or more protein markers, including SLC7A5, can also be used to properly classify a cell sample as neoplastic or normal.

The type of analysis detailed above compares the level of expression for the protein marker(s) in the cell sample to a training set containing reference groups of protein that are representative of a normal population and a neoplastic population. In certain embodiments, the training set can be obtained with kits that can be used to determine the level of expression of protein marker(s) in a patient cell sample. Alternatively, an investigator can generate new training sets using protein expression reference groups that can be obtained from commercial sources such as Asterand, Inc. (Detroit, Mich.). Comparisons between the training sets and the cell samples are performed using standard statistical techniques that are well known in the art, and include, but are not limited to, the ArrayStat 1.0 program (Imaging Research, Inc., Brock University, St. Catherine's, Ontario, Calif.). Statistically significant increased levels of expression in the cell sample of protein marker(s) indicate that the cell sample contains a cancer cell or cells with tumorigenic potential. Also, standard statistical techniques such as the Student T test are well known in the art, and can be used to determine statistically significant differences in the levels of expression for protein markers in a patient cell sample (see, e.g., Piedra et al. (1996) Ped. Infect. Dis. J. 15:1). In particular, the Student T test is used to identify statistically significant changes in expression using protein microarray analysis or ELISA analysis (see, e.g., Piedra et al. (1996) Ped. Infect. Dis. J. 15:1).

### 1.7 Kits

Aspects of the disclosure additionally provide kits for detecting neoplasms such as ovarian, lung, breast, colon and prostate cancers in a cell or a fluid sample. The kits include targeting agents for the detection of SLC7A5 or SLC7A5 and at least one of biomarkers TTK, KIF20A, TRIM59 and/or UHRF1. In certain embodiments, kits include targeting agents for the detection of SLC7A5. A patient that potentially has a tumor or the potential to develop a tumor (“in need thereof”) can be tested for the presence of a tumor or tumor potential by determining the level of expression of targeting agents in a cell or fluid sample derived from the patient.

The kit comprises labeled binding agents capable of detecting SLC7A5 or SLC7A5 and at least one of SLC7A5, TTK, KIF20A, TRIM59 and/or UHRF1 in a biological sample, as well as means for determining the amount of these protein markers in the sample, and means for comparing the amount of the protein markers in the potentially cancerous sample with a standard (e.g., normal non-neoplastic control cells). The binding agents can be packaged in a suitable container. The kit can further comprise instructions for using the compounds or agents to detect the protein markers, as well as other neoplasm-associated markers. Such a kit can comprise, e.g., one or more antibodies, or biomarker-specific binding fragments thereof as binding agents, that bind specifically to at least a portion of a protein marker.

In particular, kits comprise labeled binding agents capable of binding to and detecting SLC7A5, as well as means determining the amount of SLC7A5 in the sample, and means for comparing the amount of the protein markers in the potentially cancerous sample with a standard (e.g., normal non-neoplastic control cells). Such a kit can comprise, e.g., one or more antibodies, or biomarker-specific binding fragments thereof as binding agents, that bind specifically to at least a portion of a SLC7A5.

The kit can also contain a probe for detection of housekeeping protein expression. These probes advantageously allow health care professionals to obtain an additional data point to determine whether a specific or general cancer treatment is working so SLC7A5 levels can be used to monitor the success of cancer treatment, and are used to normalize the signal obtained between patients. As used herein, the term “housekeeping proteins” refers to any protein that has relatively stable or steady expression at the protein level during the life of a cell. Housekeeping proteins can be protein markers that show little difference in expression between cancer cells and normal cells in a particular tissue type. Examples of housekeeping proteins are well known in the art, and include, but are not limited to, isocitrate lyase, acyltransferase, creatine kinase, TATA-binding protein, hypoxanthine phosphoribosyl transferase 1, and guanine nucleotide binding protein, beta polypeptide 2-like 1 (see, e.g., Pandey et al. (2004) Bioinformatics 20(17): 2904-2910). In addition, the housekeeping proteins are used to identify the proper signal level by which to compare the cell sample signals between proteins from different or independent experiments. The probes can be any binding agents such as labeled antibodies, or fragments thereof, specific for the housekeeping proteins. Alternatively or additionally, the probes can be inhibitors, peptidomimetic compounds, peptides and/or small molecules.

Data related to the levels of expression of the selected protein marker in normal tissues and neoplasms can be supplied in a kit or individually in the form of a pamphlet, document, floppy disk, or computer CD. The data can represent patient groups developed for a particular population (e.g., Caucasian, Asian, etc.) and is tailored to a particular cancer type. Such data can be distributed to clinicians for testing patients for the presence of a neoplasm. A clinician obtains the levels of expression for a protein marker or set of protein markers in a particular patient. The clinician then compares the expression information obtained from the patient to the levels of expression for the same protein marker or set of protein markers that had been determined previously for both normal control and cancer patient groups. A finding that the level of expression for the protein marker or the set of protein markers is similar to the normal patient group data indicates that the cell sample obtained from the patient is not neoplastic. A finding that the level of expression for the protein marker or the set of protein markers is similar to the cancer patient group data indicates that the cell sample obtained from the patient is neoplastic.

1.8 Testing

The diagnostic methods according to the disclosure were tested for their ability to diagnose cancer in test cell samples isolated from human subjects suffering from ovarian cancer, lung cancer, prostate cancer, hepatic cancer, pancreatic cancer, breast cancer, leukemia, sarcoma, melanoma, renal cancer, colon cancer, and osteosarchma. The expression levels of SLC7A5 RNA and SLC7A5 protein in combination with other cancer markers were analyzed for differential expression in lung, breast, ovarian, colon and prostate samples by Real-time PCR and Western blot. The testing and results are described in detail below in the Examples, and the results are summarized below.

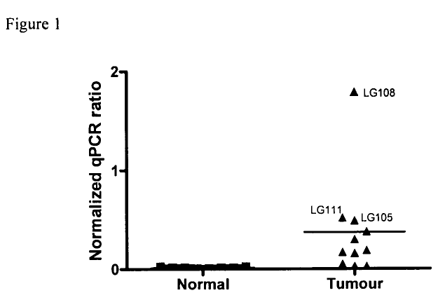

SLC7A5 RNA expression is increased in lung tumor tissues as compared to normal lung tissues (FIG. 1). These results indicate that the increase in SLC7A5 expression is a marker of the transformation of normal lung cells to neoplastic lung cells.

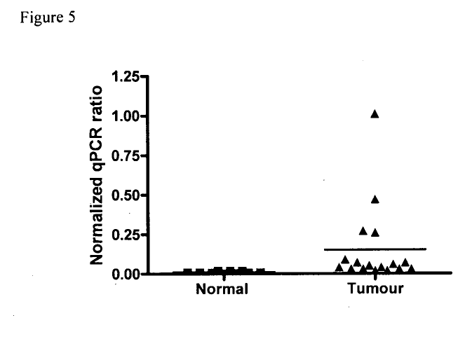

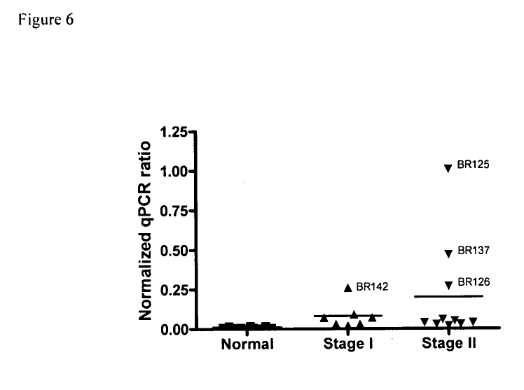

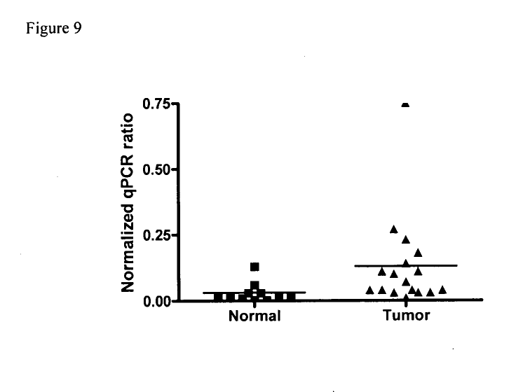

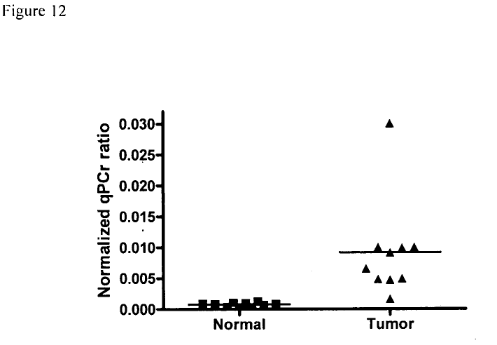

Increased expression of TTK RNA was also observed in breast cancer patient samples as compared to normal tissue samples (FIGS. 5 and 6). In addition, ovarian cancer samples showed higher levels of RNA expression as compared to normal ovarian tissues (FIG. 9). Similarly, SLC7A5 RNA expression was increased in colorectal cancer sample versus normal colon tissue from patients (FIG. 12). TTK RNA expression did not show a significant increase in Stage I prostate cancer samples when compared to normal prostate tissue samples.

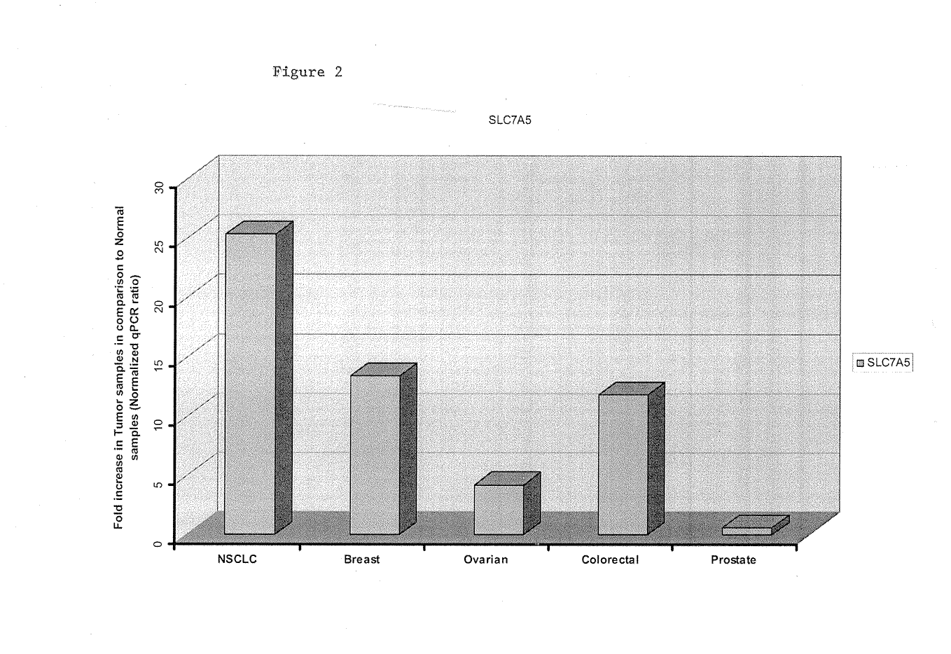

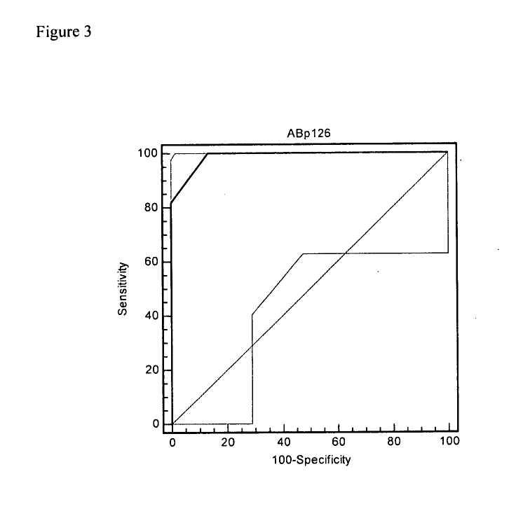

FIG. 2 and Table 1 summarize the results of the RNA experiments by showing the normalized Real-time PCR ratios of SLC7A5 expression levels found in lung (NSCLC), breast, ovarian, colorectal and prostate cancer patients and normal tissue subjects. The results shown in this figure are based on sample size of NSCLC (n=15N+11T); Breast (N=10N+17T); Ovarian (n=10N+17T); Colorectal (n=10N+10T matched); Prostate (n=10N+10T matched).

In summary, RNA expression lung, breast, ovarian, and colon studies show that SLC7A5 is a marker of the transformation of normal cells to neoplastic cells of the same lineage.

TABLE 1

| Cancer type | SLC7A5 |
|---|---|
| NSCLC | 25.3 |
| Breast | 13.4 |
| Ovarian | 4.2 |
| Colorectal | 11.8 |
| Prostate | 0.64 |

Table 2 shows a compilation of SLC7A5 results in cell lines from various cancers as compared with tissue matched controls.

TABLE 2

| | | SLC7A5 |
|---|---|---|
| Cancer | | expression |
| type | Cell lines | level |
| Breast | MCF7 | 75.40 |
| | MDA | 8.68 |
| Ovarian | SKOV3 | 19.21 |
| | 2008 | 57.85 |
| | OVCAR-3 | 8.58 |
| Colorectal | T84 | 25.26 |
| | HCT116 | 96.09 |
| Lung | H460 | 169.04 |
| | A549 | 56.82 |
| Prostate | PC3 | 73.84 |

Other markers were also tested for differential expression in lung, breast, ovarian, colorectal and prostate tissues. There is significant increase in TTK, KIF20A, TRIM59 and UHRF1 RNA expression in lung (NSCLC) cancer versus normal lung tissues. Similar increase in RNA expression of TTK, KIF20A, TRIM59 and UHRF1 is seen in breast, ovarian, and colorectal cancers versus normal tissues for each respective cancer. These results indicate that these proteins can be used as markers of certain neoplastic disease in combination with SLC7A5.

Table 3 shows a compilation of the RNA expression results found in lung, breast, ovarian, and colorectal cancer tissues as compared to tissue-matched controls, together with the quantified fold increases for TTK, SLC7A5, TRIM59, UHRF1 and KIF20A RNAs.

TABLE 3

| | Breast | | Ovarian | | | Colon | | Lung | |
|---|---|---|---|---|---|---|---|---|---|
| ABP Biomarkers | MCF7 | MDA | SKOV3 | 2008 | OVCAR3 | T84 | HCT116 | H460 | A549 |
| TTK | 157.7 | 2777.8 | 14.3 | 19.2 | 37.7 | 38.1 | 6.1 | 215 | 15.4 |
| SLC | 75.4 | 8.7 | 19.2 | 57.9 | 8.6 | 25.2 | 96.1 | 169 | 56.8 |
| TRIM59 | 6.9 | 9.5 | 12.7 | 8.6 | 28.9 | 11.8 | 9.2 | 8 | 45.1 |
| KIF20A | 18.7 | 36.7 | 11.3 | 5.6 | 16.2 | 19.2 | 9.1 | 53.9 | 29.1 |
| UHRF1 | 8.6 | 30.5 | 8.7 | 5.6 | 5.2 | 8.8 | 8.4 | 74.9 | 68.5 |

In all, these results, in combination with the results described in the examples, indicate that UHRF1 alone, or in combination with TRIM59, TTK, UHRF1, and/or KIF20A described herein, is a marker of certain neoplastic disease.

### 1.9 Binding Conjugates Used for Treatment

In addition to detectable moieties, other non-limiting moieties that may be operably linked to a binding agent of the disclosure include, without limitation, a toxin, an enzyme, an antibody (or a portion thereof), a cytotoxic drug, or a conjugate thereof or mixture thereof. Where a toxin is operably linked to a binding agent of the disclosure, non-limiting examples of a toxin which can be operably linked to a binding agent of the disclosure include a radioactive isotope, Diptheria toxin, a nuclease (e.g., DNAse or RNAse), a protease, a degradative enzyme, Pseudomonas exotoxin (PE), ricin A or B chains, and ribonuclease A (Fizgerald D., Semin. Cancer Biol. 7: 87-95, 1996).

In some embodiments, the binding agent is an immunotoxin (e.g., an antibody-toxin conjugate or antibody-drug conjugate). Non-limiting examples of immunotoxins include antibody-anthracycline conjugates (Braslawsky, et al., EP 0398305), antibody-cytokine conjugates (Gilles, PCT WO9953958), and monoclonal antibody-PE conjugates (Roffler, et al., Cancer Res. 51: 4001-4007, 1991).

In a further aspect, the disclosure provides a therapeutic composition comprising a cytotoxic drug, a binding agent that specifically binds to a SLC7A5 protein or fragment thereof, and a pharmaceutically-acceptable carrier. Non-limiting examples of such pharmaceutically-acceptable carriers are described in more detail in Remington: The Science and Practice of Pharmacy, Gennaro et al. (eds), 20^{th} Edition, Lippincott Williams & Wilkins, Philadelphia, Pa., 2001 (ISBN 0-683-306472), a standard reference text. In certain embodiments, binding of the binding agent is toxic to damaged cells, regardless of whether such cells are drug-sensitive. In some embodiments, binding of the binding agent is toxic to neoplastic cells, regardless of whether such cells are drug-sensitive. In certain embodiments, the binding agent of the composition is operably linked to a toxin.

Actual methods for preparing therapeutic compositions are known or apparent to those skilled in the art, and are described in detail in Remington: The Science and Practice of Pharmacy, 2001 (supra); and in Pharmaceutical Dosage Forms and Drug Delivery Systems, 6th ed., Williams & Wilkins (1995). The therapeutic compositions of the disclosure may be in any form suitable for administration including, without limitation, in the form of a tablet, a capsule, a powder, a solution, or an elixir.

Note that a cytotoxic drug of the therapeutic composition of the disclosure need not be cytotoxic to all cells. In some embodiments, where the therapeutic composition is being administered to a patient suffering from a disease caused by the presence of a damaged cell, the cytotoxic drug of the therapeutic composition is an antipathogenic or anti-microbial drug. In some embodiments, where the damaged cells are infected with a pathogen (e.g., a virus, a bacterium, or a multi-cellular parasite) and the disease is caused by the infection, the cytotoxic drug of the therapeutic composition is an antipathogenic drug. Where the damaged cells are infected by a pathogen, non-limiting examples of the drug which differs depending upon the infecting pathogen include, but are not limited to, Acyclovir, amphotericin, ampicillin, anthracyclin, b-lactam antibiotics, cephalothin, chloramphenicol, chloroquine (CQ), cidofovir (CDV), ciprofloxacin, erythromycin, fluconazole, 5 flucytosine, fluoroquinolone, foscarnet, gancyclovir, halofantrine, Itraconazole, lamivudine, macrolides, mefloquine, methicillin, metronidazole, miconazole, nelfinavir, ofloxacin, penicillin, primaquine, quinoline, Streptomycin, Sulfonamides, teicoplanin, terbinafine, tetracycline, vancomycin, voriconazole. Therapeutically effective amounts of such drugs are known to routinely skilled physicians and pharmacists. In addition, such information can be obtained from the manufacturer of the drug, or from the Physician's Desk Reference, Medical Economics Co. (published yearly).

In some embodiments, where the therapeutic composition is being administered to a patient suffering from a cancer caused by the presence of a neoplastic cell, the cytotoxic drug of the therapeutic composition is an anti-cancer drug. Such anti-cancer drugs include, without limitation, chemotherapeutic drugs and radiotherapeutic drugs. Non-limiting examples of such anti-cancer drugs include Actinomycin, Adriamycin (AR), Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin (DOX), Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin (MITO), Mitotane, Mitoxantrone, Paclitaxel, Pentostatin, Procarbazine, Taxol, Teniposide, Topotecan, Vinblastine (VLB), Vincristine, and Vinorelbine. Therapeutically effective amounts of such drugs are known to routinely skilled physicians and pharmacists. In addition, such information can be obtained from the manufacturer of the drug, or from the Physician's Desk Reference, Medical Economics Co. (published yearly).

In another aspect, the disclosure provides a method for treating a patient suffering from a disease caused by the presence of damaged cells. This method includes administering to the patient a therapeutically effective amount of a drug and a therapeutically effective amount of a binding agent that specifically binds to a SLC7A5 protein or fragment thereof. Of course, since the SLC7A5 protein is expressed at moderate levels on drug-sensitive damaged cells, the binding agent, which, in some embodiments is different than the drug, will also kill drug-sensitive damaged cells. According to this method, the patient shows an improved prognosis for the disease as compared to an untreated patient. In some embodiments, the untreated patient receives no binding agent, but does receive the drug. An “untreated patient” or “control patient” is one that receives no binding agent, but does receive the drug; or one that receives no treatment at all (i.e., receives neither the binding agent nor the drug). The drug and the binding agent can be separately administered at different times in any order or can be administered together. In some embodiments, the patient is a human.

In certain embodiments, the damaged cells of the patient are infected with a pathogen. In particular embodiments, the pathogen is a virus, a bacterium, or a parasite (HIV, West Nile virus and Dengue virus; Mycobacteria, Rickettsia, and Chlamydia; Plasmodium, Leishmania, and Taxoplasma)

In another aspect, the disclosure provides a method for treating a patient suffering from a disease (e.g., cancer) caused by the presence of neoplastic cells. This method includes administering to the patient a therapeutically effective amount of a drug and a therapeutically effective amount of a binding agent that specifically binds to a SLC7A5 protein or fragment thereof. The protein is expressed at low levels on the surface of normal tissues, but at much higher levels on the surface of neoplastic cells. Since the SLC7A5 protein is expressed at higher levels (see Table 1), or neoplastic cells, in some embodiments, the binding agent (which, in some embodiments is different than the drug) also kills neoplastic cells. According to this method, the patient shows an improved prognosis for the disease as compared to an untreated patient. In some embodiments, the untreated patient receives no binding agent, but does receive the drug. In some embodiments, the untreated patient receives no treatment at all (i.e., receives neither the binding agent nor the drug). The drug and the binding agent (e.g., an antibody) can be separately administered in any order at different times or can be administered together. In some embodiments, the patient is a human.

In certain embodiments, the neoplastic cells of the patient are breast cancer cells, ovarian cancer cells, myeloma cancer cells, lymphoma cancer cells, melanoma cancer cells, sarcoma cancer cells, leukemia cancer cells, retinoblastoma cancer cells, hepatoma cancer cells, glioma cancer cells, mesothelioma cancer cells, or carcinoma cancer cells.

In some embodiments, the binding agent is operably linked to a toxin. Non-limiting examples of such toxins are described above.

As used herein, the term “therapeutically effective amount” is used to denote known treatments of a drug at dosages and for periods of time effective to kill a damaged cell. Administration may be by any route including, without limitation, intravenous, parenteral, oral, sublingual, transdermal, topical, intranasal, intraocular, intravaginal, intrarectal, intraarterial, intramuscular, subcutaneous, and intraperitoneal.

The dose and dosage regimen of a binding agent, drug, and/or therapeutic composition in accordance with the disclosure, will depend mainly on the degree of symptoms of the disease or cancer, the type of drug used (e.g., chemotherapeutic agent, radiotherapeutic agent, or antibiotic), the patient (e.g., the patient's gender, age, and/or weight), the patient's history, and the patient's response to treatment. The doses of binding agent, drug, and/or therapeutic composition may be single doses or multiple doses. If multiple doses are employed, the frequency of administration (schedule) will depend, for example, on the patient, type of response, and type of drug used. Administration once a week may be effective for some patients; whereas for others, daily administration or administration every other day or every third day may be effective. The practitioner will be able to ascertain upon routine experimentation, which route of administration and frequency of administration are most effective in any particular case.

In certain embodiments, the patient is a mammal such as a human. The patient may be, for example, a human suffering from a disease caused by the presence of the neoplastic or damaged cell. For example, the patient may be suffering from cancer. Such a neoplastic cell includes, without limitation, an ovarian cancer cell, and myeloma cancer cell, a lymphoma cancer cell, a melanoma cancer cell, a sarcoma cancer cell, a leukemia cancer cell, a retinoblastoma cancer cell a hepatoma cancer cell, a glioma cancer cell, a mesothelioma cancer cell, or a carcinoma cancer cell.

In some embodiments, the cell is a damaged cell, and the patient is suffering from a disease caused by the presence of such a damaged cell. Non-limiting ways in which a cell may be damaged include infection by a pathogen (e.g., virus, bacteria or parasite), or a cell may suffer damage by necrosis. In particular embodiments, the damaged cell is infected with a pathogen (e.g., a virus, parasite, or bacterium).

The disclosure provides antibodies directed against SLC7A5 for use in detection, imaging and treatment of cancers and damaged (e.g., pathogen-infected) cells. Anti-SLC7A5 antibodies for use in the disclosure are available from several commercial vendors.

The anti-SLC7A5 antibodies of the disclosure can be administered orally or parenterally subcutaneously, intramuscularly, intravenously or interperitoneally.

The antibodies, antibody fragments, mixtures or derivatives thereof can be used in therapy or diagnosis directly or after coupling to solid or liquid carriers, enzymes, toxins, radioactive or nonradioactive labels or to fluorescent/chemiluminescent labels as described above. SLC7A5 can be detected on a wide variety of cell types—particularly neoplastic cells. The human SLC7A5 monoclonal antibody of the present disclosure may be obtained as follows. Those of skill in the art will recognize that other equivalent procedures for obtaining SLC7A5 antibodies are also available and are included in the disclosure.

First, a mammal is immunized with human SLC7A5. Purified human SLC7A5 is commercially available from Sigma (St. Louis, Mo., catalog A6152), as well as other commercial vendors. Human SLC7A5 may be readily purified from human placental tissue. Furthermore, methods of immunoaffinity purification for obtaining highly purified SLC7A5 immunogen are known (see, e.g., Vladutiu et al., (1975) Prep. Biochem. 5: 147-59). The mammal used for raising anti-human SLC7A5 antibody is not restricted and may be a primate, a rodent such as mouse, rat or rabbit, bovine, sheep, goat or dog.

Next, antibody-producing cells such as spleen cells are removed from the immunized animal and are fused with myeloma cells. The myeloma cells are well-known in the art (e.g., p3×63-Ag8-653, NS-0, NS-1 or P3U1 cells may be used). The cell fusion operation may be carried out by a well-known conventional method.

The cells, after being subjected to the cell fusion operation, are then cultured in HAT selection medium so as to select hybridomas. Hybridomas, which produce antihuman monoclonal antibodies, are then screened. This screening may be carried out by, for example, sandwich ELISA (enzyme-linked immunosorbent assay) or the like in which the produced monoclonal antibodies are bound to the wells to which human SLC7A5 is immobilized. In this case, as the secondary antibody, an antibody specific to the immunoglobulin of the immunized animal, which is labeled with an enzyme such as peroxidase, alkaline phosphatase, glucose oxidase, beta-D-galactosidase or the like, may be employed. The label may be detected by reacting the labeling enzyme with its substrate and measuring the generated color. As the substrate, 3,3-diaminobenzidine, 2,2-diaminobis-o-dianisidine, 4-chloronaphthol, 4-aminoantipyrine, o-phenylenediamine or the like may be produced.

By the above-described operation, hybridomas, which produce anti-human SLC7A5 antibodies, can be selected. The selected hybridomas are then cloned by the conventional limiting dilution method or soft agar method. If desired, the cloned hybridomas may be cultured on a large scale using a serum-containing or a serum free medium, or may be inoculated into the abdominal cavity of mice and recovered from ascites, thereby a large number of the cloned hybridomas may be obtained.

From among the selected anti-human SLC7A5 monoclonal antibodies, those that have an ability to bind cell surface SLC7A5 are then chosen for further analysis and manipulation.

The monoclonal antibodies herein further include hybrid and recombinant antibodies produced by splicing a variable (including hypervariable) domain of an anti-SLC7A5 antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)).

“Humanized” forms of non-human (e.g., murine) antibodies are specific chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin.

Human antibodies directed against SLC7A5 are also useful in the methods of the disclosure. Such antibodies can be made, for example, by the hybridoma method. Human myeloma and mouse-human heteromyeloma cell lines for the production of human monoclonal antibodies have been described, for example, by Kozbor (1984) J. Immunol., 133, 3001; Brodeur, et al., Monoclonal Antibody Production Techniques and Applications, pp. 51-63 (Marcel Dekker, Inc., New York, 1987); and Boerner et al., (1991) J. Immunol., 147:86-95. Specific methods for the generation of such human antibodies using, for example, phage display, transgenic mouse technologies and/or in vitro display technologies, such as ribosome display or covalent display, have been described (see Osbourn et al. (2003) Drug Discov. Today 8: 845-51; Maynard and Georgiou (2000) Ann. Rev. Biomed. Eng. 2: 339-76; and U.S. Pat. Nos. 4,833,077; 5,811,524; 5,958,765; 6,413,771; and 6,537,809.

Transgenic animals can be produced (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such gem-line mutant mice will result in the production of human antibodies upon antigen challenge (see, e.g., Jakobovits et al., (1993) Proc. Natl. Acad. Sci. (USA), 90: 2551; Jakobovits et al., (1993) Nature, 362:255-258; and Bruggermann et al., (1993) Year in Immuno., 7:33).

Alternatively, phage display technology (McCafferty et al., (1990) Nature, 348: 552-553) can be used to produce human antibodies and antibody fragments in vitro, from immunoglobulin variable (V) domain gene repertoires from unimmunized donors.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (see Marks et al., (1992) Bio/Technol., 10:779-783).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published 1 Apr. 1993). Unlike traditional humanization of rodent antibodies by CDR grafting, this technique provides completely human antibodies, which have no framework or CDR residues of rodent origin.

### 1.10 SLC7A5-Targeted Therapeutics

The disclosure takes advantage of the fact that SLC7A5 protein cell surface marker is present only in negligible levels on the surface of normal cells of the body, but occurs (at high levels) on the cell surface of neoplastic cells. In contrast, other markers are present at variable levels on the surface of many different normal cell and tissues, including high levels on the surface of liver, kidney, stem cells, and blood-brain barrier epithelial cells. Accordingly, the disclosure provides a highly specific way of targeting therapeutics to neoplastic and damaged cells using a binding agent that binds to cell surface SLC7A5.

Therapeutic agents to be targeted to SLC7A5 by the methods of the disclosure include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Anti-SLC7A5 Antibodies

In one approach, anti-SLC7A5 antibodies specific to cell surface expressed SLC7A5 expressed on damaged (e.g., pathogen-infected), neoplastic cells are administered systemically to a patient with cancer. Adhesion of antibody to tumor cells results in tumor cell death by activation of the complement system (complement-mediated cytotoxicity) or by activation of T cells (antibody-dependent cell-mediated cytotoxicity). Other antibody-induced antitumor effects include induction of apoptosis, enhancement of the cytotoxic effects of a second agent (e.g., an anti-cancer chemotherapeutic drug), and induction of anti-idiotype network response. In certain embodiments, humanized anti-SLC7A5 antibodies may be utilized. Humanized antibodies avoid the potential problem of causing human patients to develop anti-animal (e.g., anti-mouse or anti-rat) antibodies. Humanized antibodies consist of human antibody contain the complementarily-determining region from a nonhuman source.

SLC7A5-Targeted Antibody and Ligand Conjugates

In addition to the ‘naked’ antibody approach described above, antibodies can be conjugated, or otherwise “operably linked” to biological or chemical toxins or radioisotopes. An anti-SLC7A5 antibody or antibody fragment thereof may be conjugated or otherwise operably linked to a therapeutic moiety such as a cytotoxin, e.g., a cytostatic or cytocidal agent, a therapeutic agent or a radioactive metal ion. “Operably linked” means that the therapeutic moiety is attached to the binding agent by either a covalent or non-covalent (e.g., hydrophobic or ionic) bond. Methods for creating covalent bonds are known (see general protocols in, e.g., Wong, Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y. 1999). Following systemic administration, the therapy is targeted to the cancer cell or damaged cell by the antibody.

The disclosure further includes SLC7A5-targeted agents made up of a SLC7A5 targeting element and a toxic agent, such as a biological toxin, a chemical toxin or a radioisotope. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Biological toxins have been conjugated, or genetically fused in frame, to antibodies, and other tumor marker-localizing agents. These biological toxins include ricin, diphtheria toxin and Pseudomonas exotoxin. Following binding to cell surface SLC7A5, the toxins generally cross the cell membrane, and may then be processed, before killing the targeted cell. The toxic effect is typically due to inhibition of protein-synthesis by the active biological toxin.

For example, in one embodiment, anti-SLC7A5 antibodies are conjugated to cobra venom factor. In accordance with the disclosure, SLC7A5 specific antibodies conjugated to cobra venom factor are used to treat cancer in a human. Methods of conjugating antibodies to cobra venom factor are taught in U.S. Pat. No. 5,773,243. In some embodiments, the binding agent is an immunotoxin (e.g., an antibody-toxin conjugate or antibody-drug conjugate). Non-limiting examples of immunotoxins include antibody-anthracycline conjugates (Braslawsky G. R. et al., European Patent No. EP0398305), antibody-cytokine conjugates (Gilles, PCT WO9953958), and monoclonal antibody-PE conjugates (Roffler, et al., Cancer Res. 51: 4001-4007, 1991).

Techniques for conjugating other therapeutic moieties to antibodies are well known, see, e.g., Arnon et al., in Monoclonal Antibodies and Cancer Therapy, Reisfeld et al. (eds.), pp. 243-56 (Alan R. Liss, Inc. 1985); Hellstrom et al., in Controlled Drug Delivery (2nd Ed.), Robinson et al. (eds.), pp. 623-53 (Marcel Dekker, Inc. 1987); Thorpe, in Monoclonal Antibodies '84: Biological And Clinical Applications, Pinchera et al. (eds.), pp. 475-506 (1985); in Monoclonal Antibodies For Cancer Detection and Therapy, Baldwin et al. (eds.), pp. 303-16 (Academic Press 1985), and Thorpe et al., Immunol. Rev., 62:119-58 (1982); each of which is incorporated herein by reference. Alternatively, an antibody can be conjugated to a second antibody to form an antibody heteroconjugate as described by Segal in U.S. Pat. No. 4,676,980, which is incorporated herein by reference.

Drug Attachment

A number of approaches to drug and therapeutics attachment and release are known (see, e.g., Soyez, et al., (1966) Adv. Drug Del. Rev. 21:81-106); Ref. Wong (1991) CRC Press, Boca Raton, Fla.).

For example, ε-amino groups of the lysine residues are chemically convenient to use, either by amide bond forming reagents such as carbodiimides or by heterobifunctional agents such as N-succinimidyl-3-(2-pyridyldithio) propionate (SPDP) (Carlsson, et al., (1978) Biochem J. 173:723-737) which can introduce reactive thiol groups. Antibodies have a variable number of lysine residues, which are spread over the whole of the antibody, and there is no evidence for any subset of more reactive residues.

A second site for modification is the sugar residues attached to the hinge region of the antibody. (see, e.g., O'Shanessy, et al., (1984) Immunol. Lett. 8:273-277; O'Shanessy, et al., (1987) J. Immunol. Methods 99:153-161; and Rodwell, et al., (1986) Proc. Natl. Acad. Sci. USA 83:2632-2636). Aldehyde groups can also be generated on immunoglobulins by an enzymic reaction involving neuraminidase and glucose oxidase (Rodwell, et al., (1986) Proc. Natl. Acad. Sci. USA 83:2632-2636 and Stan, et al., (1999) Cancer Res. 59:115-121). Antibodies have also been modified by genetic engineering to produce new oligosaccharides sites which are reported to be more favourably located for attachment of carrier-drug molecules (Qu, et al., (1998) J. Immunol. Methods 213:131-144).

The third major possibility for attachment is through internal disulphide bonds within the antibody (see, e.g., Willner et al., (1993) Bioconjug. Chem. 4:521-527).

Acid-labile linkages may also be used to link therapeutic agents to antibodies or other localizing agents. Chemically labile linkages can be used to release drug in the presence of more acid conditions. These conditions can occur either in the tumour environment which is reported to be 0.5-1 pH unit more acidic than health tissue and blood (Lavie, et al., (1991) Cancer Immunol. Immunther. 33:223-230 and Ashby (1966) Lancet ii:312-315), or during passage through the endosomal/lysosomal compartment, where pH of 6-6.8 and 4.5-5.5, respectively, can be found.

Enzymically degradable linkers may also be used to link therapeutic agents to antibodies or other localizing agents. The gold standard for attaching and releasing drugs from macromolecules is a linker which is stable in serum but can be cleaved intracellularly by specific enzymes. Linkers of this type have been described containing a variety of amino acids. Some of these linkers have been used in targeted drug conjugates with antibodies, but others only in polymer-drug conjugates.

Generally the simplest way of producing an immunoconjugate is to couple the drug directly to the antibody. This involves a direct linkage between the functional group of the drug, and one of the functional groups on the antibody, or alternatively may involve the interposition of a linker or spacer group between these two parts of the conjugate. A linker group may be used merely to make the chemistry of the coupling possible, but may have the second function of allowing a specific type of release of the drug. If the release is mediated by an enzyme, located either intra- or extracellularly, the group may be termed a spacer group, its purpose being to allow sufficient space, or reduce steric constraints so that the enzyme can access the relevant bond adequately.

The conjugates that have been produced have been documented in many reviews (e.g., Magerstädt (1991) CRC Press Boca Raton, Fla. 77-215; Dubowchik, et al., (1999) Pharmacol. Ther. 83:67-123; and Pietersz, et al., (1994) Adv. Immunol. 56:301-387).

Antibody concentration is an important determinant of the rate of drug uptake; therefore, if more drug molecules can be conjugated per antibody molecule, cytotoxicity should increase.

Another solution to the difficulties of delivering sufficient drug molecules to kill cancer cells is to use more potent drugs, which require fewer molecules of drug to kill a cell. A number of these molecules are known and include: CC-1065-like alkylating agents such as Duocarmycin; Enediynes, including the dynemicins, the calicheamicins/esperamicins, and the chromoproteins (Borders, et al., (1994) Marcel Dekker New York) (e.g., Neocarzinostatin, and Calicheamicin), and Macrolide antibiotics such as Geldanamycin and maytansine.

Immuntoxins

The therapeutic agent or drug moiety is not to be construed as limited to classical chemical or radiological therapeutic agents. For example, the drug moiety may be a protein or polypeptide possessing a desired biological activity. Such proteins may include, for example, in addition to toxins such as abrin, ricin A, pseudomonas exotoxin, or diphtheria toxin, other proteins with biological activity such as tumor necrosis factor, alpha-interferon, beta-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, IL-1, IL-2, IL-4, IL-5, IL-6, IL-7, 11-8, 11-9, 11-10, IL-12, IL-15, granulocyte macrophase colony stimulating factor (“GM-CSF”), granulocyte colony stimulating factor (“G-CSF”), or other growth factors.

Immunotoxins contain a ligand such as a growth factor, monoclonal antibody, or fragment of an antibody which is connected to a protein toxin. After the ligand subunit binds to the surface of the target cell, the molecule internalizes and the toxin kills the cell. Bacterial toxins which have been targeted to cancer cells include Pseudomonas exotoxin and diphtheria toxin, which are well suited to forming recombinant single-chain or double-chain fusion toxins. Plant toxins include ricin, abrin, pokeweed antiviral protein, saporin and gelonin, and have generally been connected to ligands by disulfide-bond chemistry. Immunotoxins have been produced to target hematologic malignancies and solid tumors via wide variety of growth factor receptors and antigens.

One goal of immunotoxin therapy is to target a cytotoxic agent to cell surface molecules which will internalize the cytotoxic agent and result in cell death. Since immunotoxins differ greatly from chemotherapy in their mode of action and toxicity profile, immunotoxins provide improved systemic treatment of tumors.

Immunotoxins can be defined as proteins containing a toxin and an antibody. Toxins reviewed here include catalytic proteins produced by plants or bacteria which kill target cells. While the term ‘immunotoxin’ generally refers to a toxin targeted by either an intact IgG, an Fab fragment or an Fv fragment, toxins targeted by growth factors or other ligands are also referred to as ‘chimeric toxins’. In some immunotoxins or chimeric toxins, the linkage between the ligand and the toxin is made chemically, and the proteins may be referred to as ‘chemical conjugates’. Otherwise, when the linkage is a peptide bond produced by genetic engineering, the proteins may be referred to as ‘recombinant toxins’ or ‘fusion toxins’. Finally, a select group of immunotoxins contain an Fv sequence fused to the toxin, and these proteins, being both immunotoxins and recombinant toxins, are often referred to as ‘recombinant immunotoxins’.

Plant toxins exist in nature as holotoxins and hemitoxins. Holotoxins (also referred to as class II ribosome in activating proteins) include ricin, abrin, misdetoe lectin and modeccin, which contain a binding domain disulfide-bonded to an enzymatic domain. Hemitoxins, such as pokeweed antiviral protein (PAP), saporin and gelonin contain an enzymatic but no binding domain.

To make immunotoxins, plant toxins are generally conjugated chemically to ligands (see e.g., Kreitman, et al., (1998) Adv. Drug Del. Rev., 31:53-88).

Two bacterial toxins that have been used to make immunotoxins include Pseudomonas exotoxin (PE), made by Pseudomonas aeruginosa, and diphtheria toxin (DT), made by Corynebacterium diphtherae. Both PE and DT catalytically ADP ribosylate EF-2 in the cytosol (see Carroll et al., (1987) J. Biol. Chem. 262:8707-8711; Uchida et al., (1972) Science 175:901-903; and Uchida et al., (1973) J. Biol. Chem. 248:3838-3844). Mutated and truncated forms of DT and PE may also be used (see Kreitman, et al., (1998) Adv. Drug Del. Rev., 31:53-88).

Toxins can be targeted to cells without chemically conjugating the ligand and the toxin if both are connected as one polypeptide unit.

Targeted Radiotherapy

Radioisotopes may also be used as cytotoxic agents for SLC7A5-targeted therapeutics. Anti-SLC7A5 antibodies of the present disclosure may be coupled to one or more therapeutic agents. Suitable agents in this regard include radionuclides. Suitable radionuclides include ⁹⁰Y, ¹²³I, ¹²⁵I, ¹³¹I, ¹⁸⁶Re, ¹⁸⁸Re, ²¹¹At, and ²¹²Bi. Carriers specific for radionuclide agents, to facilitate attachment to the SLC7A5 targeting agent, include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

An ideal radioligand therapy agent accumulates selectively in target cells. The effectiveness of radiotherapy is due to the destruction of dividing cells resulting from radiation-induced damage to cellular DNA (see, e.g., Bloomer et al., (1977)).

Radioisotopes suitable for therapeutic treatment include Auger-electron-emitting radioisotopes, e.g. ¹²⁵I, ¹²³I, ¹²⁴I, ¹²⁹I, ¹³¹I, ¹¹¹In, ⁷⁷Br, and other radiolabeled halogens. The choice of a suitable radioisotope can be optimized based on a variety of factors including the type of radiation emitted, the emission energies, the distance over which energy is deposited, and the physical half-life of the radioisotope. In certain instances, the radioisotopes used are those having a radioactive half-life corresponding to, or longer than, the biological half-life of the SLC7A5-targeted therapeutic. For example, in certain examples the radioisotope has a half-life between about 1 hour and 60 days, or between 5 hours and 60 days, or between 12 hours and 60 days. ¹²⁵I has an advantage over other emitters that produce high-energy gamma rays (i.e., ¹¹¹In, and ¹³¹I) which require inpatient hospitalization and isolation ¹²⁵I will allow the development of outpatient-based treatments due to the limited amounts of radiation that escapes the body.

Radiolabeled therapeutics have typically been administered by intravenous, bolus injection (see, e.g., Kalofonos et al., (1989); J. Nucl. Med. 30:163-645; Virgolini et al., (1994) N. Eng. J. Med., 331:1116-21).

Targeted Gene Therapy

Gene vectors may also be used as cytotoxic agents for SLC7A5-targeted therapeutics. For example, a gene vector encoding an antibody gene (or fragment thereof) inside the tumor cell. The transgene expression product binds intracellular proteins, e.g., those derived from oncogenes, and thereby down-regulates oncogenic protein expression. Targeted gene therapy may be facilitated by the use of bifunctional crosslinkers to target adenoviral and retroviral vectors, by inserting short targeting peptides and larger polypeptide-binding domains into the coat protein of a number of different viral vectors, and by the use of replication-competent vectors (see Wand, et al. (2003) Act. Biochim. Biophys. Sinica 35(4): 311-6). Other non-viral therapeutic agents, including DNA complexes and bacterial vehicles, have also been developed. Gene therapy methods for SLC7A5-targeted compositions and methods of the disclosure may be adapted from gene therapy methods known in the art or adapted from U.S. Pat. Nos. 5,871,726, 5,885,806, 5,888,767, 5,981,274, 6,207,426, 6,210,708, 6,232,120, 6,498,033, 6,537,805, 6,555,107, and 6,569,426.

In one approach, targeted replicative or non-replicative viral vectors may be used to deliver the gene therapeutic. For example, andoviral gene therapy vectors have been adapted for the targeting of neoplastic cells (see Rots, et al. (2003) J. Controlled Release 87: 159-165). Selective targeting of adenovirus vectors limits the inflammatory and immune response against the viral vector and decreases the toxicity of the treatment because lower doses of virus can be used.

In general, the terms “viral vectors” and “viruses” are used interchangeably herein to refer to any of the obligate intracellular parasites having no protein-synthesizing or energy-generating mechanism. The viral genome may be RNA or DNA contained with a coated structure of protein of a lipid membrane. The terms virus(es) and viral vector(s) are used interchangeably herein. The viruses useful in the practice of the present disclosure include recombinantly modified enveloped or non-enveloped DNA and RNA viruses, selected from baculoviridiae, parvoviridiae, picornoviridiae, herpesviridiae, poxyiridae, or adenoviridiae. The viruses may be naturally occurring viruses or their viral genomes may be modified by recombinant DNA techniques to include expression of exogenous transgenes and may be engineered to be replication deficient, conditionally replicating or replication competent. Chimeric viral vectors which exploit advantageous elements of each of the parent vector properties (See e.g., Feng, et al. (1997) Nat. Biotechnol. 15:866-870) may also be useful in the practice of the present disclosure. Minimal vector systems in which the viral backbone contains only the sequences need for packaging of the viral vector and may optionally include a transgene expression cassette may also be produced according to the practice of the present disclosure. Although it is generally favored to employ a virus from the species to be treated, in some instances it may be advantageous to use vectors derived from different species that possess favorable pathogenic features. For example, equine herpes virus vectors for human gene therapy are described in WO98/27216 published Aug. 5, 1998. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in WO 97/06826 published Apr. 10, 1997.

The term “replication deficient” refers to vectors which are incapable of replication in a wild type mammalian cell. In order to produce such vectors in quantity, the producer cell line must be cotransfected with a helper virus or modified to complement the missing functions. For example, 293 cells have been engineered to complement adenoviral E1 deletions allowing propagation of the E1 deleted replication deficient adenoviral vectors in 293 cells. The term “replication competent viral vectors” refers to a viral vector which is capable of infection, DNA replication, packaging and lysis of an infected cell. The term “conditionally replicating viral vectors” is used herein to refer to replication competent vectors which are designed to achieve selective expression in particular cell types while avoiding untoward broad spectrum infection. Such conditional replication may be achieved by operably linking tissue specific, tumor specific or cell type specific or other selectively induced regulatory control sequences to early genes (e.g. the E1 gene of adenoviral vectors).

In addition to targeting, cell type specificity with viral vectors may be improved through the use of a pathway responsive promoters driving a repressor of viral replication. The term “pathway-responsive promoter” refers to DNA sequences that bind a certain protein and cause nearby genes to respond transcriptionally to the binding of the protein in normal cells. Such promoters may be generated by incorporating response elements which are sequences to which transcription factors bind. Such responses are generally inductive, though there are several cases where increasing protein levels decrease transcription. Pathway-responsive promoters may be naturally occurring or synthetic.

In the certain applications of the disclosure, the viral vector is an adenovirus. The term “adenovirus” is synonomous with the term “adenoviral vector” and refers to viruses of the genus adenoviridiae. The term adenoviridiae refers collectively to animal adenoviruses of the genus mastadenovirus including but no limited to human, bovine, ovine, equine, canine, porcine, murine and simian adenovirus subgenera.

The therapeutic gene to be delivered is generally a cytotoxic gene, a tumor suppressor gene, a toxin gene, a pro-apoptotic gene, a pro-drug activating gene, or a cytokine gene. The term “cytotoxic transgene” refers to a nucleotide sequence the expression of which in the target cell induces lysis or apoptosis of the cell. The term “cytotoxic transgene” includes but is not limited to tumor suppressor genes, toxin genes, cytostatic genes, pro-drug activating genes, or apoptotic genes. The vectors of the present disclosure may be used to produce one or more therapeutic transgenes, either in tandem through the use of IRES elements or through independently regulated promoters.

The term “tumor suppressor gene” refers to a nucleotide sequence, the expression of which in the target cell is capable of suppressing the neoplastic phenotype and/or inducing apoptosis. Examples of tumor suppressor genes useful in the practice of the present disclosure include the p53 gene, the APC gene, the DPC-4 gene, the BRCA-1 gene, the BRCA-2 gene, the WT-1 gene, the retinoblastoma gene (Lee, et al. (1987) Nature 329:642), the MMAC-1 gene, the adenomatous polyposis coli protein (U.S. Pat. No. 5,783,666), the deleted in colon carcinoma (DCC) gene, the MMSC-2 gene, the NF-1 gene, nasopharyngeal carcinoma tumor suppressor gene that maps at chromosome 3p21.3. (Cheng, et al. (1998) Proc. Nat. Acad. Sci. 95:3042-3047), the MTS1 gene, the CDK4 gene, the NF-1 gene, the NF2 gene, and the VHL gene.

The term “toxin gene” refers to nucleotide sequence, the expression of which in a cell produces a toxic effect. Examples of such toxin genes include nucleotide sequences encoding pseudomonas exotoxin, ricin toxin, diptheria toxin, and the like.

The term “pro-apoptotic gene” refers to a nucleotide sequence, the expression thereof results in the programmed cell death of the cell. Examples of pro-apoptotic genes include p53, adenovirus E3-11.6K, the adenovirus E4orf4 gene, p53 pathway genes, and genes encoding the caspases.

The term “pro-drug activating genes” refers to nucleotide sequences, the expression of which, results in the production of protein capable of converting a non-therapeutic compound into a therapeutic compound, which renders the cell susceptible to killing by external factors or causes a toxic condition in the cell.

The term “cytokine gene” refers to a nucleotide sequence, the expression of which in a cell produces a cytokine Examples of such cytokines include GM-CSF, the interleukins, especially IL-1, IL-2, IL-4, IL-12, IL-10, IL-19, IL-20, interferons of the alpha, beta and gamma subtypes especially interferon alpha-2b and fusions such as interferon alpha-2-alpha-1.

Modifications and/or deletions to the above referenced genes so as to encode functional subfragments of the wild type protein may be readily adapted for use in the practice of the present disclosure.

The disclosure further includes use of gene-targeted non-viral vectors. “Non-viral vector” for use in this aspect of the disclosure include autonomously replicating, extrachromosomal circular DNA molecules, distinct from the normal genome and nonessential for cell survival under non-selective conditions capable of effecting the expression of a DNA sequence in the target cell. Plasmids autonomously replicate in bacteria to facilitate bacterial production. Additional genes can be included to allow selection or screening for the presence of the recombinant vector.

In order to target the therapeutic gene to neoplastic, or damaged (e.g., pathogen-infected) cells, it is advantageous, in certain instances, to incorporate additional elements into non-viral gene delivery systems which facilitate cellular targeting. For example, a lipid encapsulated expression plasmid may incorporate SLC7A5 antibodies or ligands to facilitate targeting. Although a simple liposome formulation may be administered, the liposomes either filled or decorated with a desired composition of the disclosure of the disclosure can delivered systemically, or can be directed to a tissue of interest, where the liposomes then deliver the selected therapeutic/immunogenic peptide compositions. SLC7A5 antibodies and ligand for use in this application include antibodies, monoclonal antibodies, humanized antibodies, single chain antibodies, chimeric antibodies or functional fragments (Fv, Fab, Fab′) thereof. Alternatively, non-viral vectors can be linked through a polylysine moiety to a targeting moiety as described in Wu, et al. U.S. Pat. No. 5,166,320 and U.S. Pat. No. 5,635,383.

Therapies

The disclosure provides for treatment or prevention of cancer, including, but not limited to, neoplasms, tumors, metastases, or any disease or disorder characterized by uncontrolled cell growth by the administration of therapeutically or prophylactically effective amounts of anti-SLC7A5 antibodies or nucleic acid molecules encoding said antibodies. Examples of types of cancer and proliferative disorders to be treated with the SLC7A5-targeted therapeutics of the disclosure include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the disclosure are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). The treatment and/or prevention of cancer includes, but is not limited to, alleviating symptoms associated with cancer, the inhibition of the progression of cancer, the promotion of the regression of cancer, and the promotion of the immune response. In one embodiment, commercially available or naturally occurring anti-SLC7A5 antibodies, functionally active fragments or derivatives thereof are used in the present disclosure.

The SLC7A5 therapeutics may be administered alone or in combination with other types of cancer treatments (e.g., radiation therapy, chemotherapy, hormonal therapy, immunotherapy and anti-tumor agents). Examples of anti-tumor agents include, but are not limited to, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, and taxol. In one embodiment, one or more anti-SLC7A5 antibodies are administered to an animal, a mammal, or a human, after surgical resection of cancer. In another embodiment, one or more anti-SLC7A5 antibodies are administered to an animal, a mammal, or a human, in conjugation with chemotherapy or radiotherapy. In another embodiment, one or more anti-SLC7A5 antibodies are administered to an animal, a mammal, or a human, for the prevention or treatment of cancer prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of plasma to the animal.

The anti-SLC7A5 antibodies, and other SLC7A5-targeted therapeutics described herein, may be administered to an animal, mammal, or a human, for the prevention or treatment of cancer prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of IgG antibodies, IgM antibodies and/or one or more complement components to the animal. In another embodiment, one or more anti-SLC7A5 antibodies are administered to an animal, a mammal, or a human, prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of antibodies immunospecific for one or more cancer cell antigens. In yet another embodiment, one or more anti-SLC7A5 antibodies are administered to an animal, a mammal, or a human, for the prevention or treatment of cancer prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of antibodies currently used for the treatment of cancer. Examples of such antibodies include, but are not limited to, Herceptin, Retuxan, OvaRex, Panorex, BEC2, IMC-C225, Vitaxin, Campath I/H, Smart MI95, LymphoCide, Smart I D10, and Oncolym.

The disclosure further provides methods for the treatment or prevention of viral and other pathogen infections in an animal, a mammal, or a human, said methods comprising the administration of a therapeutically or prophylactically effective amount of anti-SLC7A5 antibodies or nucleic acid molecules encoding said antibodies or other SLC7A5-targeted therapeutics described herein. Examples of viral infections which can be treated or prevented in accordance with this disclosure include, but are limited to, viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). The treatment and/or prevention of a viral infection includes, but is not limited to, alleviating symptoms associated with said infection, the inhibition or suppression of viral replication, and the enhancement of the immune response.

The SLC7A5-targeted therapeutics described herein may be administered alone or in combination with other types of anti-viral or other anti-pathogen agents. Examples of anti-viral agents include, but are not limited to: cytokines (e.g., IFN-.alpha., IFN-.beta., and IFN-.gamma.); inhibitors of reverse transcriptase (e.g., AZT, 3TC, D4T, ddC, ddI, d4T, 3TC, adefovir, efavirenz, delavirdine, nevirapine, abacavir, and other dideoxynucleosides or dideoxyfluoronucleosides); inhibitors of viral mRNA capping, such as ribavirin; inhibitors of proteases such HIV protease inhibitors (e.g., amprenavir, indinavir, nelfinavir, ritonavir, and saquinavir); amphotericin B; castanospermine as an inhibitor of glycoprotein processing; inhibitors of neuraminidase such as influenza virus neuraminidase inhibitors (e.g., zanamivir and oseltamivir); topoisomerase I inhibitors (e.g., camptothecins and analogs thereof); amantadine and rimantadine. For example, one or more anti-SLC7A5 antibodies-drug conjugates are administered to an animal, a mammal, or a human, for the prevention or treatment of a viral infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of plasma to the animal.

In other examples, one or more SLC7A5-targeted therapeutics are administered to a patient for the prevention or treatment of a viral infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of IgG antibodies, IgM antibodies and/or one or more complement components to the animal. In another embodiment, anti-SLC7A5 antibodies are administered to an animal, mammal, or human, for the prevention or treatment of a viral infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of antibodies immunospecific for one or more viral antigens. Example of antibodies immunospecific for viral antigens include, but are not limited to, Synagis®, PRO542, Ostavir, and Protovir.

The disclosure further provides methods for the treatment or prevention of microbial infections in a patient, the said methods comprising the administration of a therapeutically or prophylactically effective amount of anti-SLC7A5-targeted therapeutics. Examples of microbial infections which can be treated or prevented in accordance with this disclosure include, but are not limited to, yeast infections, fungal infections, protozoan infections and bacterial infections. Bacteria which cause microbial infections include, but are not limited to, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophile, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, and Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori. The treatment and/or prevention of a microbial infection includes, but is not limited to, alleviating symptoms associated with said infection, the inhibition or suppression of replication, and the enhancement of the immune response.

SLC7A5-targeted therapeutics may be administered alone or in combination with other types of anti-microbial agents. Examples of anti-microbial agents include, but are not limited to: antibiotics such as penicillin, amoxicillin, ampicillin, carbenicillin, ticarcillin, piperacillin, cepalospolin, vancomycin, tetracycline, erythromycin, amphotericin B, nystatin, metroidazole, ketoconazole, and pentamidine. In one embodiment, a SLC7A5-targeted therapeutic is administered to an animal, a mammal, or a human, for the prevention or treatment of a microbial infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after) or concomitantly with the administration of plasma to the animal.

In certain instances, one or more SLC7A5-targeted therapeutics are administered to an animal, mammal, or human, for the prevention or treatment of a microbial infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of IgG antibodies, IgM antibodies and/or one or more complement components to the animal. In other instances, one or more SLC7A5-targeted therapeutics are administered to an animal, mammal, or human, for the prevention or treatment of a microbial infection prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the administration of antibodies immunospecific for one or more microbial antigens. Example of antibodies immunospecific for microbial antigens include, but are not limited to, antibodies immunospecific for LPS and capsular polysaccharide 5/8. In certain embodiments, animals with increased risk of a viral or bacterial infection are administered a composition of the disclosure. Examples of such animals include, but are not limited to, human burn patients, infants, immunocompromised or immunodeficient humans, and the elderly.

### 1.10 Kits

The disclosure further provides kits for use in diagnostics or prognostic, as well as therapeutic, methods for neoplasias and multidrug resistant neoplasias. The diagnostic kits are useful, for example, for detecting cell surface SLC7A5-expressing neoplasias in a patient sample or in situ in a patient. For example, during the course of patient chemotherapeutic treatment, monitoring of cell surface SLC7A5, and other markers described herein, provides valuable information regarding the efficacy. For example, the kit can comprise a labeled compound or agent capable of detecting cell surface SLC7A5 protein in a biological sample; as well as means for determining the amount of cell surface SLC7A5 in the sample; and means for comparing the amount of SLC7A5 in the sample with a standard (e.g., normal non-neoplastic cells). The compound or agent can be packaged in a suitable container. The kit can further comprise instructions for using the kit to detect cell surface SLC7A5 protein, as well as other markers. Such a kit can comprise, e.g., one or more antibodies capable of binding specifically to at least a portion of a cell surface SLC7A5 protein.

### 1.11 SLC7A5 Vaccines

Immunological compositions, including vaccines, and other pharmaceutical compositions containing the SLC7A5 protein, or portions thereof, are included within the scope of the present disclosure. One or more of the SLC7A5 proteins, or active or antigenic fragments thereof, or fusion proteins thereof can be formulated and packaged, alone or in combination with other antigens, using methods and materials known to those skilled in the art for vaccines. The immunological response may be used therapeutically or prophylactically and may provide antibody immunity or cellular immunity, such as that produced by T lymphocytes.

To enhance immunogenicity, the proteins may be conjugated to a carrier molecule. Suitable immunogenic carriers include proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular mass of at least 1,000 Daltons, or greater than 10,000 Daltons. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. An immune response is produced when the immunogen is injected into animals such as mice, rabbits, rats, goats, sheep, guinea pigs, chickens, and other animals, most mice and rabbits. Alternatively, a multiple antigenic peptide comprising multiple copies of the protein or polypeptide, or an antigenically or immunologically equivalent polypeptide may be sufficiently antigenic to improve immunogenicity without the use of a carrier.

The SLC7A5 protein or portions thereof, such as consensus or variable sequence amino acid motifs, or combination of proteins may be administered with an adjuvant in an amount effective to enhance the immunogenic response against the conjugate. One adjuvant widely used in humans has been alum (aluminum phosphate or aluminum hydroxide). Saponin and its purified component Quil A, Freund's complete adjuvant and other adjuvants used in research and veterinary applications are also available. Chemically defined preparations such as muramyl dipeptide, monophosphoryl lipid A, phospholipid conjugates such as those described by Goodman-Snitkoff et al. (1991) J. Immunol. 147:410-415 and incorporated by reference herein, encapsulation of the conjugate within a proteoliposome as described by Miller et al. (1992) J. Exp. Med. 176:1739-1744 and incorporated by reference herein, and encapsulation of the protein in lipid vesicles such as Novasome™ lipid vesicles (Micro Vescular Systems, Inc., Nashua, N.H.) may also be useful.

The disclosure includes the SLC7A5 polypeptide fragments, or subsequences of the intact SLC7A5 polypeptide shown in FIG. 12A (SEQ ID NO:1). Such SLC7A5 polypeptide subsequences, or a corresponding nucleic acid sequence that encodes them in the case of DNA vaccines, are selected so as to be highly immunogenic. The principles of antigenicity for the purpose of producing anti-SLC7A5 vaccines apply also to the use of SLC7A5 polypeptide sequences for use as immunogens for generating anti-SLC7A5 polyclonal and monoclonal antibodies for use in the SLC7A5-based diagnostics and therapeutics described herein.

Computer assisted algorithms for predicting polypeptide subsequence antigenicity are widely available. For example “Antigenic” looks for potential antigenic regions using the method of Kolaskar (see Kolaskar and Tongaonkar (1990) FEBS Letters 276:172-174 “A semi-empirical method for prediction of antigenic determinants on protein antigens”). In their initial study, Kolaskar and Tongaonkar experimentally tested 169 antigenic. The 156 which have less than 20 amino acids per determinant were selected (total 2066 residues). f(Ag) was calculated as the frequency of occurrence of each residue in antigenic determinants [f(Ag)=Epitope_occurrence/2066]. The Hydrophilicity, Accessibility and Flexibility values are from Parker, et al. (see Parker, et al. (1986) Biochemistry 25:5425-5432). In a given protein, the average for each 7-mer is calculated, and values are assigned to the central residue of the 7-mer. A residue is considered to be on the surface if any of the 7-mer values was above the average for the protein. These results were used to obtain f(s) as the frequency of occurrence of amino acids at the surface. The prediction algorithm includes the following steps: calculate the average propensity for each overlapping 7-mer and assign the result to the central residue (i+3) of the 7-mer; calculate the average for the whole protein; if the average for the whole protein is above 1.0 then all residues having above 1.0 are potentially antigenic; if the average for the whole protein is below 1.0 then all residues having above the average for the whole protein (note: the original paper has a mangled formula here) are potentially antigenic; find 6-mers where all residues are selected by step 3.

Another method for determining antigenicity of a polypeptide subsequence is the algorithm of Hopp and Woods ((1981) Proc. Natl. Acad. Sci. 86: 152-6). There are publicly available web sites for Hopp and Woods algorithm analysis of a user-input polypeptide sequence and convenient graphical output of the resulting analysis (see, e.g., http://hometown.aol.com/_ht_a/lucatoldo/myhomepage/JaMBW/3/1/7/). Using this algorithm to analyze the full-length human SLC7A5 sequence shown in FIG. 12A, several suitable sequence having a high Hopp and Woods antigenic index of an adequate length for immunogenicity were revealed. These include SLC7A5 amino acid residues: 45-60 (i.e., RPSTSRSLYASSPGGV); 295-315 (i.e., FADLSEAANRNNDALRQAKQE) and 330-345 (i.e., VDALKGTNESLERQMR).

In addition, the present disclosure provides a composition comprising the SLC7A5 protein or polypeptide fragment of the disclosure in combination with a suitable adjuvant. Such a composition can be in a pharmaceutically acceptable carrier, as described herein. As used herein, “adjuvant” or “suitable adjuvant” describes a substance capable of being combined with the SLC7A5 protein or polypeptide to enhance an immune response in a subject without deleterious effect on the subject. A suitable adjuvant can be, but is not limited to, for example, an immunostimulatory cytokine, SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. The adjuvant, such as an immunostimulatory cytokine can be administered before the administration of the SLC7A5 protein or SLC7A5-encoding nucleic acid, concurrent with the administration of the SLC7A5 protein or SLC7A5-encoding nucleic acid or up to five days after the administration of the SLC7A5 protein or SLC7A5-encoding nucleic acid to a subject. QS-21, similarly to alum, complete Freund's adjuvant, SAF, etc., can be administered within hours of administration of the fusion protein.

The disclosure may also utilize combinations of adjuvants, such as immunostimulatory cytokines co-administered to the subject before, after or concurrent with the administration of the SLC7A5 protein or SLC7A5-encoding nucleic acid. For example, combinations of adjuvants, such as immunostimulatory cytokines, can consist of two or more of immunostimulatory cytokines of this disclosure, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants may be determined by measuring the immune response directed against the SLC7A5 polypeptide with and without the adjuvant or combination of adjuvants, using standard procedures, as described herein.

Furthermore, the present disclosure provides a composition comprising the SLC7A5 protein or SLC7A5-encoding nucleic acid and an adjuvant, such as an immunostimulatory cytokine or a nucleic acid encoding an adjuvant, such as an immunostimulatory cytokine. Such a composition can be in a pharmaceutically acceptable carrier, as described herein. The immunostimulatory cytokine used in this disclosure can be, but is not limited to, GM/CSF, interleukin-2, interleukin-12, interferon-gamma, interleukin-4, tumor necrosis factor-alpha, interleukin-1, hematopoietic factor flt3L, CD40L, B7.1 con-stimulatory molecules and B7.2 co-stimulatory molecules.

The term “vaccine” as used herein includes DNA vaccines in which the nucleic acid molecule encoding SLC7A5 or antigenic portions thereof, such as any consensus or variable sequence amino acid motif, in a pharmaceutical composition is administered to a patient. For genetic immunization, suitable delivery methods known to those skilled in the art include direct injection of plasmid DNA into muscles (Wolff et al. (1992) Hum. Mol. Genet. 1:363), delivery of DNA complexed with specific protein carriers (Wu et al. (1989) J. Biol. Chem. 264:16985, coprecipitation of DNA with calcium phosphate (Benvenisty and Reshef (1986) Proc. Natl. Acad. Sci. 83:9551), encapsulation of DNA in liposomes (Kaneda et al. (1989) Science 243:375,), particle bombardment (Tang et al., (1992) Nature 356:152, and Eisenbraun et al. (1993) DNA Cell Biol. 12:791), and in vivo infection using cloned retroviral vectors (Seeger et al. (1984) Proc. Natl. Acad. Sci. 81:5849).

In another embodiment, the disclosure is a polynucleotide which comprises contiguous nucleic acid sequences capable of being expressed to produce a SLC7A5 or immunostimulant gene product upon introduction of said polynucleotide into eukaryotic tissues in vivo. The encoded gene product either acts as an immunostimulant or as an antigen capable of generating an immune response. Thus, the nucleic acid sequences in this embodiment encode an immunogenic epitope, and optionally a cytokine or a T-cell costimulatory element, such as a member of the B7 family of proteins.

Advantages to immunization with a gene rather than its gene product include the following. First, is the relative simplicity with which native or nearly native antigen can be presented to the immune system. Mammalian proteins expressed recombinantly in bacteria, yeast, or even mammalian cells often require extensive treatment to ensure appropriate antigenicity. A second advantage of DNA immunization is the potential for the immunogen to enter the MHC class I pathway and evoke a cytotoxic T cell response. Immunization of mice with DNA encoding the influenza A nucleoprotein (NP) elicited a CD8⁺ response to NP that protected mice against challenge with heterologous strains of flu. (Montgomery, D. L. et al. (1997) Cell Mol. Biol. 43(3):285-92; and Ulmer, J. et al. (1997) Vaccine 15(8):792-794). Cell-mediated immunity is important in controlling infection. Since DNA immunization can evoke both humoral and cell-mediated immune responses, its greatest advantage may be that it provides a relatively simple method to survey a large number of SLC7A5 genes and gene fragments for their vaccine potential.

The disclosure also includes known methods of preparing and using tumor antigen vaccines for use in treating or preventing cancers. For example, U.S. Pat. No. 6,562,347 which teaches the use of a fusion polypeptide including a chemokine and a tumor antigen which is administered as either a protein or nucleic acid vaccine to elicit an immune response effective in treating or preventing cancer. Chemokines are a group of usually small secreted proteins (7-15 kDa) induced by inflammatory stimuli and are involved in orchestrating the selective migration, diapedesis and activation of blood-born leukocytes that mediate the inflammatory response (see Wallack (1993) Annals New York Academy of Sciences 178). Chemokines mediate their function through interaction with specific cell surface receptor proteins. At least four chemokine subfamilies have been identified as defined by a cysteine signature motif, termed CC, CXC, C and CX₃ C, where C is a cysteine and X is any amino acid residue. Structural studies have revealed that at least both CXC and CC chemokines share very similar tertiary structure (monomer), but different quaternary structure (dimer). For the most part, conformational differences are localized to sections of loop or the N-terminus. In the instant disclosure, for example, a human SLC7A5 polypeptide sequence (such as that shown in FIG. 12A), or polypeptide fragment thereof, and a chemokine sequence are fused together and used in an immunizing vaccine. The chemokine portion of the fusion can be a human monocyte chemotactic protein-3, a human macrophage-derived chemokine or a human SDF-1 chemokine The SLC7A5 portion of the fusion is a portion shown in routine screening to have a strong antigenic potential.

### 1.12 Pharmaceutical Formulations and Methods of Treatment

The present disclosure provides for both prophylactic and therapeutic methods of treating a subject having a neoplastic disease. Subjects at risk for such a disease can be identified by a diagnostic or prognostic assay, e.g., as described herein. Administration of a prophylactic agent can occur prior to the manifestation of symptoms characteristic of the neoplasm, such that development of the neoplasm is prevented or, alternatively, delayed in its progression. In general, the prophylactic or therapeutic methods comprise administering to the subject an effective amount of a compound which comprises a SLC7A5 targeting component that is capable of binding to cell surface SLC7A5 present on neoplastic cells and which compound is linked to a therapeutic component.

Examples of SLC7A5 targeting components include monoclonal anti-SLC7A5 antibodies and fragments thereof. Examples of suitable therapeutic components include traditional chemotherapeutic agents such as Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, and Vinorelbine. Other examples of suitable therapeutic components include immunotoxins such as Pseudomonas exotoxin, a diphtheria toxin, a plant ricin toxin, a plant abrin toxin, a plant saporin toxin, a plant gelonin toxin, and pokeweed antiviral protein. Such immunotoxins are targeted to the SLC7A5 expressing neoplastic cell by the SLC7A5 targeting component of the therapeutic compound and, upon binding of cell surface SLC7A5 and uptake into the cell, function to kill or block the growth of the neoplastic cell.

### 1.12.1 Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic induces are useful. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

1.12.2 Formulation and Use

Pharmaceutical compositions for use in accordance with the present disclosure may be formulated in conventional manner using one or more physiologically acceptable carriers or excipients. Thus, the compounds and their physiologically acceptable salts and solvates may be formulated for administration by, for example, injection, inhalation or insulation (either through the mouth or the nose) or oral, buccal, parenteral or rectal administration.

For such therapy, the compounds of the disclosure can be formulated for a variety of loads of administration, including systemic and topical or localized administration. Techniques and formulations generally may be found in Remington's Pharmaceutical Sciences, Meade Publishing Co., Easton, Pa. For systemic administration, injection is useful, including intramuscular, intravenous, intraperitoneal, and subcutaneous. For injection, the compounds of the disclosure can be formulated in liquid solutions, in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present disclosure are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the disclosure are formulated into ointments, salves, gels, or creams as generally known in the art. A wash solution can be used locally to treat an injury or inflammation to accelerate healing.

In clinical settings, a therapeutic and gene delivery system for the SLC7A5-targeted therapeutic can be introduced into a patient by any of a number of methods, each of which is familiar in the art. For instance, a pharmaceutical preparation of the SLC7A5-targeted therapeutic can be introduced systemically, e.g., by intravenous injection.

The pharmaceutical preparation of the SLC7A5-targeted therapeutic compound of the disclosure can consist essentially of the compound in an acceptable diluent, or can comprise a slow release matrix in which the gene delivery vehicle or compound is imbedded.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Those skilled in the art will recognize, or be able to ascertain, using no more than routine experimentation, numerous equivalents to the specific substances and procedures described herein. Such equivalents are intended to be encompassed in the scope of the claims that follow the examples below.

### EXAMPLES

### Example 1

### Human Genome Oligoarray Experiments

### 1. Tumor Cell Lines

Human breast adenocarcinoma cell line MCF7, human ovarian adenocarcinoma cell line SKOV3, human ovarian carcinoma cell line 2008, human colorectal carcinoma cell lines T84 and HCT116, human lung carcinoma cell line A549, human non-small cell lung carcinoma cell line NC1-H460 and human prostatic adenocarcinoma cell line PC3 were obtained from ATCC (Manassas, Va., USA). All cell culture materials were obtained from Gibco Life Technologies (Burlington, Ont., Canada). The cell lines were cultured in αMEM medium supplemented with 10% fetal bovine serum (FBS) (MCF7) or with 15% FBS (SKOV3), in RPMI 1640 medium supplemented with 5% FBS (T84) or 10% FBS (H460) or 15% FBS (2008) or 20% FBS and 2.5% Glucose, 0.1 M HEPES, 10 mM MEM sodium pyruvate and 10 μmol/ml bovine insuline (OVCAR-3), in Dulbecco's Modified Eagle Medium supplemented with 10% fetal bovine serum (MDA), in McCoy's 5A Medium Modified supplemented with 10% FBS (HCT116), in HAM's F12 Medium supplemented with 10% FBS (A549, PC3). All culture media contained L-glutamine (final concentration of 2 mM). The cells were grown in the absence of antibiotics at 37° C. in a humid atmosphere of 5% CO₂ and 95% air. All cell lines were determined to be free of mycoplasma contamination using a PCR-based mycoplasma detection kit according to manufacturer's instructions commercially available (Stratagene Inc., San Diego, Calif., USA).

### 2. Cell RNA Extraction

Total RNA extraction from cell lines was done with RNEasy kit (Qiagen, USA), following the manufacturer's recommendations. Quantification of the RNA is done with the Nanodrop® ND-1000 spectrophotometer and the quality is assessed by the A₂₆₀/A₂₈₀ ratio. RNA preparations with an absorbance (A₂₆₀/A₂₈₀ ratio) of 1.9 to 2.3 were used for gene profiling experiments.

### 3. Normal Total RNA Groups

Total RNA groups for breast, ovarian, colon, lung and prostate were purchased from Biochain Institute Inc. (Hayward, USA). Standard clinical data were available for each patient included in the groups. Total RNA was extracted from snap frozen tissues samples using Trizol Reagent kit (Gibco-BRL, USA) extraction procedure. Total RNA was treated with RNA-free DNAse I and purified with the RNEasy kit (Qiagen, USA). RNA samples were visualized and analyzed on an Agilent 2100 BioAnalyzer (Agilent, USA) for purity and integrity.

### 4. Transcriptional Profiling

Fluorescently labeled cDNAs were prepared from 20 μg of total RNA for cancerous cell line and the normal human total RNA groups using the Agilent Fluorescent Direct Label kit (Agilent Technologies) using 1.0 mM cyanine 3- or 5-labeled dCTPs (Perkin Elmer, Waltham, Mass.) according to the manufacturer's instructions. cDNA preparations from tumor cell lines were cyanine-labeled and mixed with the reverse-color-labeled cDNA prepared from normal human total RNA group. Hybridizations were performed using the Agilent in situ Hybridization Plus kit according to the manufacturer's recommendations (Agilent Technologies). The combined cyanine 3- and 5-labeled cDNAs were denatured at 98° C. for 3 min, cooled to RT, and complemented with 50 μl of 10× control targets and 250 μl of 2× hybridization buffer. The labeled material was then applied to the Agilent Whole human genome oligo microarray (Agilent Technologies, #G4112A) consisting of 44,000 known and unknown human genes printed as 60-mer oligonucleotides using the SurePrint technology. The microarrays were hybridized in a hybridization rotation oven at 60° C. for 15 hr. The slides were disassembled in 6×SSC+0.005% Triton X-100, and washed with 6×SSC+0.005% Triton X-100 for 10 min at RT, followed by 5 min at 4° C. in 0.1×SSC+0.005% Triton X-100. Lastly, the slides were spun dry for 5 min at 1000 rpm. The microarrays were scanned with the ScanArray Lite scanner (Perkin Elmer), and the raw image data were extracted with the Packard BioScience QuantArray®

Microarray Analysis software. Data were analyzed with the ImaGene v6.0 software (BioDiscovery Inc, El Segundo, Calif.).

### 5. Microarray Data Analysis

The ImaGene® 6.0 was used to generate the lists of differentially expressed genes for each experiment. First, automated spot flagging analysis schemes were used to remove suspicious spots from any further analysis. Then, local methods for background correction measurement were applied. A log 10 transformation was done on the background-corrected data, followed by a global Lowess normalization step (based on intensity-dependent values) with a smoothing factor of 0.2. Finally, the background-corrected and normalized signals were analyzed to generate up and down regulated genes lists with a fold change threshold of 2.0. Moreover, a dye swap reaction was performed for one resistant/sensitive cell line (on the same day to account for potential differential incorporation of the labeled dCTPs used in the cDNA labeling reactions). Data analysis indicated that direct and reverse experiments performed with the same total RNA preparation gave similar gene profiling patterns, regardless of the date experiments were performed. When compared the greater than 10-fold up-regulated genes between the two experiments (direct and dye swap), 96% of them were the same. As for the down-regulated genes, 93% of them were the same in both experiments. Therefore, the tumor markers were selected based of the expression profiling done on the direct labeling experiment for the each of the cell lines tested.

Filtered- and Lowess-normalized ratios from the cancer cell line/normal human groups were analyzed to look for common differentially expressed genes in the different cell lines examined. Only the genes with a ratio of more than 5-fold increases (up-regulated in tumor versus normal group of the respective cancer) were considered for further analyses.

### 6. Selection of Tumor Biomarkers

In addition to the above analysis and the fold difference of up-regulated genes for each cancer, each of the up-regulated gene was selected only if the fold ratio was higher than 5 in the at least two tumor cell lines (e.g., for breast cancer, the two cell lines were MCF7 and MDA; for ovarian cancer, the three cell lines were SKOV3, 2008, and OVCAR-3; for colorectal cancer the two cell lines were T84 and HCT116; for lung cancer, the two cell lines were H460 and A549; for prostate cancer only one cell line was used, PC3 cells).

Five biomarkers, TTK, KIF20A, TRIM59, SLC7A5 and UHRF1, were selected to fit the selection criteria based on up-regulated genes in all the cancerous cell lines tested on the 44K Agilent oligoarray. These biomarkers are referred to as “PAN Cancer Biomarkers”, and are commonly up-regulated by at least 5-fold.

Table 2 shows the levels of SLC7A5 gene expression in cancer cell lines. For breast cancer, the two cell lines were MCF7 and MDA; for ovarian cancer, the three cell lines were SKOV3, 2008, and OVCAR-3; for colorectal cancer the two cell lines were T84 and HCT116; for lung cancer, the two cell lines were H460 and A549; for prostate cancer, PC3 cells were used.

Tumor cell lines were screened on the Agilent 44K 60-mer oligo microarray and SLC7A5 expression relative to normal groups was determines in relative fold of differential expression.

7. Validation of PAN Biomarkers mRNA Expression

Validation of the level of mRNA expression of the PAN biomarkers in the different cancers was done by relative quantification using quantitative Real-Time PCR. In brief, the delta-delta Ct method was used where the expression levels of the PAN biomarkers are quantified relative to the lung H23 adenocarcinoma cells, normalized to an exogenous reference gene (from Arabidopsis thaliana) and adjusted by taking into account the efficiencies of the PAN biomarkers and reference gene primers. Different aspects of the Real-Time PCR assay were optimized before the PAN Biomarkers mRNA levels in the different cancerous tissues were measured.

### 8. Quantitative Real-Time PCR Assay

The methodology used for the quantitative Real-Time PCR assay and that used for all the set-up and validation of the assay is as follows: Briefly, 500 ng of total RNA was mixed with 250 μg of pdN₆ random primers (GE Healthcare, Piscataway, N.J.), and 10 pg of Arabidopsis thaliana RNA, followed by 10 min incubation at 65° C. Samples were then cooled on ice for 2 min, and mixed with the cDNA synthesis solution to final concentrations of 50 mM Tris-HCl, pH 8.3, 75 mM KCL, 3 mM MgCL₂, 10 mM DTT, 1 nM dNTP (Roche Diagnostics, Canada), and 200 units of Superscript III RT enzyme (Invitrogen, USA). The samples were then incubated at 25° C. for 5 min, and 1 hr 30 min at 50° C. As a RT reaction control, 10 pg of RNA from Arabidopsis thaliana was added to each sample. When amplified by real-time PCR, the specific Arabidopsis thaliana gene is expressed at a known levels (Ct between 19 and 20), and therefore ensures that all RT reactions worked the same. That prevents the usage of a housekeeping gene to control for the amount of cDNA. For each sample, a No RT reaction was also performed, omitting the Superscript III enzyme. This ensures that no genomic DNA was present in the total RNA preparations. The optimal annealing temperature was 60° C. for SLC75. The Applied Biosystem taqman probes system (Foster City, USA) with the Light Cycler 480 (Roche Diagnostics, Canada) was used for this validation study. The reactions were prepared as followed: 10 μl Master Mix (final concentration of 1×), 1 μl taqman probe (final concentration of 1×), 4 μl of Rnase/Dnase-free water (Ambion, Canada), and 5 μl of cDNA or 5 μl of water (for No Template Control reactions) were added to each well for a final volume of 20 μl. As a reference sample, a calibrator of total RNA was prepared from the H23NSCLC adenocarcinoma cell line. This calibrator was used in each experiment, and the ratios to calibrator were calculated. This allowed for direct comparison between different experiments. In each test, duplicate wells were used for different controls to ensure that all reactions were reliable. Indeed, No Template Controls and No RT controls were included, an Arabidopsis thaliana gene was amplified, (as a normalization gene) and a calibrator sample was used to examine for consistency and accuracy.

The delta-delta Ct calculation method was used to analyze the real-time PCR data. Using this method, the cDNA synthesis and mRNA level are normalized with a calibrator (H23 total RNA). Briefly, the ddCt calculation compares the target gene Ct of each sample to the Ct of the calibrator for the same gene. This gives a ratio of expression relative to the calibrator (“referred to here as “the Normalized qPCR ratio”) and allows for comparison of the samples between experiments. The calibrator also accounts for the quality of the real-time experiment as it is always expressed at the same level in all genes tested. The mathematical equation for the relative quantification corrected for the efficiencies of the PAN biomarkers is as follows:

$$R = \frac{{( {E}_{target} )}^{Δ CPtarget ( control - sample )}}{{( {E}_{ref} )}^{Δ CPref ( control - sample )}}$$

### Example 2

### Quantitative Real-Time PCR Assays Setup

### 1. Preparation of the Total RNA Calibrator

To determine the exact levels of expression of each PAN biomarker by quantitative Real-Time PCR, a calibrator cell line was used to which biomarker expression levels for each gene in patient tissues is compared to under identical reaction conditions. The calibrator was used in each experiment and allowed the comparison of different experiments. A representative range of Ct values were sought that could allow the proper quantification of each biomarker expression levels in patient samples. Preliminary experiments were done with two lung cell lines, the H23 adenocarcinoma (NSCLC) and the HFL-1 embryonic lung fibroblast cell lines. The two lung cell lines were cultured from frozen stocks in the absence of antibiotics in F-12K Nutrient mix (HFL-1 cells) or modified RPMI media (H23 cells). RNA was extracted from cells collected at various passages using the commercial RNeasy Mini Kit (Qiagen, USA). Gene expression levels for each of the biomarkers were tested in a two-step qRT-PCR, Reverse transcription and qPCR reaction was conducted as described previously. Under the conditions tested, the two tumor cell lines showed a good range for gene expression levels. For the purpose of this work, the H23 adenocarcinoma cells were selected.

### 2. Verification of Probe Specificity and Primer Specificities

Real-Time PCR reaction products saved from the calibrator testing above were resolved on 2% agarose gels to verify the primers/probe specificity in both H23 and HFL-1 cell lines. A 60° C. PCR annealing temperature was optimal for KIF20A, SLC7A5, and UHRF1, however multiple bands were seen with TTK and TRIM59 primers. The latter multiple bands were resolved by increasing the annealing temperature to 62° C. and 64° C. which increase primer binding stringency for TTK and TRIM59 primers.

### 3. Assay Optimization

Following probe optimization, a small batch of H23 total RNA calibrator was prepared to verify the conditions of RNA extraction and DNAse treatment (i.e., the complete removal of genomic DNA (gDNA) from the RNA preparation). Three out of six reverse transcription reaction lacking the RT enzyme (no RT controls) gave a fluorescence signals. Moreover, DNA gel electrophoresis of the qRT-PCR products showed high molecular weight amplicons in the not RT controls, indicative of the persistence of gDNA. A 45 min DNAse digestion was done and DNA gel electrophoresis showed the disappearance of the high molecular weight amplicons. Using these latter optimized conditions, a large amount of total RNA was extracted from the H23 cells for cDNA calibrator preparation.

Using H23 cDNA preparation, standard curves of multiple replicates for each data point were set-up across a 10-fold serial dilution of the H23 cDNA (1:1 to 1:10,000). Using these standard curves, the amplification effiencies and optimal qPCR annealing temperatures for each of the five PAN biomarker primers, including those for the Arabidopsis thaliana reference gene, were optimized. The standard curves were used to calculate the normalized ratio of each patient and to generate primer efficiencies, which correct the equation for relative quantification. Roche LightCycler 480 software was used to generate plots of Ct versus log of the dilution, and the slope of the line was used to calculate primer efficiencies using the equation E=10-1/slope-1. Five taqman probe/primers sets had acceptable efficiencies of between 1.78 and 2.2, and errors of less than 0.2.

### 4. Optimization of Patient Total RNA Required for Real-Time PCR

To determine the optimal quantity of patient RNA to be tested (i.e., the amount that will give Ct values that lie within the standard curves), RNA samples from one NSCLC patient and one normal lung individual were quantified by Nanoprop to obtain 100 ng, 250 ng, and 500 ng of total RNA. Separate reverse transcription reactions were set-up as described above for each of these three quantities of RNA for both patient samples, and qPCR was performed on the six samples using the five optimized primer/probes combinations. Expression levels from the six samples were inspected to determine which of the three starting total RNA amounts (in nanograms) are within range of the Cts covered by each PAN biomarker standard curve. 500 ng is the optimal quantity of patient RNA for reverse-transcription qPCR in order to obtain Ct values that could be accurately quantified by standard curves without having to extrapolate.

### Example 3

### Validation of the PAN Biomarkers in Clinical Samples

Five different groups of patients were studied: The lung cancer group consisted of non-small cell lung cancer (NSCLC) patients with a variety of subtypes (mainly adenocarcinomas and squamous cell carcinomas. Patients within the lung cancer group had an average age of 62.5 years and were mostly male. Early disease stages were well represented (I-II) (with only one stage III patient) in this group samples. The Breast Cancer Group was of an average age of 53.1 years with a majority of Caucasian women. Stages I and II breast cancer are equally represented in this group, as well as the women menopausal status. For the breast cancer patients, the majority of the cases were infiltrating ductal carcinoma. The Ovarian Cancer Group of patients was of an average age of 61.5 and patients diagnosed with serous adenocarcinomas stage III, mostly menopausal. The Colorectal Cancer Group, patients were only males with an average age of 69.7 years. Cases were distributed equally between stages Ito III and were classified as adenocarcinoma of the colon. The Prostate Cancer Group, patients were of an average age of 62 years with stage II prostate cancer. The majority of patients were diagnosed with adenocarcinoma of the prostate.

The normal patients for each cancer were coming from different individuals (lung, breast and ovary) except for colon and prostate cases. For the latter two cancers, the normal samples were normal matched samples from the same patients.

For breast, ovarian and lung patients, total RNA samples were obtained from several tissue diposatories [Asterand Inc. (Detroit, USA), Clinomics Biosciences Inc (Watervliet, USA) and Biochain Institute Inc. (Hayward, USA). Total RNA was extracted from snap frozen tissues samples using Trizol Reagent kit (Gibco-BRL, USA) extraction procedure. Total RNA was treated with RNA-free DNAse I and purified with the RNEasy kit (Qiagen, USA). RNA samples were visualized and analyzed on an Agilent 2100 BioAnalyzer (Agilent, USA) for purity and integrity.

For the colorectal and prostate cancers, patients samples were obtained from Indivumed Inc (Hamburg, Germany) as 10 μm formalin-fixed paraffin embedded (FFPE) sections. Total RNA was extracted from FFPE section using the High pure RNA paraffin kit (Roche) with some modifications. Briefly, the paraffin sections were deparaffinized by incubation in Citrosolv (Fisher) for 10 min and washed 2× with 99% ethanol for 10 min. After the final wash, the paraffin sections were scratch and the material was air-dried at 55° C. for 10 min. Each sample was incubated with 100 μm Tissue Lysis Buffer, 16 μl 10% SDS and 40 μl proteinase K, homogenized and incubated overnight at 55° C. After proteinase K digestion, RNA was isolated by the addition of 325 μl Binding Buffer and 325 μl ETOH 99% and gently mixed. The lysate was added to the column and centrifuged at 8,000 rpm for 30 sec, at RT. The sample was dried completely by centrifugation at 12,000 rpm for 30 sec, and washed with 500 μl Wash Buffer I, followed by two washed with Wash buffer II. After each wash, the sample was centrifuged at 8,000 g for 20 sec and the flow through was discarded. A last centrifugation was done at 12 000 rpm for 2 min to ensure that the entire buffer was removed. RNA was eluted with 90 μl of elution buffer, by incubation for one min at RT, and a centrifugation at 8,000 g for 1 min. To remove genomic DNA, all samples were incubated with 2 μl of DNase 5 U/μl (Roche) at 37° C. for 1 hr. After the DNase treatment, the sample were homogenized and incubated in digestion buffer with proteinase K (20 μl Tissue Lysis Buffer, 10% SDS 40 μl, Proteinase K) at 55° C. for 1 hr. RNA was isolated, washed and collected by centrifugation after incubation at RT for 1 min with 50 μl of elution buffer. Lastly, the amount of RNA in the samples was measured using the Nanodrop® ND-1000 spectrophotometer. The purity of the RNA extracted from each FFPE tissue samples was evaluated by the 260/280 ratio obtained during the RNA quantification (Nanodrop® ND-100 spectrophotometer).

### Example 4

### Receiver Operating Characteristic (ROC) Curves

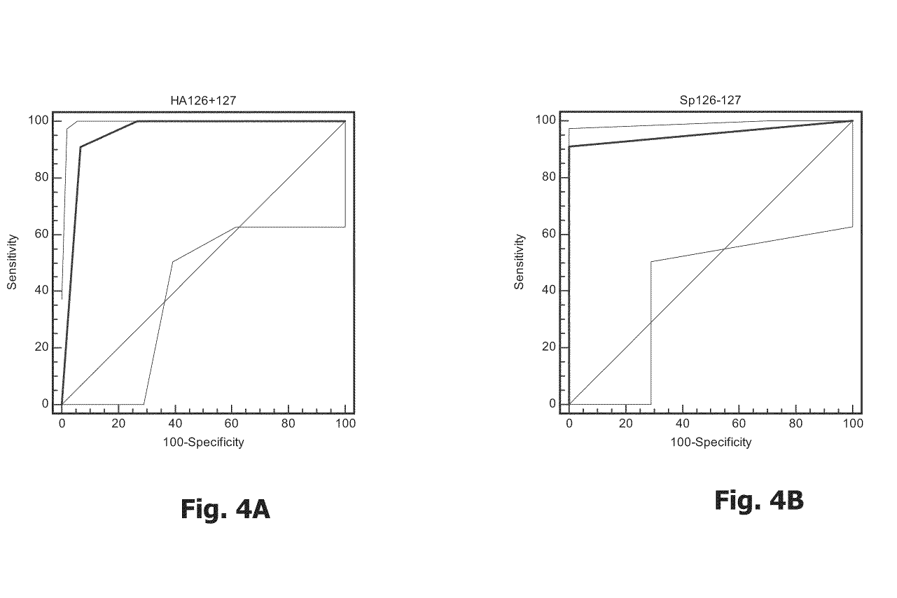

Receiver operating curves were done with the MedCal software using the normalized qPCR ratios obtained during the qRT-PCR analyses of each PAN biomarkers on the panel of cancerous patients tested. Each cancer was analyzed separately. ROC curves were generated for each biomarker and area under the curve (AUC), sensitivity and specificity were obtained. Further analyses were done using the cut-off value obtained under the high accuracy setting and using the cut-off value calculated by the software when the specificity of the assay is set to 100% (no false positive result). Combinations of PAN biomarkers were assessed using a scoring system based on the cut-off values (high accuracy and 100% specificity). In summary, for each patient, a score of 1 was given when the ratio obtained for the biomarker was superior to the cut-off value of that biomarker. Then, for each patient, a sum of the score obtained for each target was compiled and used for the ROC curve analysis. The results are shown in FIGS. 3 and 4 (lung), 7 and 8 (breast), 10 and 11 (ovarian), and 13-14 (colorectal).

### Example 5

### Real-Time Quantitive PCR for the Detection SLC7A5 in Samples Obtained From Normal Lung Subjects and Lung Cancer Patients

1. Total RNA Isolation and cDNA Labeling

Patient tissues samples were obtained from Asterand, Inc. (Detroit, Mich.), and Biochain Institute, Inc. (Hayward, Calif.). Each patient included in the study was screened against the same normal total RNA group in order to compare them together. The tumor group was composed of 11 cases. The lung normal group was composed of 15 cases.

### 2. Real-Time PCR

Real-time PCR and analysis of results are performed as shown in Example 2. ROC curves were prepared as described in Example 4.

### 3. ROC Analysis

To determine the predictive values of measuring the differential expression of SLC7A5, alone and in combination with TTK, KIF20A, TRIM59 and/or UHRF1 for lung cancer, the expression levels of these PAN biomarkers RNAs were analyzed using ROC curves.

### 4. Results

Increased levels of SLC7A5 mRNA were detected in tumor fluid and cell samples obtained patients suffering from non-small lung cancer compared to the levels in fluid and cell samples obtained from normal lung subjects (FIG. 1). Tumor samples from patients suffering from lung cancer averaged about 25.3 times higher levels of SLC7A5 mRNA expression than found in normal subjects. (Table 1). These results establish that SLC7A5 is a marker of neoplastic disease in lung.

Similarly, the differential expression of TTK, KIF20A, TRIM59 and UHRF1 mRNAs was measured in the same NSCLC patients using quantitative Real-Time PCR technique. NSCLC tumors showed a much higher levels of RNA expression for KIF20A. In comparison to the other cancers tested, the fold increase measured in the lung cancer are high for all five PAN biomarkers and may reflect the results seen with the whole human genome studies in cancerous cell lines.

ROC curves analyses were done for each PAN biomarker separately and in combination. For NSCLC samples, a good area under the curve (AUC) was obtained for SLC7A5 (FIGS. 3-4) and for each of the other four PAN biomarkers. With the high accuracy cut-off value, sensitivity and specificity was obtained for all the PAN biomarkers. However, when the cut-off values selected are the ones that give 100% specificity, the sensitivity decreased to 72.7 to 81.8%. Perfect AUC (100%) is obtained when all the PAN biomarkers are combined at high accuracy (at least two biomarkers is over their cut-off values) but decrease to 96% when there is 100% specificity (sensitivity of 90.9%). In that case, the score need to be of at least one, meaning that only one biomarker needs to have a normalized qPCR ratio over its cut-off value, as shown in Table 4.

TABLE 4

| | | High Accuracy | | | | 100% Specificity | | |
|---|---|---|---|---|---|---|---|---|
| Target | Auc | Sensitivity | Specificity | Cut-off | Auc | Sensitivity | Specificity | Cut-off |
| KIF20A | 0.94 | 88.2 | 90.9 | >0.21 | | 52.9 | 100 | >0..46 |
| SLC7A5 | 0.84 | 76.5 | 81.8 | >0..03 | | 29.4 | 100 | >0.13 |
| TRIM59 | 0.98 | 94.1 | 100.0 | >0.7 | | 94.1 | 100 | >0.7 |
| TTK | 0.995 | 94.1 | 100 | >0.1 | | 94.1 | 100 | >0.1 |
| UHRF1 | 0.85 | 100 | 72.7 | >0.009 | | 29.4 | 100 | >0.12 |
| KIF20A + SLC7A5 | 0.90 | 94.1 | 81.8 | >score 0 | 0.77 | 52.9 | 100 | >score 0 |
| KIF20A + TRIM59 | 0.97 | 88.2 | 100.0 | >score 1 | 0.97 | 94.1 | 100 | >score 0 |
| KIF20A + TTK | 0.97 | 88.2 | 100 | >score 1 | 0.97 | 94.1 | 100 | >score 0 |
| ABp125 + 129 | 0.84 | 88.2 | 82 | >score 0 | 0.79 | 58.8 | 100 | >score 0 |
| SLC7A5 + TRIM59 | 0.97 | 100.0 | 81.8 | >score 0 | 0.97 | 94.1 | 100 | >score 0 |
| SLC7A5 + TTK | 0.97 | 100 | 82 | >score 0 | 0.97 | 94 | 100 | >score 0 |
| SLC7A5 + UHRF1 | 0.79 | 88.2 | 72.7 | >score 0 | 0.91 | 81.8 | 100 | >score 0 |
| TRIM59 + TTK | 0.91 | 94.1 | 81.8 | >score 0 | 0.77 | 52.9 | 100 | >score 0 |
| TRIM59 + UHRF1 | 0.91 | 94 | 81.8 | >score 0 | 0.97 | 94.1 | 100 | >score 0 |
| TTK + UHRF1 | 0.91 | 94.1 | 82 | >score 0 | 0.97 | 94.1 | 100 | >score 0 |
| PAN (5) | 0.98 | 94 | 91 | >score 1 | 0.97 | 94.1 | 100 | >score 0 |
| Potentially Secreted (2) | 0.97 | 100 | 82 | >score 0 | 0.97 | 94.1 | 100 | >score 0 |

### Example 6

### Western Blot Analysis of Samples Isolated from Lung Cancer Patients and Normal Lung Subjects

### 1. Patient Samples and Normal Samples

Patient lung tissues and pleural fluid samples are obtained from Asterand, Inc. (Detroit, Mich.), and Biochain Institute, Inc. (Hayward, Calif.). Each patient included in the study is screened against the same normal total RNA group in order to compare them together.

### 2. Western Blot Analysis of SLC7A5 in Lung Cancer and Normal Lung Samples

Fluid samples are prepared by in one of two ways: a) mixing total unfractionated pleural fluid with lysis buffer as described below; or b) the pleural fluid is first fractionated by centrifugation where both the pellet and supernatant material are mixed with lysis buffer. Protein lysates from a) and b) are then quantified and equal amounts of protein are resolved on SDS-PAGE and Western blotting.

For lung cell samples, human tissues are homogenized using a Polytron PT10-35 (Brinkmann, Mississauga, Canada) for 30 sec at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada).

40 μg of proteins from human lung tissue samples and fluid samples isolated from cancer patients and normal lung subjects are used in SDS-PAGE gels. Samples are mixed with Laemmli buffer, heated for 5 min at 95° C., and then resolved by 12% SDS-PAGE. Proteins are then electro-transferred onto Hybond-ECL nitrocellulose membranes (Amersham Biosciences, Baie d'Urfé, Canada) for 90 min at 100 volts at RT. Membranes are blocked for 1 hr at RT in blocking solution (PBS containing 5% fat-free dry milk). Membranes are washed with PBS and are incubated with the primary anti-SLC7A5 antibodies at the appropriate dilutions in blocking solution containing 0.02% sodium azide for 2 hr at RT. PBS washing is performed, and the membranes are subsequently incubated for 1 hr at RT with secondary anti-mouse, anti-rabbit or anti-goat antibodies labeled with horseradish peroxydase (Bio-Rad, Mississauga, Canada) diluted 1/3000 in PBS. Chemiluminescence detection is performed using the SuperSignal West Pico Chemiluminescent Substrate (Pierce, Rockford, Ill., USA) following the manufacturer's recommendations.

### 3. Results

SLC7A5 expression is significantly increased in cell and fluid samples obtained from lung tumor patients as compared to expression in cell and fluid samples isolated from normal subjects. All normal subjects show nearly undetectable levels of SLC7A5 protein expression, while samples obtained from lung cancer patients show detectable levels of SLC7A5.

### Example 7

### Real-Time Quantitive PCR for the Detection of SLC7A5 in Samples Obtained From Normal Breast Subjects and Breast Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA group in order to compare them together. The tumor group was composed of 17 cases. The breast normal group was composed of 10 cases.

### 2. Real-Time PCR

Real-time PCR and analysis of results are performed as shown in Example 2. ROC curves were prepared as described in Example 4.

### 3. ROC Analysis

To determine the predictive values of measuring the differential expression of SLC7A5, alone and in combination with TTK, KIF20A, TRIM59 and/or UHRF1 for breast cancer, the expression levels of these PAN biomarkers RNAs were analyzed using ROC curves.

### 4. Results

Increased levels of SLC7A5 mRNA were detected in tumor fluid and cell samples obtained patients suffering from breast cancer compared to the levels in fluid and cell samples obtained from normal breast subjects (FIGS. 5-6). Tumor samples from patients suffering from breast cancer averaged about 13.4-fold higher levels of SLC7A5 mRNA expression than found in normal subjects (Table 1). These results establish that SLC7A5 is a marker of neoplastic disease in breast.

Similarly, the differential expression of the four biomarkers, e.g., TTK, KIF20A, TRIM59 and UHRF1, mRNAs was measured in the same breast patients using quantitative Real-Time PCR technique. The results show significant differences in RNA expression for each of the PAN biomarkers between the breast samples and normal breast samples from patients.

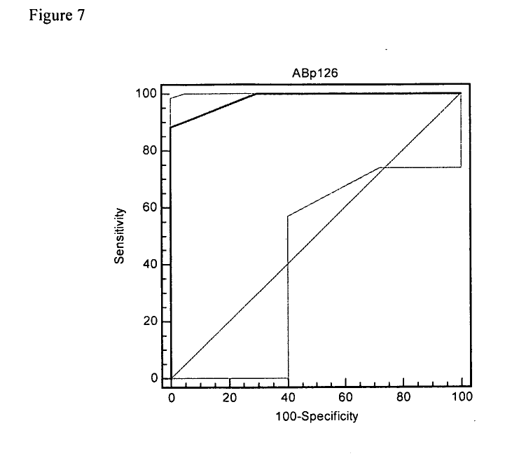

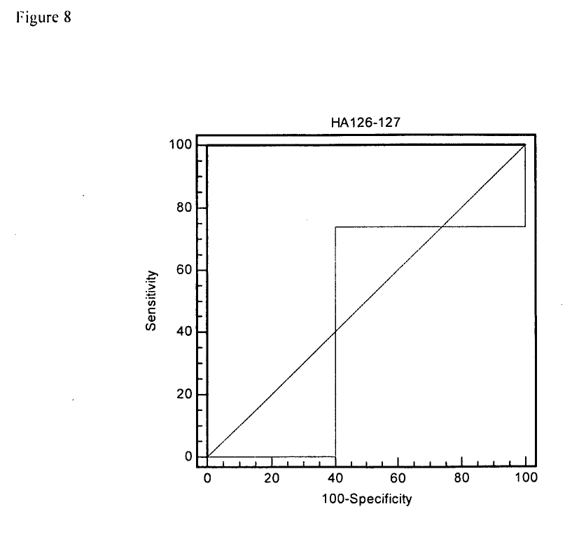

ROC curves analyses were done for each PAN biomarker separately (FIG. 7 for SLC7A5) and in combination. The results in Table 5 summarize the performances of SLC7A5 and the other biomarkers in breast cancer samples.

TABLE 5

| | | High Accuracy and | | |
|---|---|---|---|---|
| | | 100% Specificity | | |
| Target | Auc | Sensitivity | Specificity | Cut-off |
| KIF20A | 0.991 | 94.12 | 100 | >0.02 |
| SLC7A5 | 0.982 | 88.24 | 100 | >0.02 |
| TRIM59 | 1 | 100 | 100 | >0.13 |
| TTK | 0.994 | 94.12 | 100 | >0.03 |
| UHRF1 | 1 | 100 | 100 | >0.01 |
| KIF20A + SLC7A5 | 1 | 100 | 100 | >score 0 |
| KIF20A + TRIM59 | 1 | 100 | 100 | >score 0 |
| KIF20A + TTK | 0.97 | 94.1 | 100 | >score 0 |
| KIF20A + UHRF1 | 1 | 100 | 100 | >score 0 |
| SLC7A5 + TRIM59 | 1 | 100 | 100 | >score 0 |
| SLC7A5 + TTK | 1 | 100 | 100 | >score 0 |
| SLC7A5 + UHRF1 | 1 | 100 | 100 | >score 0 |
| TRIM59 + TTK | 1 | 100 | 100 | >score 0 |
| TRIM59 + UHRF1 | 1 | 100 | 100 | >score 0 |
| TTK + UHRF1 | 1 | 100 | 100 | >score 0 |
| PAN (5) | 1 | 100 | 100 | >score 0 |
| Potentially secreted (2) | 1 | 100 | 100 | >score 0 |

### Example 8

### Western Blot Analysis of Samples Isolated From Breast Cancer Patients and Normal Breast Subjects

### 1. Patient Samples and Normal Samples

Patient breast tissues and pleural fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Western Blot Analysis of SLC7A5 in Breast Cancer and Breast Normal Samples

Fluid samples are prepared by in one of two ways: a) mixing total unfractionated pleural fluid with lysis buffer as described below; or b) the pleural fluid is first fractionated by centrifugation where both the pellet and supernatant material are mixed with lysis buffer. Protein lysates from a) and b) are then quantified and equal amounts of protein are resolved on SDS-PAGE and Western blotting.

For breast cell samples, human tissues are homogenized using a Polytron PT10-35 (Brinkmann, Mississauga, Canada) for 30 sec at speed setting of 4 in the presence of 300 μA of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada).

40 μg of proteins from human breast tissue samples and fluid samples isolated from cancer patients and normal breast subjects are used in SDS-PAGE gels. Samples are mixed with Laemmli buffer, heated for 5 min at 95° C., and then resolved by 12% SDS-PAGE. Proteins are then electro-transferred onto Hybond-ECL nitrocellulose membranes (Amersham Biosciences, Baie d'Urfé, Canada) for 90 min at 100 volts at RT. Membranes are blocked for 1 hr at RT in blocking solution (PBS containing 5% fat-free dry milk). Membranes are washed with PBS and are incubated with the primary anti-SLC7A5 antibodies at the appropriate dilutions in blocking solution containing 0.02% sodium azide for 2 hr at RT. PBS washing is performed, and the membranes are subsequently incubated for 1 hr at RT with secondary anti-mouse, anti-rabbit or anti-goat antibodies labeled with horseradish peroxydase (Bio-Rad, Mississauga, Canada) diluted 1/3000 in PBS. Chemiluminescence detection is performed using the SuperSignal West Pico Chemiluminescent Substrate (Pierce, Rockford, Ill., USA) following the manufacturer's recommendations.

### 3. Results

SLC7A5 expression is significantly increased in cell and fluid samples obtained from breast tumor patients as compared to expression in cell and fluid samples isolated from normal subjects. All normal subjects show nearly undetectable levels of SLC7A5 protein expression, while samples obtained from breast cancer patients show detectable levels of SLC7A5.

### Example 9

### ELISA Analysis of SLC7A5 in Breast Cancer and Breast Normal Tissues

### 1. Isolation and Preparation of Patient and Normal Tissues

Patient tissue samples were obtained and prepared as described in Example 3.

### 2. ELISA Analysis

To quantify the amount of each target of interest and to confirm the results obtained by Western blot, an ELISA technique was performed on ovarian samples for SLC7A5. Prior to screening all samples, an optimization of the conditions was performed using normal and tumor samples to determined the linearity of the assay (dose-dependant curve, time of development of the assay). Once conditions were optimized (Results to come), 96-well plates ((Maxisorp plates, NUNC, (Rochester, N.Y., USA)) were coated with the capture antibody. Samples were then incubated overnight at 4° C. Wells were washed 3 times with PBS and then blocked with bovine serum albumin (BSA)/PBS or BSA alone for 1 hr at RT. Detection antibodies (40 ng/well) were added to the wells and incubated for 2 hr RT. Plates were washed 3 times with PBS and the secondary anti-mouse, anti-rabbit or anti-goat antibodies labeled with horseradish peroxidase (Bio-Rad, Mississauga, Canada), diluted 1:3000 in 3% BSA/PBS, was incubated for 1 hr at RT. Wells were washed 3 times with PBS and developed with 2,2′-azino-bis(3-ethylbenzthiazoline-6-sulfonic acid) (ABTS) as the substrate (Sigma Corp., St. Louis, Mo.).

The intensity of the signal was assessed by reading the plates at A₄₀₅ nm wavelength using a microplate reader. For each of the target, a standard curve was established with a recombinant or purified protein at the same time to quantify the target in each sample. Results were expressed as concentrations of a target in 1 μg of total protein extract. All samples were quantified in the same assay. Differences among normal and tumor groups were analyzed using Student's two-tailed t test with significance level defined as P<0.05.

### 3. Results

ELISA results show the levels of SLC7A5 protein expression in normal and breast tissue samples. Results are shown as ng/μg of protein marker in each normal subject versus ng/μg of protein marker in each breast cancer patient. These results confirm the results obtained in the Western blot protein analysis.

### Example 10

### Real-Time Quantitative PCR for the Detection of SLC7A5 in Samples Obtained From Normal Ovarian Subjects and Ovarian Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA group in order to compare them together. The tumor group was composed of 17 cases. The ovarian normal group was composed of 10 cases.

### 2. Results

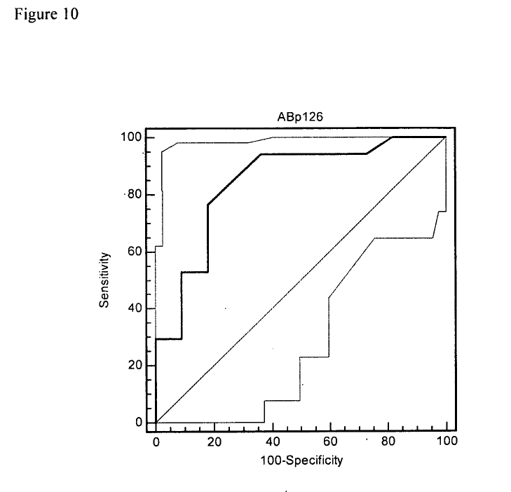

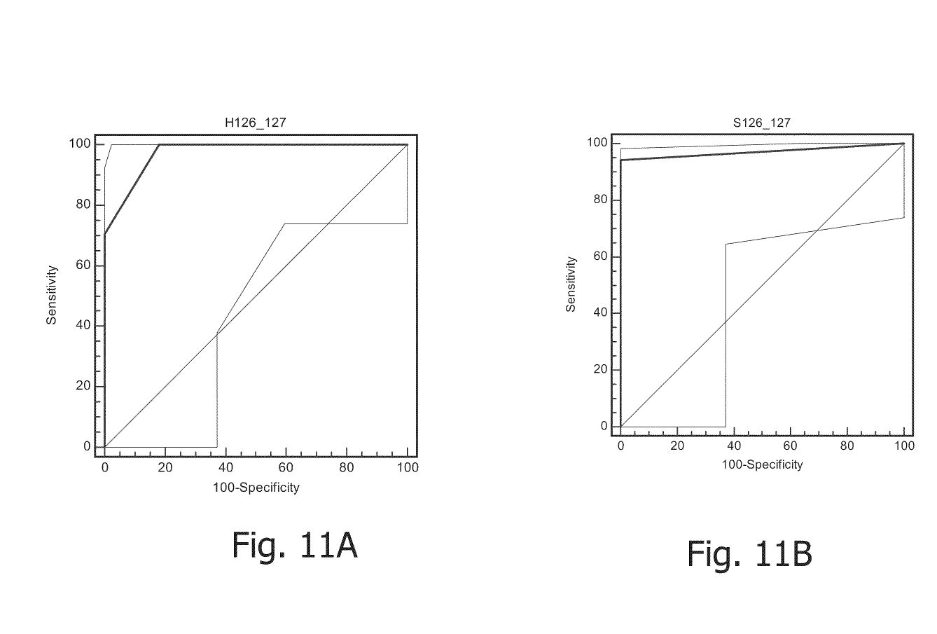

Increased levels of SLC7A5 mRNA were detected in tumor fluid and cell samples obtained patients suffering from ovarian cancer compared to the levels in fluid and cell samples obtained from normal ovarian subjects (FIG. 10). Tumor samples from patients suffering from ovarian cancer averaged about 4.2-fold higher levels of SLC7A5 mRNA expression than found in normal subjects (Table 1). These results establish that SLC7A5 is a marker of neoplastic disease in ovarian.

Similarly, the differential expression of the four biomarkers, e.g., TTK, KIF20A, TRIM59 and UHRF1, mRNAs was measured in the same ovarian patients using quantitative Real-Time PCR technique. There is a significant differences in RNA expression for each of the PAN biomarkers between the ovarian test samples and normal ovarian samples from patients.

To determine the predictive values of measuring the differential expression of SLC7A5 (FIG. 10), alone and in combination with TTK, KIF20A, TRIM59, and/or UHRF1 for breast cancer, the expression levels of these PAN biomarkers RNAs were analyzed using ROC curves. ROC curves analyses were done for each PAN biomarker separately and in combination (Table 6).

TABLE 6

| | | High Accuracy | | | | 100% Specificity | | |
|---|---|---|---|---|---|---|---|---|
| Target | Auc | Sensitivity | Specificity | Cut-off | Auc | Sensitivity | Specificity | Cut-off |
| KIF20A | 0.94 | 80.0 | 100.0 | >0.0036 | | 80.0 | 100 | >0.0036 |
| SLC7A5 | 1.00 | 100.0 | 100 | >0.0013 | | 100.0 | 100 | >0.0013 |
| TRIM59 | 0.90 | 80 | 90 | >0.0044 | | 60.0 | 100.0 | >0.0061 |
| TTK | 0.87 | 100.0 | 60.0 | >0.0022 | | 50.0 | 100.0 | >0.01 |
| UHRF1 | 0.96 | 90.0 | 100.0 | >0.0041 | | 90.00 | 100.00 | >0.0041 |
| KIF20A + SLC7A5 | 1 | 100.0 | 100.0 | >score 0 | 1.00 | 100 | 100 | >score 0 |
| KIF20A + TRIM59 | 0.90 | 80.0 | 100.0 | >score 0 | 0.90 | 80.0 | 100 | >score 0 |
| KIF20A + TTK | 0.95 | 80.0 | 100.0 | >score 1 | 0.90 | 80.0 | 100 | >score 0 |
| KIF20A + UHRF1 | 0.94 | 90.0 | 90.0 | >score 0 | 0.95 | 90 | 100 | >score 0 |
| SLC7A5 + TRIM59 | 1.00 | 100.0 | 100.0 | >score 0 | 1 | 100 | 100 | >score 0 |
| SLC7A5 + TTK | 1.00 | 100.0 | 100.0 | >score 1 | 1 | 100 | 100 | >score 0 |
| SLC7A5 + UHRF1 | 0.995 | 100.0 | 90.0 | >score 0 | 1 | 100 | 100 | >score 0 |
| TRIM59 + TTK | 0.90 | 60.0 | 100.0 | >score 1 | 0.80 | 60.0 | 100.0 | >score 0 |
| TRIM59 + UHRF1 | 0.93 | 90.0 | 90.0 | >score 0 | 0.95 | 90.0 | 100.0 | >score 0 |
| TTK + UHRF1 | 0.93 | 90.0 | 90.0 | >score 1 | 0.95 | 90.0 | 100.0 | >score 0 |
| PAN (5) | 0.995 | 100.00 | 90.0 | >score 1 | 1.00 | 100.0 | 100.0 | >score 0 |
| Potentially Secreted (2) | 1.00 | 100.0 | 100.0 | >score 0 | 1.00 | 100.0 | 90.0 | >score 0 |

### Example 11

### Western Blot Analysis of Samples Isolated from Ovarian Cancer Patients and Normal Ovarian Subjects

### 1. Patient Samples and Normal Samples

Patient tissue samples were obtained from Asterand, Inc. (Detroit, Mich.), Clinomics Biosciences, Inc (Watervliet, N.Y.) and Biochain Institute, Inc. (Hayward, Calif.). The samples were isolated from normal ovaries and ovarian cancer tissues, and were frozen into blocks of tissue. Protein cell extracts were then prepared from each block. Each patient included in the study was screened against the same normal total RNA group in order to compare them together. The tumor group composed of 36 cases. The ovarian normal group was composed of 34 cases.

### 2. Western Blot Analysis of SLC7A5 in Ovarian Cancer and Normal Ovarian Samples

For ovarian cell samples, human tissues were homogenized using a Polytron PT10-35 (Brinkmann, Mississauga, Canada) for 30 sec at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). 40 μg of proteins from human ovarian cancer patients and normal ovarian subjects were used in SDS-PAGE gels. Samples were mixed with Laemmli buffer (250 mM Tris-HCl, pH 8.0, 25% (v/v) b-mercaptoethanol, 50% (v/v) glycerol, 10% (w/v) SDS, 0.005% (w/v) bromophenol blue), heated for 5 min at 95° C. and resolved in 12% SDS-polyacrylamide gels (SDS-PAGE). Proteins were then electro-transferred onto Hybond-ECL nitrocellulose membranes (Amersham Biosciences, Baie d'Urfé, Canada) for 90 min at 100 volts at RT. Membranes were blocked for 1 hr at RT in blocking solution (PBS containing 5% fat-free dry milk). Membranes were washed with PBS and incubated with the primary anti-SLC7A5 polyclonal antibodies or monoclonal antibodies at the appropriate dilutions in blocking solution containing 0.02% sodium azide for 2 hr at RT. Antibodies were produced in house. PBS washing was performed, and the membranes were subsequently incubated for 1 hr at RT with secondary anti-mouse, anti-rabbit or anti-goat antibodies labeled with horseradish peroxydase (Bio-Rad, Mississauga, Canada) diluted 1/3000 in PBS. Chemiluminescence detection was performed using the SuperSignal West Pico Chemiluminescent Substrate (Pierce, Rockford, Ill., USA) following the manufacturer's recommendations.

### 3. Results

SLC7A5 expression was significantly increased in tumor samples obtained from ovarian tumor patients as compared to expression in samples from normal subjects. All normal subjects showed nearly undetectable levels of SLC7A5 protein expression, while nearly 60% of samples obtained from ovarian cancer patients showed detectable levels of SLC7A5.

### Example 12

### Real-Time Quantitative PCR for the Detection of SLC7A5 in Samples from Colon Cancer Patients and Normal Colon Subjects

### 1. Patient Samples and RNA Isolation

Total RNA extraction from tumor cell lines and patient samples is performed as described in Example 5.

### 2. Real-Time PCR

Real-time PCR and analysis of results are performed as shown in Example 2. ROC curves were prepared as described in Example 4.

### 3. Results

Increased levels of RNA expression are identified in colon tumor samples as compared to expression in normal colon samples. Normal colon samples show less RNA expression of SLC7A5 than do colon tumor samples. Level of the SLC7A5 biomarker mRNA was evaluated in a group of male colorectal cancer patients with stages ranging from Ito III. SLC7A5 is up-regulated significantly in colorectal cancer patients compared to the normal samples (FIG. 12).

SLC7A5 shows an 11.8-fold increase in the level of up-regulation relative to normal colon samples (Table 1).

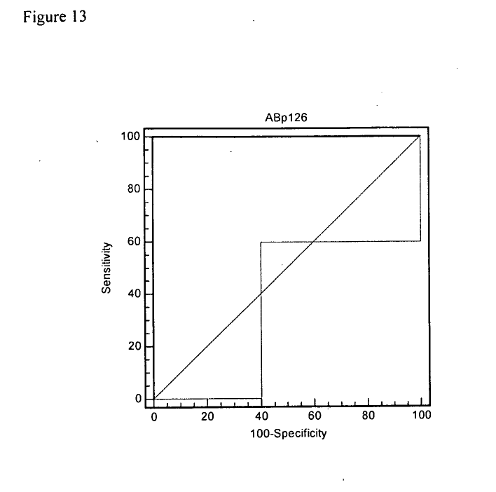

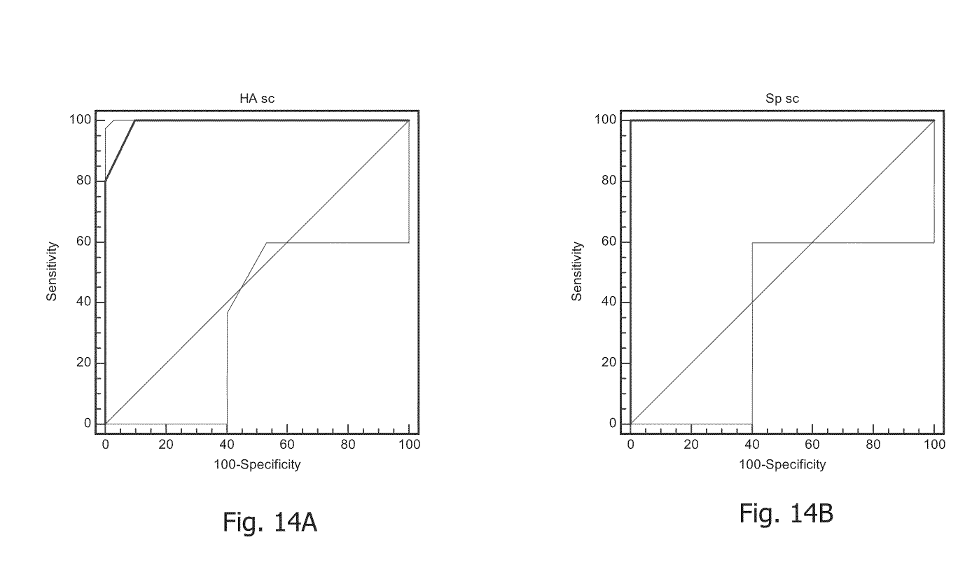

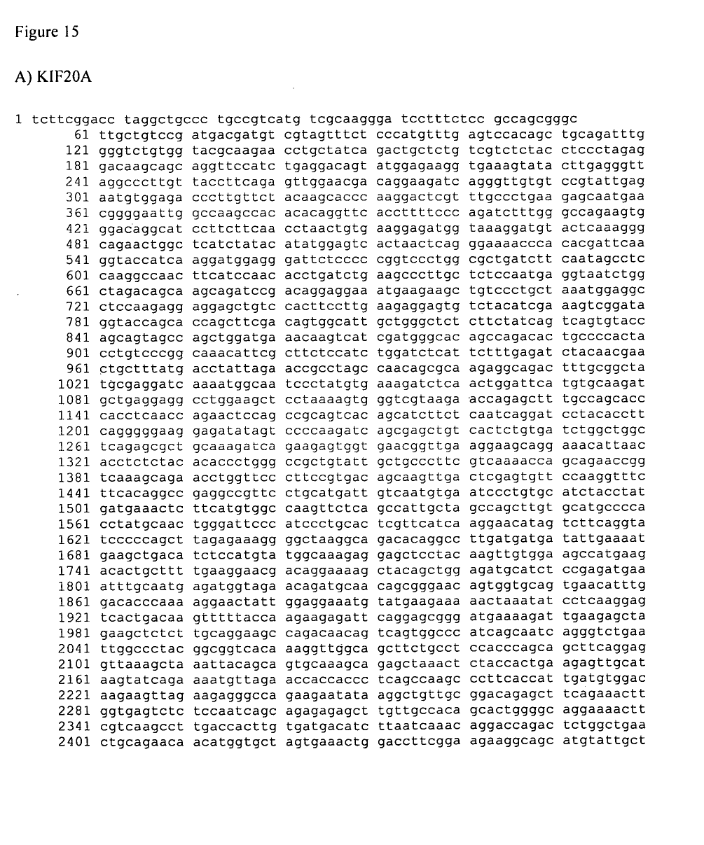

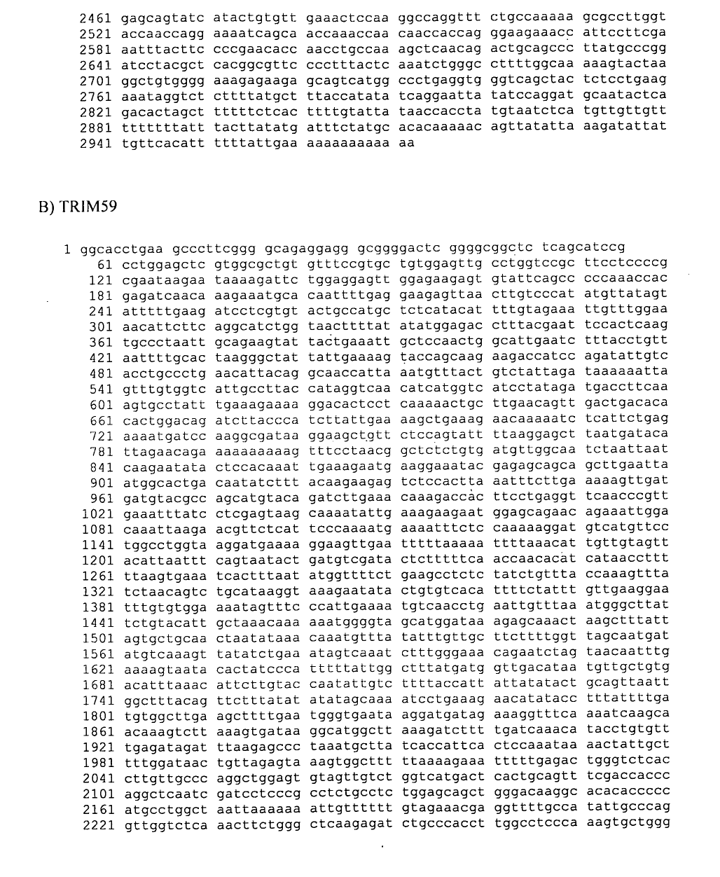

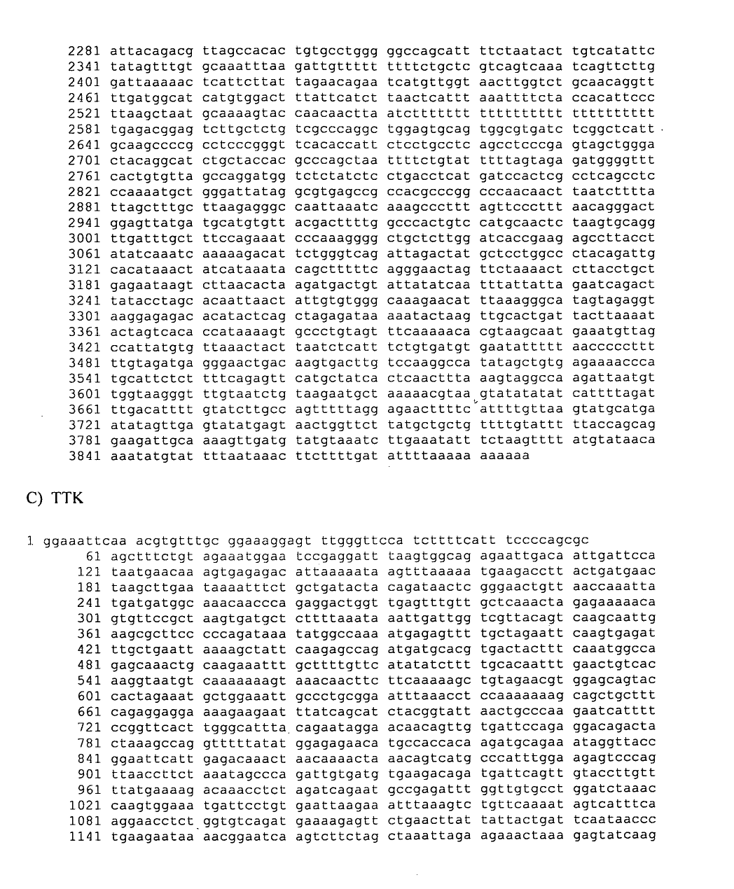

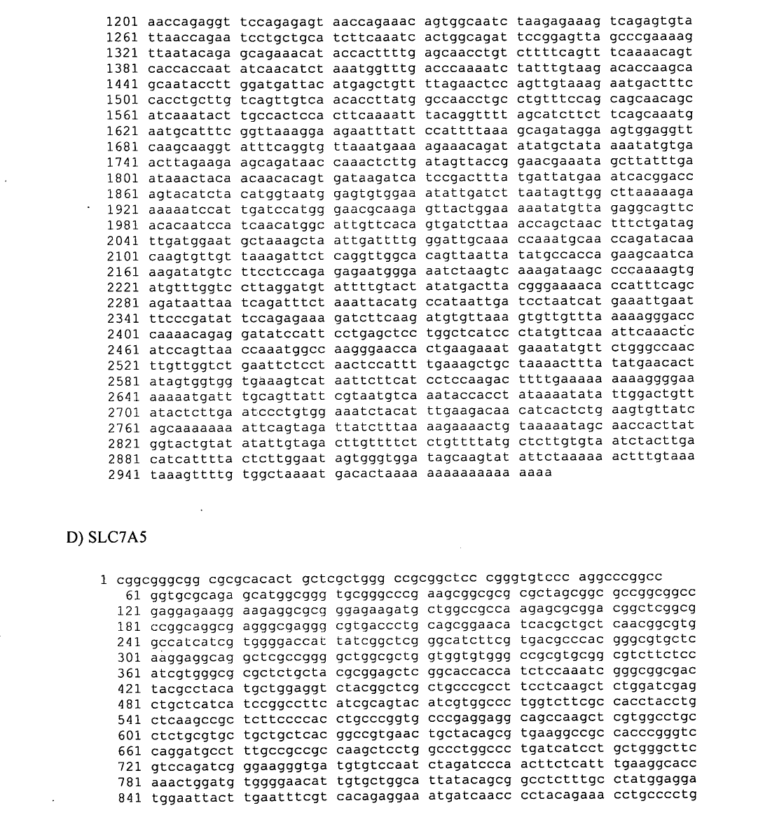

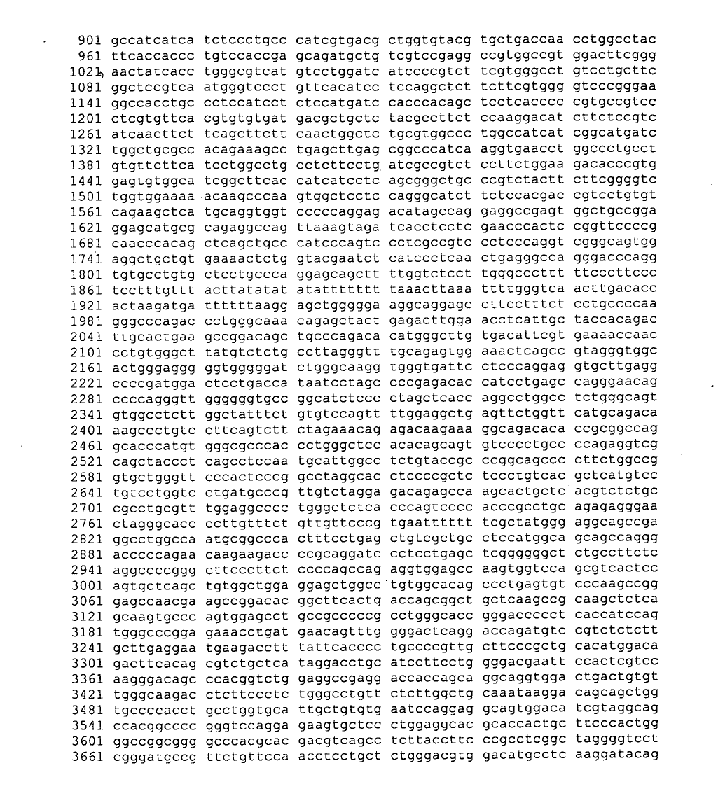

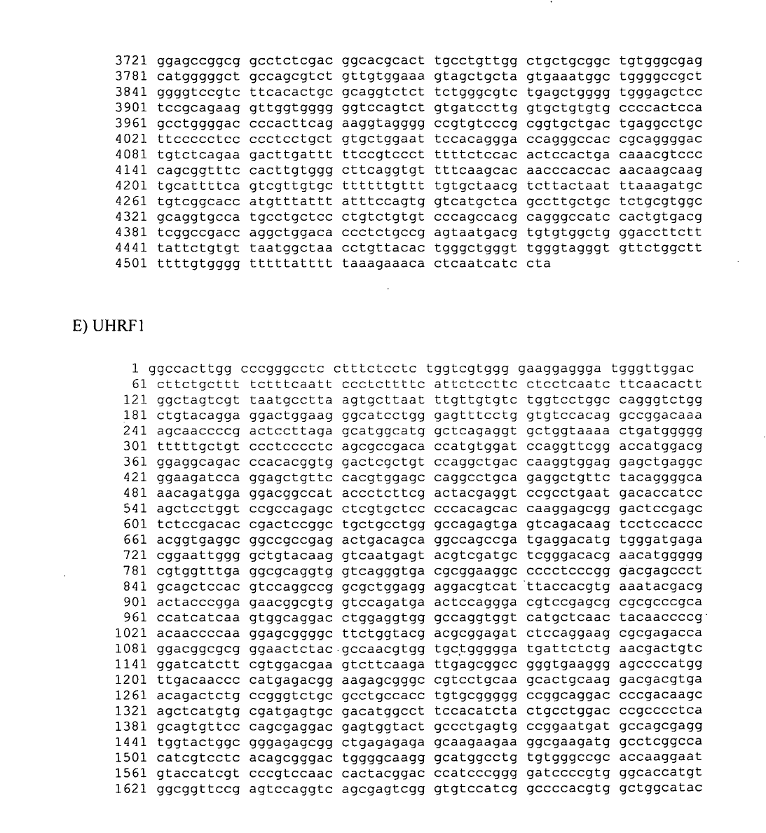

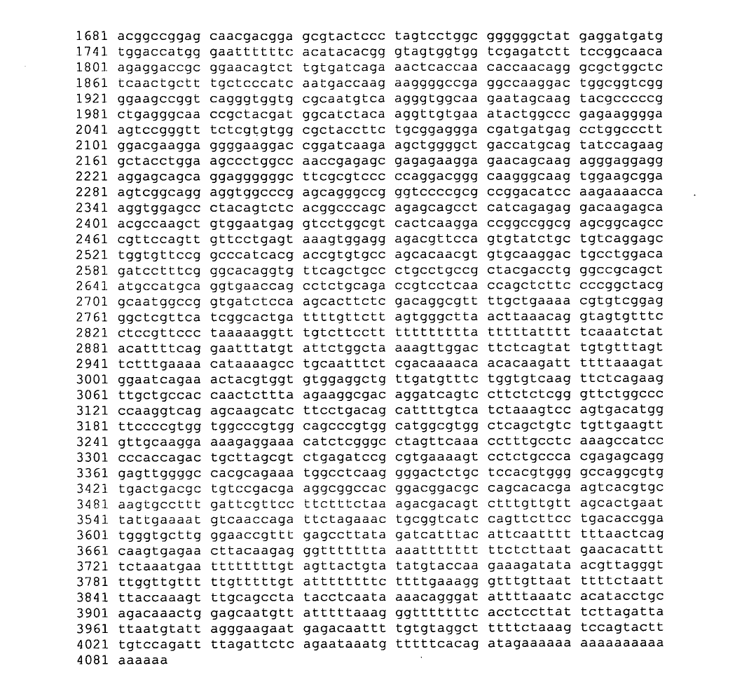

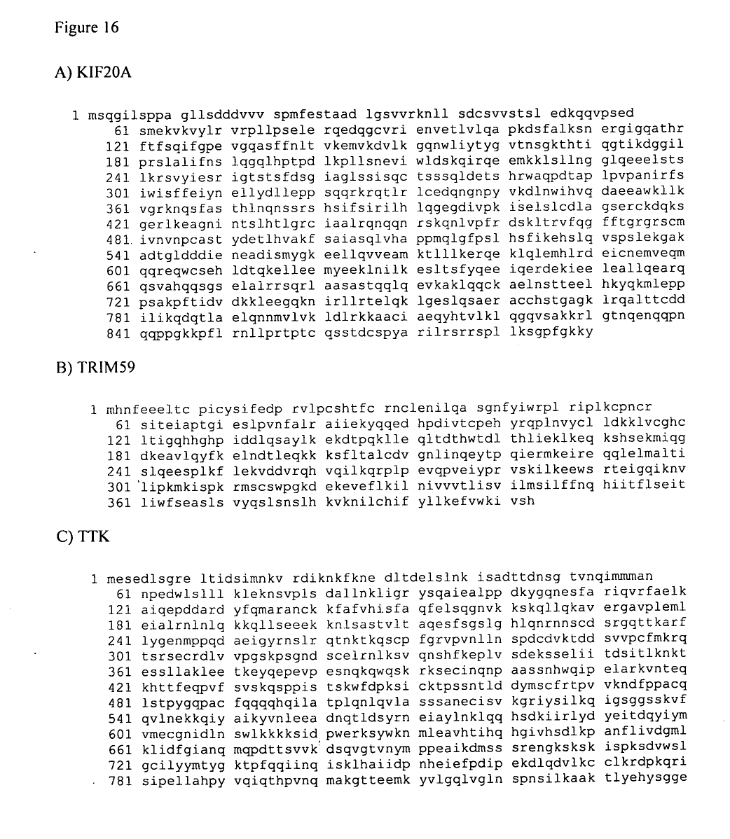

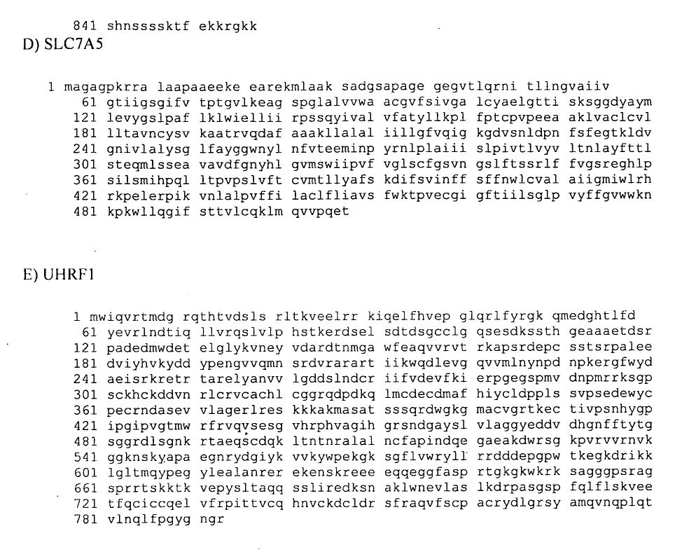

From ROC curves, it can be seen that the majority of the PAN biomarkers have good AUC separately (SLC7A5: FIG. 13) or in combination (Table 7).

TABLE 7

| | | High Accuracy | | | | 100% Specificity | | |
|---|---|---|---|---|---|---|---|---|
| Target | Auc | Sensitivity | Specificity | Cut-off | Auc | Sensitivity | Specificity | Cut-off |
| KIF20A | 0.94 | 88.2 | 90.9 | >0.21 | | 52.9 | 100 | >0..46 |
| SLC7A5 | 0.84 | 76.5 | 81.8 | >0..03 | | 29.4 | 100 | >0.13 |
| TRIM59 | 0.98 | 94.1 | 100.0 | >0.7 | | 94.1 | 100 | >0.7 |
| TTK | 0.995 | 94.1 | 100 | >0.1 | | 94.1 | 100 | >0.1 |
| UHRF1 | 0.85 | 100 | 72.7 | >0.009 | | 29.4 | 100 | >0.12 |
| KIF20A + SLC7A5 | 0.90 | 94.1 | 81.8 | >score 0 | 0.77 | 52.9 | 100 | >score 0 |
| KIF20A + TRIM59 | 0.97 | 88.2 | 100.0 | >score 1 | 0.97 | 94.1 | 100 | >score 0 |
| KIF20A + TTK | 0.97 | 88.2 | 100 | >score 1 | 0.97 | 94.1 | 100 | >score 0 |
| ABp125 + 129 | 0.84 | 88.2 | 82 | >score 0 | 0.79 | 58.8 | 100 | >score 0 |
| SLC7A5 + TRIM59 | 0.97 | 100.0 | 81.8 | >score 0 | 0.97 | 94.1 | 100 | >score 0 |
| SLC7A5 + TTK | 0.97 | 100 | 82 | >score 0 | 0.97 | 94 | 100 | >score 0 |
| SLC7A5 + UHRF1 | 0.79 | 88.2 | 72.7 | >score 0 | 0.91 | 81.8 | 100 | >score 0 |
| TRIM59 + TTK | 0.91 | 94.1 | 81.8 | >score 0 | 0.77 | 52.9 | 100 | >score 0 |
| TRIM59 + UHRF1 | 0.91 | 94 | 81.8 | >score 0 | 0.97 | 94.1 | 100 | >score 0 |
| TTK + UHRF1 | 0.91 | 94.1 | 82 | >score 0 | 0.97 | 94.1 | 100 | >score 0 |
| PAN (5) | 0.98 | 94 | 91 | >score 1 | 0.97 | 94.1 | 100 | >score 0 |
| Potentially Secreted (2) | 0.97 | 100 | 82 | >score 0 | 0.97 | 94.1 | 100 | >score 0 |

### Example 13

### Western Blot Analysis of Samples Isolated from Colon Cancer Patients and Normal Colon Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal colon and colon cancer samples, and are frozen into blocks of tissue. Protein cell extracts are then prepared from each block. Each patient included in the study is screened against the same normal total RNA group in order to compare them together. The tumor group is composed of at least 20 cases. The colon normal group is composed of at least 20 cases.

### 2. Western Blot Analysis of SLC7A5 in Colon Cancer and Colon Normal Samples

Colon cell samples are isolated and Western blot experiments are performed as described in Example 8.

### 3. Results

SLC7A5 expression is significantly increased in tumor samples obtained from colon tumor patients as compared to normal samples isolated from normal subjects. All normal subjects show nearly undetectable levels of SLC7A5 protein expression, while samples obtained from colon cancer patients show detectable levels of SLC7A5.

### Example 14

### ELISA Analysis of SLC7A5 in Colon Cancer and Colon Normal Tissues

### 1. Isolation and Preparation of Patient and Normal Tissues

Patient tissue samples are obtained and are prepared as described in Example 6.

### 2. ELISA Analysis

ELISA analysis is performed as described in Example 6.

### 3. Results

ELISA results show that samples from normal subjects expressed less SLC7A5 protein compared to colon cancer patient samples. These results confirm the results obtained in the Western blot analysis.

Combinations biomarkers did not significantly improve the predictive power of these biomarkers, except for TTK and SLC7A5 which had an AUC of 85% and 50% sensitivity. However, when all biomarkers are combined together, the AUC is 86%; with a min. requirement of three biomarkers with normalized qPCR ratios higher than their cut-off values (Table 7). The secreted biomarkers have an AUC of 78% but a specificity of only 40% (Table 7).

### Example 15

### Western Blot Analysis of a Samples Isolated From Leukemia Patients and Normal Subjects

### 1. Patient Samples and Normal Samples

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient sample included in the study is screened against the same normal total RNA group in order to compare them together.

### 2. Western Blot Analysis of SLC7A5 in Leukemia and Normal Samples

Blood samples are prepared by isolating blood from leukemia patients. The blood samples are fractioned initially to isolate remove red-blood cells. The samples containing all white blood cell are further fractionated by FACS sorting based on size defractions and/or using surface specific monoclonal antibodies. Purified cells are then lysed in lysis buffer as described in the above examples. Quantified cell lysates from leukemia samples and normal blood cells are then resolved on SDS-PAGE and prepared for Western blotting to probe for SLC7A5 and other biomarkers.

### 3. Results

SLC7A5 expression is significantly increased in cell and fluid samples obtained from leukemia patients as compared to expression in cell and fluid samples isolated from normal subjects. All normal subjects show nearly undetectable or nearly undetectable levels of SLC7A5 protein expression, while samples obtained from leukemia patients show detectable levels of SLC7A5.

### Example 16

### Preparation and Use of Focused Microarray to Detect SLC7A5 in Samples Obtained From Normal Subjects and Leukemia Patients

1. Total RNA Isolation and cDNA Labeling

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

Blood samples are prepared as described in Example 16. For leukemia tissue samples, human marrow tissues are homogenized and prepared for analysis following procedures described in Example 7.

First strand cDNA labeling, cDNA digestion, capture probe preparation and focused microarray preparation are accomplished using procedures described in Example 1. In addition, quality control and focused microarray hybridization are performed according to procedures described in Example 1. The QuantArray® data results are analyzed according to the procedures described above in Example 1.

### 2. Results

SLC7A5 mRNA expression correlates with SLC7A5 protein expression. Increased levels of SLC7A5 mRNA are detected in cell and fluid samples obtained patients suffering from leukemia compared to expression in samples from normal subjects. Cell and fluid samples from patients suffering from leukemia have higher levels of SLC7A5 mRNA expression than do samples from normal subjects.

### Example 17

### Western Blot Analysis of Samples Isolated From Sarcoma Patients and Normal Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal sarcoma and sarcoma cancer samples, and are frozen into blocks of tissue. Protein cell extracts are then prepared from each block. Each patient included in the study is screened against the same normal total RNA group in order to compare them together. The tumor group is composed of at least 20 cases. The normal prostate group is composed of at least 20 cases.

### 2. Western Blot Analysis of SLC7A5 in Sarcoma Cancer and Normal Samples

Sample preparation and Western blot analysis are performed as described in Example 8.

### 3. Results

SLC7A5 expression is increased in tumor samples obtained from sarcoma tumor patients compared to expression in control samples isolated from normal subjects. All normal subjects show nearly undetectable or undetectable levels of SLC7A5 protein expression, while samples obtained from sarcoma cancer patients show detectable levels of SLC7A5.

### Example 18

### ELISA Analysis of SLC7A5 in Sarcoma Cancer and Normal Tissues

### 1. Isolation and Preparation of Patient and Normal Tissues

Patient tissue samples are obtained and are prepared as described in Example 6.

### 2. ELISA Analysis

ELISA analysis is performed as described in Example 6.

### 3. Results

ELISA results show that samples from normal subjects expressed less SLC7A5 protein compared to samples from sarcoma cancer patients. These results confirm the results obtained by the Western blot analysis.

### Example 19

### Preparation and Use the Focused Microarray to Detect SLC7A5 in Samples Obtained From Normal Sarcoma Subjects and Sarcoma Cancer Patients

1. Total RNA Isolation and cDNA Labeling

Patient Sarcoma tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Capture Probe and Focused Microarray Preparation

Capture probe preparation and printing of capture probes are performed according to the procedure provided in Example 12. The preparation of the microarray, quality control, hybridization, and analysis of the results are performed as described in Example 11.

### 3. Results

SLC7A5 mRNA expression correlates with SLC7A5 protein expression. Increased levels of SLC7A5 mRNA are detected in cell sample obtained patients suffering from sarcoma cancer compared to expression in samples from normal subjects. Cell samples from patients suffering from sarcoma cancer have higher levels of SLC7A5 mRNA expression than do normal subjects.

### Example 20

### Real-Time PCR Analysis of Samples Isolated From Sarcoma Cancer Patients and Normal Sarcoma Subjects

### 1. Patient Samples and RNA Isolation

Total RNA extraction from tumor cell lines and patient samples is performed as described in Example 5.

### 2. Real-Time PCR

Real-time PCR and analysis of results are performed as shown in Example 3.

### 3. Results

Increased levels of RNA expression are identified in colon tumor samples compared to normal colon samples. Normal sarcoma samples show less RNA expression of SLC7A5 than do sarcoma tumor samples. These results confirm the results obtained from the microarray experiments described in Example 23.

### Example 21

### Western Blot Analysis of Samples Isolated From Melanoma Patients and Normal Subjects

### 1. Patient Samples and Normal Samples

Patient tissues and fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Western Blot Analysis of SLC7A5 in Melanoma and Normal Samples

Sample preparation and Western blot analysis are performed as described in Example 9.

### 3. Results

SLC7A5 expression is increased in samples obtained from melanoma tumor patients compared to samples isolated from normal subjects. All normal subjects show undetectable or nearly undetectable levels of SLC7A5 protein expression, while samples obtained from melanoma cancer patients show detectable levels of SLC7A5.

### Example 22

### ELISA Analysis of SLC7A5 in Melanoma Cancer and Melanoma Normal Tissues

### 1. Isolation and Preparation of Patient and Normal Tissues

Patient tissue samples are obtained and are prepared as described in Example 6.

### 2. ELISA Analysis

ELISA analysis is performed as described in Example 6.

### 3. Results

ELISA results show that normal subjects expressed less SLC7A5 protein compared to melanoma cancer patient samples. These results confirm the results obtained in the Western blot analysis.

### Example 23

### Preparation and Use of Focused Microarray to Detect SLC7A5 in Samples Obtained From Normal Melanoma Subjects and Melanoma Cancer Patients

1. Total RNA Isolation and cDNA Labeling

Patient Melanoma tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA group in order to compare them together.

### 2. Capture Probe Preparation and Focused Microarray Preparation

Capture probe preparation and printing of capture probes are performed according to the procedure provided in Example 12. The preparation of the microarray, quality control, hybridization, and analysis of the results is performed as detailed in Example 11.

### 3. Results

SLC7A5 mRNA expression correlates with SLC7A5 protein expression. Increased levels of SLC7A5 mRNA are detected in cell obtained patients suffering from melanoma cancer compared to normal subjects. Cell samples from patients suffering from melanoma cancer have higher levels of SLC7A5 mRNA expression than are found in samples from normal subjects.

### Example 24

### Real-Time PCR Analysis of Samples Isolated From Melanoma Cancer Patients and Normal Melanoma Subjects

### 1. Patient Samples and RNA Isolation

Total RNA extraction from tumor cell lines and patient samples is performed as described in Example 5.

### 2. Real-Time PCR

Real-time PCR and analysis of results is performed as described in Example 3.

### 3. Results

Increased levels of RNA expression are identified in colon tumor samples compared to expression in normal colon samples. Normal melanoma samples show less SLC7A5 RNA expression than do melanoma tumor samples. These results confirm the results obtained from the microarray experiments described in Example 24.

### EQUIVALENTS

Those skilled in the art will recognize, or be able to ascertain, using no more than routine experimentation, numerous equivalents to the specific compositions and procedures described herein. Such equivalents are considered to be within the scope of this disclosure, and are covered by the following claims.

1. A method for detecting a neoplasm comprising:
a) obtaining a potentially neoplastic test sample and a corresponding non-neoplastic control sample;
b) b) detecting a level of SLC7A5 expression in the test sample and in the control sample; and
comparing the level of SLC7A5 expression in the test sample to the level of SLC7A5 expression in the control sample,
the test sample being neoplastic if the level of SLC7A5 expression in the test sample is detectably greater than the level of SLC7A5 expression in the control sample.

2. The method of claim 1, wherein the neoplastic test sample and the control samples are cell samples of the same lineage.

3. The method of claim 2, wherein detecting the level of expression of SLC7A5 comprises isolating a cytoplasmic fraction from the test cell sample and from the control cell sample, and then separately detecting the level of expression of SLC7A5 in these cytoplasmic fractions.

4. The method of claim 1, wherein the level of expression of SLC7A5 protein is detected by contacting the test sample and the control sample with a SLC7A5-specific protein binding agent selected from the group consisting of an anti-SLC7A5 antibody, SLC7A5-binding portions of an antibody, SLC7A5-specific ligands, SLC7A5-specific aptamers, and SLC7A5 inhibitors.

5. The method of claim 4, wherein the SLC7A5-specific protein binding agent is immobilized on a solid support.

6. The method of claim 1, wherein SLC7A5 expression is detected by detecting the level of expression of SLC7A5 RNA by contacting the test sample and the control sample with a SLC7A5 RNA-specific nucleic acid binding agent and determining how much of the nucleic acid binding agent is hybridized to SLC7A5 RNA in the test sample and in the control sample.

7. The method of claim 6, wherein the nucleic acid binding agent is immobilized on a solid support.

8. The method of claim 1, wherein the level of expression of SLC7A5 in the test sample is at least 1.5, at least 2, at least 4, at least 6, at least 8, at least 10, or at least 20 times greater than the level of expression of SLC7A5 in the control sample.

9. The method of claim 1, wherein the test sample is isolated from a patient suffering from ovarian cancer.

10. The method of claim 1, wherein the test sample is isolated from a patient suffering from breast cancer.

11. The method of claim 1, wherein the test sample is isolated from a patient suffering from colon cancer.

12. The method of claim 1, wherein the test sample is isolated from a patient suffering from lung cancer.

13. The method of claim 1, wherein the test sample is isolated from a patient suffering from melanoma.

14. The method of claim 1, wherein the test sample is isolated from a patient suffering from sarcoma.

15. The method of claim 1, wherein the test sample is isolated from a patient suffering from leukemia.

16. The method of claim 1, wherein the test sample and the control samples are fluid samples.

17. The method of claim 16, wherein the level of SLC7A5 protein expression is determined by measuring the level of anti-SLC7A5 antibody in the test fluid sample and in the control fluid sample.

18. The method of claim 16, wherein the test and control fluid samples are serum samples.

19. The method of claim 16, wherein the level of expression of anti-SLC7A5 antibody is detected with an anti-SLC7A5 antibody-specific antibody, or anti-SLC7A5 antibody-specific antibody fragment thereof.

20. A method for detecting a neoplasm comprising:
a) obtaining a potentially neoplastic test sample and a non-neoplastic control sample;
b) detecting a level of SLC7A5 expression in the test sample and in the control sample;
c) detecting a level of expression of at least one of TRIM59, TTK, UHRF1, and/or KIF20A; and
d) comparing the level of SLC7A5 expression and the level of expression of at least one of TTK, UHRF1, TRIM59 and/or KIF20A in the test sample to the level of SLC7A5 expression and the level of expression of the at least one of TTK, UHRF1, TRIM59 and/or KIF20A in the control sample,
the test sample being neoplastic if the levels of expression of SLC7A5 and the at least one of TTK, UHRF1, TRIM59 and/or KIF20 in the test sample are detectably greater than the levels of expression of SLC7A5 and the at least one of TTK, UHRF1, TRIM59 and/or KIF20A in the control sample.

21. The method of claim 20, wherein detecting step (c) comprises detecting the level of at least TRIM59 expression, and step (d) comprises comparing the level of SLC7A5 expression and at least TRIM59 expression in the test and control samples.

22. The method of claim 20, wherein the level of SLC7A5 expression is detected by contacting the test sample and the control sample with a SLC7A5-specific protein binding agent selected from the group consisting of an SLC7A5-specific antibody, SLC7A5-specific binding portions of an antibody, a SLC7A5-specific ligand, a SLC7A5-specific aptamer, and an SLC7A5 inhibitor.

23. The method of claim 22, wherein the SLC7A5-specific protein binding agent is immobilized on a solid support.

24. The method of claim 20, wherein the level of expression of SLC7A5 in the test and control samples is measured by measuring the level of SLC7A5 RNA and the level of at least one of TTK RNA, UHRF1 RNA, TRIM59 RNA, and/or KIF20A RNA in the test and control samples.

25. The method of claim 24, wherein the level of expression of SLC7A5 RNA and the level of expression of at least one of TTK RNA, UHRF1 RNA, TRIM59 RNA, and/or KIF20A RNA are detected by contacting the test sample and the control sample with an TTK-specific nucleic acid binding agent and with at least one of a TTK-specific nucleic acid binding agent, a UHRF1-specific nucleic binding agent, a TTK-specific nucleic acid binding agent, and a KIF20A-specific nucleic acid binding agent.

26. The method of claim 20, wherein the levels of expression of SLC7A5, and the levels of expression of TTK, UHRF1, TRIM59 and/or KIF20 in the test sample are at least 1.5 times greater than the level of expression of SLC7A5, TTK, UHRF1, TRIM59, and/or KIF20 in the control sample.

27. The method of claim 20, wherein the test and control samples are cell samples.

28. The method of claim 27, wherein detecting the level of expression of SLC7A5 and the level of expression of at least one of TTK, UHRF1, TRIM59 and/or KIF20A comprises isolating a cytoplasmic fraction from the test cell sample and from the control cell sample, and then detecting the levels of expression of SLC7A5 and at least one of TTK, UHRF1, TR1M59 and/or KIF20A in each of these cytoplasmic fractions.

29. The method of claim 20, wherein the test and control samples are fluid samples.

30. The method of claim 20, wherein the level of expression of SLC7A5 is measured by detecting a level of anti-SLC7A5 antibody in a test fluid sample and in a control fluid sample.

31. The method of claim 20, wherein the test sample is isolated from a tissue of a patient suffering from ovarian cancer, breast cancer, lung cancer, sarcoma, melanoma, or leukemia.

32. The method of claim 20, wherein detecting step (c) comprises detecting the expression of at least TRIM 59.

33. A kit for diagnosing or detecting neoplasia, comprising:
a) a first probe specific for the detection of SLC7A5; and
b) a second probe specific for the detection of a neoplasia marker selected from the group consisting of TTK, UHRF1, TRIM59, KIF20A, and combinations thereof,

34. A method of treating a neoplasm in a patient, comprising:
(a) administering a therapeutically effective amount of an SLC7A5-specific antibody, or SLC7A5-specific binding fragment thereof, to a patient in need thereof; and
(b) detecting a decrease in the presence of the neoplasm, or symptoms resulting from the neoplasm.

35. A cell surface SLC7A5-targeted agent for treating a neoplastic cell growth, comprising:
(a) a SLC7A5 binding component; and
(b) a therapeutic component,
the SLC7A5 binding component targeting the therapeutic component to the neoplastic cell growth, thereby treating the cancer.

36. A method of treating a neoplasm in a subject, comprising administering the cell surface SLC7A5-targeted agent of claim 35.

37. A vaccine for treating neoplasm, comprising:
(a) a SLC7A5 polypeptide, or SLC7A5 polypeptide subsequence thereof; and
(b) at least one pharmaceutically acceptable vaccine component.

38. The vaccine of claim 37, wherein the SLC7A5 polypeptide or polypeptide subsequence is a human SLC7A5 polypeptide sequence having an amino acid sequence of SEQ ID NO:4


SEQUENCE LISTING (13 sequences)

SEQ ID NO: 1 - protein, 890 aa, Homo sapiens
MSQGILSPPAGLLSDDDVVVSPMFESTAADLGSVVRKNLLSDCSVVSTSLEDKQQVPSEDSMEKVKVYLRVRPLLPSELERQEDQGCVRIENVETLVLQAPKDSFALKSNERGIGQATHRFTFSQIFGPEVGQASFFNLTVKEMVKDVLKGQNWLIYTYGVTNSGKTHTIQGTIKDGGILPRSLALIFNSLQGQLHPTPDLKPLLSNEVIWLDSKQIRQEEMKKLSLLNGGLQEEELSTSLKRSVYIESRIGTSTSFDSGIAGLSSISQCTSSSQLDETSHRWAQPDTAPLPVPANIRFSIWISFFEIYNELLYDLLEPPSQQRKRQTLRLCEDQNGNPYVKDLNWIHVQDAEEAWKLLKVGRKNQSFASTHLNQNSSRSHSIFSIRILHLQGEGDIVPKISELSLCDLAGSERCKDQKSGERLKEAGNINTSLHTLGRCIAALRQNQQNRSKQNLVPFRDSKLTRVFQGFFTGRGRSCMIVNVNPCASTYDETLHVAKFSAIASQLVHAPPMQLGFPSLHSFIKEHSLQVSPSLEKGAKADTGLDDDIENEADISMYGKEELLQVVEAMKTLLLKERQEKLQLEMHLRDEICNEMVEQMQQREQWCSEHLDTQKELLEEMYEEKLNILKESLTSFYQEEIQERDEKIEELEALLQEARQQSVAHQQSGSELALRRSQRLAASASTQQLQEVKAKLQQCKAELNSTTEELHKYQKMLEPPPSAKPFTIDVDKKLEEGQKNIRLLRTELQKLGESLQSAERACCHSTGAGKLRQALTTCDDILIKQDQTLAELQNNMVLVKLDLRKKAACIAEQYHTVLKLQGQVSAKKRLGTNQENQQPNQQPPGKKPFLRNLLPRTPTCQSSTDCSPYARILRSRRSPLLKSGPFGKKY

SEQ ID NO: 2 - protein, 403 aa, Homo sapiens
MHNFEEELTCPICYSIFEDPRVLPCSHTFCRNCLENILQASGNFYIWRPLRIPLKCPNCRSITEIAPTGIESLPVNFALRAIIEKYQQEDHPDIVTCPEHYRQPLNVYCLLDKKLVCGHCLTIGQHHGHPIDDLQSAYLKEKDTPQKLLEQLTDTHWTDLTHLIEKLKEQKSHSEKMIQGDKEAVLQYFKELNDTLEQKKKSFLTALCDVGNLINQEYTPQIERMKEIREQQLELMALTISLQEESPLKFLEKVDDVRQHVQILKQRPLPEVQPVEIYPRVSKILKEEWSRTEIGQIKNVLIPKMKISPKRMSCSWPGKDEKEVEFLKILNIVVVTLISVILMSILFFNQHIITFLSEITLIWFSEASLSVYQSLSNSLHKVKNILCHIFYLLKEFVWKIVSH

SEQ ID NO: 3 - protein, 857 aa, Homo sapiens
MESEDLSGRELTIDSIMNKVRDIKNKFKNEDLTDELSLNKISADTTDNSGTVNQIMMMANNPEDWLSLLLKLEKNSVPLSDALLNKLIGRYSQAIEALPPDKYGQNESFARIQVRFAELKAIQEPDDARDYFQMARANCKKFAFVHISFAQFELSQGNVKKSKQLLQKAVERGAVPLEMLEIALRNLNLQKKQLLSEEEKKNLSASTVLTAQESFSGSLGHLQNRNNSCDSRGQTTKARFLYGENMPPQDAEIGYRNSLRQTNKTKQSCPFGRVPVNLLNSPDCDVKTDDSVVPCFMKRQTSRSECRDLVVPGSKPSGNDSCELRNLKSVQNSHFKEPLVSDEKSSELIITDSITLKNKTESSLLAKLEETKEYQEPEVPESNQKQWQSKRKSECINQNPAASSNHWQIPELARKVNTEQKHTTFEQPVFSVSKQSPPISTSKWFDPKSICKTPSSNTLDDYMSCFRTPVVKNDFPPACQLSTPYGQPACFQQQQHQILATPLQNLQVLASSSANECISVKGRIYSILKQIGSGGSSKVFQVLNEKKQIYAIKYVNLEEADNQTLDSYRNEIAYLNKLQQHSDKIIRLYDYEITDQYIYMVMECGNIDLNSWLKKKKSIDPWERKSYWKNMLEAVHTIHQHGIVHSDLKPANFLIVDGMLKLIDFGIANQMQPDTTSVVKDSQVGTVNYMPPEAIKDMSSSRENGKSKSKISPKSDVWSLGCILYYMTYGKTPFQQIINQISKLHAIIDPNHEIEFPDIPEKDLQDVLKCCLKRDPKQRISIPELLAHPYVQIQTHPVNQMAKGTTEEMKYVLGQLVGLNSPNSILKAAKTLYEHYSGGESHNSSSSKTFEKKRGKK

SEQ ID NO: 4 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 5 - protein, 793 aa, Homo sapiens
MWIQVRTMDGRQTHTVDSLSRLTKVEELRRKIQELFHVEPGLQRLFYRGKQMEDGHTLFDYEVRLNDTIQLLVRQSLVLPHSTKERDSELSDTDSGCCLGQSESDKSSTHGEAAAETDSRPADEDMWDETELGLYKVNEYVDARDTNMGAWFEAQVVRVTRKAPSRDEPCSSTSRPALEEDVIYHVKYDDYPENGVVQMNSRDVRARARTIIKWQDLEVGQVVMLNYNPDNPKERGFWYDAEISRKRETRTARELYANVVLGDDSLNDCRIIFVDEVFKIERPGEGSPMVDNPMRRKSGPSCKHCKDDVNRLCRVCACHLCGGRQDPDKQLMCDECDMAFHIYCLDPPLSSVPSEDEWYCPECRNDASEVVLAGERLRESKKKAKMASATSSSQRDWGKGMACVGRTKECTIVPSNHYGPIPGIPVGTMWRFRVQVSESGVHRPHVAGIHGRSNDGAYSLVLAGGYEDDVDHGNFFTYTGSGGRDLSGNKRTAEQSCDQKLTNTNRALALNCFAPINDQEGAEAKDWRSGKPVRVVRNVKGGKNSKYAPAEGNRYDGIYKVVKYWPEKGKSGFLVWRYLLRRDDDEPGPWTKEGKDRIKKLGLTMQYPEGYLEALANREREKENSKREEEEQQEGGFASPRTGKGKWKRKSAGGGPSRAGSPRRTSKKTKVEPYSLTAQQSSLIREDKSNAKLWNEVLASLKDRPASGSPFQLFLSKVEETFQCICCQELVFRPITTVCQHNVCKDCLDRSFRAQVFSCPACRYDLGRSYAMQVNQPLQTVLNQLFPGYGNGR

SEQ ID NO: 6 - DNA, 2972 bp, Homo sapiens
tcttcggacctaggctgccctgccgtcatgtcgcaagggatcctttctccgccagcgggcttgctgtccgatgacgatgtcgtagtttctcccatgtttgagtccacagctgcagatttggggtctgtggtacgcaagaacctgctatcagactgctctgtcgtctctacctccctagaggacaagcagcaggttccatctgaggacagtatggagaaggtgaaagtatacttgagggttaggcccttgttaccttcagagttggaacgacaggaagatcagggttgtgtccgtattgagaatgtggagacccttgttctacaagcacccaaggactcgtttgccctgaagagcaatgaacggggaattggccaagccacacacaggttcaccttttcccagatctttgggccagaagtgggacaggcatccttcttcaacctaactgtgaaggagatggtaaaggatgtactcaaagggcagaactggctcatctatacatatggagtcactaactcagggaaaacccacacgattcaaggtaccatcaaggatggagggattctcccccggtccctggcgctgatcttcaatagcctccaaggccaacttcatccaacacctgatctgaagcccttgctctccaatgaggtaatctggctagacagcaagcagatccgacaggaggaaatgaagaagctgtccctgctaaatggaggcctccaagaggaggagctgtccacttccttgaagaggagtgtctacatcgaaagtcggataggtaccagcaccagcttcgacagtggcattgctgggctctcttctatcagtcagtgtaccagcagtagccagctggatgaaacaagtcatcgatgggcacagccagacactgccccactacctgtcccggcaaacattcgcttctccatctggatctcattctttgagatctacaacgaactgctttatgacctattagaaccgcctagccaacagcgcaagaggcagactttgcggctatgcgaggatcaaaatggcaatccctatgtgaaagatctcaactggattcatgtgcaagatgctgaggaggcctggaagctcctaaaagtgggtcgtaagaaccagagctttgccagcacccacctcaaccagaactccagccgcagtcacagcatcttctcaatcaggatcctacaccttcagggggaaggagatatagtccccaagatcagcgagctgtcactctgtgatctggctggctcagagcgctgcaaagatcagaagagtggtgaacggttgaaggaagcaggaaacattaacacctctctacacaccctgggccgctgtattgctgcccttcgtcaaaaccagcagaaccggtcaaagcagaacctggttcccttccgtgacagcaagttgactcgagtgttccaaggtttcttcacaggccgaggccgttcctgcatgattgtcaatgtgaatccctgtgcatctacctatgatgaaactcttcatgtggccaagttctcagccattgctagccagcttgtgcatgccccacctatgcaactgggattcccatccctgcactcgttcatcaaggaacatagtcttcaggtatcccccagcttagagaaaggggctaaggcagacacaggccttgatgatgatattgaaaatgaagctgacatctccatgtatggcaaagaggagctcctacaagttgtggaagccatgaagacactgcttttgaaggaacgacaggaaaagctacagctggagatgcatctccgagatgaaatttgcaatgagatggtagaacagatgcaacagcgggaacagtggtgcagtgaacatttggacacccaaaaggaactattggaggaaatgtatgaagaaaaactaaatatcctcaaggagtcactgacaagtttttaccaagaagagattcaggagcgggatgaaaagattgaagagctagaagctctcttgcaggaagccagacaacagtcagtggcccatcagcaatcagggtctgaattggccctacggcggtcacaaaggttggcagcttctgcctccacccagcagcttcaggaggttaaagctaaattacagcagtgcaaagcagagctaaactctaccactgaagagttgcataagtatcagaaaatgttagaaccaccaccctcagccaagcccttcaccattgatgtggacaagaagttagaagagggccagaagaatataaggctgttgcggacagagcttcagaaacttggtgagtctctccaatcagcagagagagcttgttgccacagcactggggcaggaaaacttcgtcaagccttgaccacttgtgatgacatcttaatcaaacaggaccagactctggctgaactgcagaacaacatggtgctagtgaaactggaccttcggaagaaggcagcatgtattgctgagcagtatcatactgtgttgaaactccaaggccaggtttctgccaaaaagcgccttggtaccaaccaggaaaatcagcaaccaaaccaacaaccaccagggaagaaaccattccttcgaaatttacttccccgaacaccaacctgccaaagctcaacagactgcagcccttatgcccggatcctacgctcacggcgttcccctttactcaaatctgggccttttggcaaaaagtactaaggctgtggggaaagagaagagcagtcatggccctgaggtgggtcagctactctcctgaagaaataggtctcttttatgctttaccatatatcaggaattatatccaggatgcaatactcagacactagcttttttctcacttttgtattataaccacctatgtaatctcatgttgttgtttttttttatttacttatatgatttctatgcacacaaaaacagttatattaaagatattattgttcacattttttattgaaaaaaaaaaaaaa

SEQ ID NO: 7 - DNA, 3886 bp, Homo sapiens
ggcacctgaagcccttcggggcagaggagggcggggactcggggcggctctcagcatccgcctggagctcgtggcgctgtgtttccgtgctgtggagttgcctggtccgcttcctccccgcgaataagaataaaagattctggaggagttggagaagagtgtattcagcccccaaaccacgagatcaacaaagaaatgcacaattttgaggaagagttaacttgtcccatatgttatagtatttttgaagatcctcgtgtactgccatgctctcatacattttgtagaaattgtttggaaaacattcttcaggcatctggtaacttttatatatggagacctttacgaattccactcaagtgccctaattgcagaagtattactgaaattgctccaactggcattgaatctttacctgttaattttgcactaagggctattattgaaaagtaccagcaagaagaccatccagatattgtcacctgccctgaacattacaggcaaccattaaatgtttactgtctattagataaaaaattagtttgtggtcattgccttaccataggtcaacatcatggtcatcctatagatgaccttcaaagtgcctatttgaaagaaaaggacactcctcaaaaactgcttgaacagttgactgacacacactggacagatcttacccatcttattgaaaagctgaaagaacaaaaatctcattctgagaaaatgatccaaggcgataaggaagctgttctccagtattttaaggagcttaatgatacattagaacagaaaaaaaaaagtttcctaacggctctctgtgatgttggcaatctaattaatcaagaatatactccacaaattgaaagaatgaaggaaatacgagagcagcagcttgaattaatggcactgacaatatctttacaagaagagtctccacttaaatttcttgaaaaagttgatgatgtacgccagcatgtacagatcttgaaacaaagaccacttcctgaggttcaacccgttgaaatttatcctcgagtaagcaaaatattgaaagaagaatggagcagaacagaaattggacaaattaagaacgttctcattcccaaaatgaaaatttctccaaaaaggatgtcatgttcctggcctggtaaggatgaaaaggaagttgaatttttaaaaattttaaacattgttgtagttacattaatttcagtaatactgatgtcgatactctttttcaaccaacacatcataacctttttaagtgaaatcactttaatatggttttctgaagcctctctatctgtttaccaaagtttatctaacagtctgcataaggtaaagaatatactgtgtcacattttctatttgttgaaggaatttgtgtggaaaatagtttcccattgaaaatgtcaacctgaattgtttaaatgggcttattctgtacattgctaaacaaaaaatggggtagcatggataaagagcaaactaagctttattagtgctgcaactaatataaacaaatgtttatatttgttgcttcttttggttagcaatgatatgtcaaagttatatctgaaatagtcaaatctttgggaaacagaatctagtaacaatttgaaaagtaatacactatcccatttttattggctttatgatggttgacataatgttgctgtgacatttaaacattcttgtaccaatattgtcttttaccattattatatactgcagttaattggctttacagttctttatatatatagcaaaatcctgaaagaacatatacctttattttgatgtggcttgaagcttttgaatgggtgaataaggatgatagaaaggtttcaaaatcaagcaacaaagtcttaaagtgataaggcatggcttaaagatcttttgatcaaacatacctgtgtttgagatagatttaagagccctaaatgcttatcaccattcactccaaataaaactattgcttttggataactgttagagtaaagtggctttttaaaagaaatttttgagactgggtctcaccttgttgcccaggctggagtgtagttgtctggtcatgactcactgcagtttcgaccacccaggctcaatcgatcctcccgcctctgcctctggagcagctgggacaaggcacacacccccatgcctggctaattaaaaaaattgttttttgtagaaacgaggttttgccatattgcccaggttggtctcaaacttctgggctcaagagatctgcccaccttggcctcccaaagtgctgggattacagacgttagccacactgtgcctgggggccagcattttctaatacttgtcatattctatagtttgtgcaaatttaagattgtttttttttctgctcgtcagtcaaatcagttcttggattaaaaactcattcttattagaacagaatcatgttggtaacttggtctgcaacaggttttgatggcatcatgtggactttattcatcttaactcatttaaattttctaccacattcccttaagctaatgcaaaagtaccaacaacttaatcttttttttttttttttttttttttttttgagacggagtcttgctctgtcgcccaggctggagtgcagtggcgtgatctcggctcattgcaagccccgcctcccgggttcacaccattctcctgcctcagcctcccgagtagctgggactacaggcatctgctaccacgcccagctaattttctgtatttttagtagagatggggtttcactgtgttagccaggatggtctctatctcctgacctcatgatccactcgcctcagcctcccaaaatgctgggattataggcgtgagccgccacgcccggcccaacaacttaatcttttattagctttgcttaagagggccaattaaatcaaagccctttagttccctttaacagggactggagttatgatgcatgtgttacgacttttggcccactgtccatgcaactctaagtgcaggttgatttgctttccagaaatcccaaaggggctgctcttggatcaccgaagagccttacctatatcaaatcaaaaagacattctgggtcagattagactatgctcctggccctacagattgcacataaactatcataaatacagctttttcagggaactagttctaaaactcttacctgctgagaataagtcttaacactaagatgactgtattatatcaatttattattagaatcagacttatacctagcacaattaactattgtgtgggcaaagaacatttaaagggcatagtagaggtaaggagagacacatactcagctagagataaaaatactaagttgcactgattacttaaaatactagtcacaccataaaagtgccctgtagtttcaaaaacacgtaagcaatgaaatgttagccattatgtgttaaactacttaatctcatttctgtgatgtgaatatttttaaccccctttttgtagatgagggaactgacaagtgacttgtccaaggccatatagctgtgagaaaacccatgcattctcttttcagagttcatgctatcactcaactttaaagtaggccaagattaatgttggtaagggtttgtaatctgtaagaatgctaaaaacgtaagtatatatatcattttagatttgacattttgtatcttgccagtttttaggagaacttttcattttgttaagtatgcatgaatatagttgagtatatgagtaactggttcttatgctgctgttttgtatttttaccagcaggaagattgcaaaagttgatgtatgtaaatcttgaaatatttctaagttttatgtataacaaaatatgtattttaataaacttcttttgatattttaaaaaaaaaaa

SEQ ID NO: 8 - DNA, 2984 bp, Homo sapiens
ggaaattcaaacgtgtttgcggaaaggagtttgggttccatcttttcatttccccagcgcagctttctgtagaaatggaatccgaggatttaagtggcagagaattgacaattgattccataatgaacaaagtgagagacattaaaaataagtttaaaaatgaagaccttactgatgaactaagcttgaataaaatttctgctgatactacagataactcgggaactgttaaccaaattatgatgatggcaaacaacccagaggactggttgagtttgttgctcaaactagagaaaaacagtgttccgctaagtgatgctcttttaaataaattgattggtcgttacagtcaagcaattgaagcgcttcccccagataaatatggccaaaatgagagttttgctagaattcaagtgagatttgctgaattaaaagctattcaagagccagatgatgcacgtgactactttcaaatggccagagcaaactgcaagaaatttgcttttgttcatatatcttttgcacaatttgaactgtcacaaggtaatgtcaaaaaaagtaaacaacttcttcaaaaagctgtagaacgtggagcagtaccactagaaatgctggaaattgccctgcggaatttaaacctccaaaaaaagcagctgctttcagaggaggaaaagaagaatttatcagcatctacggtattaactgcccaagaatcattttccggttcacttgggcatttacagaataggaacaacagttgtgattccagaggacagactactaaagccaggtttttatatggagagaacatgccaccacaagatgcagaaataggttaccggaattcattgagacaaactaacaaaactaaacagtcatgcccatttggaagagtcccagttaaccttctaaatagcccagattgtgatgtgaagacagatgattcagttgtaccttgttttatgaaaagacaaacctctagatcagaatgccgagatttggttgtgcctggatctaaaccaagtggaaatgattcctgtgaattaagaaatttaaagtctgttcaaaatagtcatttcaaggaacctctggtgtcagatgaaaagagttctgaacttattattactgattcaataaccctgaagaataaaacggaatcaagtcttctagctaaattagaagaaactaaagagtatcaagaaccagaggttccagagagtaaccagaaacagtggcaatctaagagaaagtcagagtgtattaaccagaatcctgctgcatcttcaaatcactggcagattccggagttagcccgaaaagttaatacagagcagaaacataccacttttgagcaacctgtcttttcagtttcaaaacagtcaccaccaatatcaacatctaaatggtttgacccaaaatctatttgtaagacaccaagcagcaataccttggatgattacatgagctgttttagaactccagttgtaaagaatgactttccacctgcttgtcagttgtcaacaccttatggccaacctgcctgtttccagcagcaacagcatcaaatacttgccactccacttcaaaatttacaggttttagcatcttcttcagcaaatgaatgcatttcggttaaaggaagaatttattccattttaaagcagataggaagtggaggttcaagcaaggtatttcaggtgttaaatgaaaagaaacagatatatgctataaaatatgtgaacttagaagaagcagataaccaaactcttgatagttaccggaacgaaatagcttatttgaataaactacaacaacacagtgataagatcatccgactttatgattatgaaatcacggaccagtacatctacatggtaatggagtgtggaaatattgatcttaatagttggcttaaaaagaaaaaatccattgatccatgggaacgcaagagttactggaaaaatatgttagaggcagttcacacaatccatcaacatggcattgttcacagtgatcttaaaccagctaactttctgatagttgatggaatgctaaagctaattgattttgggattgcaaaccaaatgcaaccagatacaacaagtgttgttaaagattctcaggttggcacagttaattatatgccaccagaagcaatcaaagatatgtcttcctccagagagaatgggaaatctaagtcaaagataagccccaaaagtgatgtttggtccttaggatgtattttgtactatatgacttacgggaaaacaccatttcagcagataattaatcagatttctaaattacatgccataattgatcctaatcatgaaattgaatttcccgatattccagagaaagatcttcaagatgtgttaaagtgttgtttaaaaagggacccaaaacagaggatatccattcctgagctcctggctcatccctatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcaaataccacctataaaatatattggactgttatactcttgaatccctgtggaaatctacatttgaagacaacatcactctgaagtgttatcagcaaaaaaaattcagtagattatctttaaaagaaaactgtaaaaatagcaaccacttatggtactgtatatattgtagacttgttttctctgttttatgctcttgtgtaatctacttgacatcattttactcttggaatagtgggtggatagcaagtatattctaaaaaactttgtaaataaagttttgtggctaaaatgacactaaaaaaaaaaaaaaaaaa

SEQ ID NO: 9 - DNA, 4543 bp, Homo sapiens
cggcgggcggcgcgcacactgctcgctgggccgcggctcccgggtgtcccaggcccggccggtgcgcagagcatggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatagccaggaggccgagtggctgccggaggagcatgcgcagaggccagttaaagtagatcacctcctcgaacccactccggttccccgcaacccacagctcagctgcccatcccagtccctcgccgtccctcccaggtcgggcagtggaggctgctgtgaaaactctggtacgaatctcatccctcaactgagggccagggacccaggtgtgcctgtgctcctgcccaggagcagcttttggtctccttgggccctttttcccttccctcctttgtttacttatatatatattttttttaaacttaaattttgggtcaacttgacaccactaagatgattttttaaggagctgggggaaggcaggagccttcctttctcctgccccaagggcccagaccctgggcaaacagagctactgagacttggaacctcattgctaccacagacttgcactgaagccggacagctgcccagacacatgggcttgtgacattcgtgaaaaccaaccctgtgggcttatgtctctgccttagggtttgcagagtggaaactcagccgtagggtggcactgggagggggtgggggatctgggcaaggtgggtgattcctcccaggaggtgcttgaggccccgatggactcctgaccataatcctagccccgagacaccatcctgagccagggaacagccccagggttggggggtgccggcatctcccctagctcaccaggcctggcctctgggcagtgtggcctcttggctatttctgtgtccagttttggaggctgagttctggttcatgcagacaaagccctgtccttcagtcttctagaaacagagacaagaaaggcagacacaccgcggccaggcacccatgtgggcgcccaccctgggctccacacagcagtgtcccctgccccagaggtcgcagctaccctcagcctccaatgcattggcctctgtaccgcccggcagccccttctggccggtgctgggttcccactcccggcctaggcacctccccgctctccctgtcacgctcatgtcctgtcctggtcctgatgcccgttgtctaggagacagagccaagcactgctcacgtctctgccgcctgcgtttggaggcccctgggctctcacccagtccccacccgcctgcagagagggaactagggcaccccttgtttctgttgttcccgtgaatttttttcgctatgggaggcagccgaggcctggccaatgcggcccactttcctgagctgtcgctgcctccatggcagcagccagggacccccagaacaagaagaccccgcaggatccctcctgagctcggggggctctgccttctcaggccccgggcttcccttctccccagccagaggtggagccaagtggtccagcgtcactccagtgctcagctgtggctggaggagctggcctgtggcacagccctgagtgtcccaagccgggagccaacgaagccggacacggcttcactgaccagcggctgctcaagccgcaagctctcagcaagtgcccagtggagcctgccgcccccgcctgggcaccgggaccccctcaccatccagtgggcccggagaaacctgatgaacagtttggggactcaggaccagatgtccgtctctcttgcttgaggaatgaagacctttattcacccctgccccgttgcttcccgctgcacatggacagacttcacagcgtctgctcataggacctgcatccttcctggggacgaattccactcgtccaagggacagcccacggtctggaggccgaggaccaccagcaggcaggtggactgactgtgttgggcaagacctcttccctctgggcctgttctcttggctgcaaataaggacagcagctggtgccccacctgcctggtgcattgctgtgtgaatccaggaggcagtggacatcgtaggcagccacggccccgggtccaggagaagtgctccctggaggcacgcaccactgcttcccactggggccggcggggcccacgcacgacgtcagcctcttaccttcccgcctcggctaggggtcctcgggatgccgttctgttccaacctcctgctctgggacgtggacatgcctcaaggatacagggagccggcggcctctcgacggcacgcacttgcctgttggctgctgcggctgtgggcgagcatgggggctgccagcgtctgttgtggaaagtagctgctagtgaaatggctggggccgctggggtccgtcttcacactgcgcaggtctcttctgggcgtctgagctggggtgggagctcctccgcagaaggttggtggggggtccagtctgtgatccttggtgctgtgtgccccactccagcctggggaccccacttcagaaggtaggggccgtgtcccgcggtgctgactgaggcctgcttccccctccccctcctgctgtgctggaattccacagggaccagggccaccgcaggggactgtctcagaagacttgatttttccgtccctttttctccacactccactgacaaacgtccccagcggtttccacttgtgggcttcaggtgttttcaagcacaacccaccacaacaagcaagtgcattttcagtcgttgtgcttttttgttttgtgctaacgtcttactaatttaaagatgctgtcggcaccatgtttatttatttccagtggtcatgctcagccttgctgctctgcgtggcgcaggtgccatgcctgctccctgtctgtgtcccagccacgcagggccatccactgtgacgtcggccgaccaggctggacaccctctgccgagtaatgacgtgtgtggctgggaccttctttattctgtgttaatggctaacctgttacactgggctgggttgggtagggtgttctggcttttttgtggggtttttatttttaaagaaacactcaatcatccta

SEQ ID NO: 10 - DNA, 4086 bp, Homo sapiens
ggccacttggcccgggcctcctttctcctctggtcgtggggaaggagggatgggttggaccttctgcttttctttcaattccctcttttcattctccttcctcctcaatcttcaacacttggctagtcgttaatgccttaagtgcttaatttgttgtgtctggtcctggccagggtctggctgtacaggaggactggaagggcatcctgggagtttcctggtgtccacaggccggacaaaagcaaccccgactccttagagcatggcatggctcagaggtgctggtaaaactgatgggggtttttgctgtccctcccctcagcgccgacaccatgtggatccaggttcggaccatggacgggaggcagacccacacggtggactcgctgtccaggctgaccaaggtggaggagctgaggcggaagatccaggagctgttccacgtggagccaggcctgcagaggctgttctacaggggcaaacagatggaggacggccataccctcttcgactacgaggtccgcctgaatgacaccatccagctcctggtccgccagagcctcgtgctcccccacagcaccaaggagcgggactccgagctctccgacaccgactccggctgctgcctgggccagagtgagtcagacaagtcctccacccacggtgaggcggccgccgagactgacagcaggccagccgatgaggacatgtgggatgagacggaattggggctgtacaaggtcaatgagtacgtcgatgctcgggacacgaacatgggggcgtggtttgaggcgcaggtggtcagggtgacgcggaaggccccctcccgggacgagccctgcagctccacgtccaggccggcgctggaggaggacgtcatttaccacgtgaaatacgacgactacccggagaacggcgtggtccagatgaactccagggacgtccgagcgcgcgcccgcaccatcatcaagtggcaggacctggaggtgggccaggtggtcatgctcaactacaaccccgacaaccccaaggagcggggcttctggtacgacgcggagatctccaggaagcgcgagaccaggacggcgcgggaactctacgccaacgtggtgctgggggatgattctctgaacgactgtcggatcatcttcgtggacgaagtcttcaagattgagcggccgggtgaagggagccccatggttgacaaccccatgagacggaagagcgggccgtcctgcaagcactgcaaggacgacgtgaacagactctgccgggtctgcgcctgccacctgtgcgggggccggcaggaccccgacaagcagctcatgtgcgatgagtgcgacatggccttccacatctactgcctggacccgcccctcagcagtgttcccagcgaggacgagtggtactgccctgagtgccggaatgatgccagcgaggtggtactggcgggagagcggctgagagagagcaagaagaaggcgaagatggcctcggccacatcgtcctcacagcgggactggggcaagggcatggcctgtgtgggccgcaccaaggaatgtaccatcgtcccgtccaaccactacggacccatcccggggatccccgtgggcaccatgtggcggttccgagtccaggtcagcgagtcgggtgtccatcggccccacgtggctggcatacacggccggagcaacgacggagcgtactccctagtcctggcggggggctatgaggatgatgtggaccatgggaattttttcacatacacgggtagtggtggtcgagatctttccggcaacaagaggaccgcggaacagtcttgtgatcagaaactcaccaacaccaacagggcgctggctctcaactgctttgctcccatcaatgaccaagaaggggccgaggccaaggactggcggtcggggaagccggtcagggtggtgcgcaatgtcaagggtggcaagaatagcaagtacgcccccgctgagggcaaccgctacgatggcatctacaaggttgtgaaatactggcccgagaaggggaagtccgggtttctcgtgtggcgctaccttctgcggagggacgatgatgagcctggcccttggacgaaggaggggaaggaccggatcaagaagctggggctgaccatgcagtatccagaaggctacctggaagccctggccaaccgagagcgagagaaggagaacagcaagagggaggaggaggagcagcaggaggggggcttcgcgtcccccaggacgggcaagggcaagtggaagcggaagtcggcaggaggtggcccgagcagggccgggtccccgcgccggacatccaagaaaaccaaggtggagccctacagtctcacggcccagcagagcagcctcatcagagaggacaagagcaacgccaagctgtggaatgaggtcctggcgtcactcaaggaccggccggcgagcggcagcccgttccagttgttcctgagtaaagtggaggagacgttccagtgtatctgctgtcaggagctggtgttccggcccatcacgaccgtgtgccagcacaacgtgtgcaaggactgcctggacagatcctttcgggcacaggtgttcagctgccctgcctgccgctacgacctgggccgcagctatgccatgcaggtgaaccagcctctgcagaccgtcctcaaccagctcttccccggctacggcaatggccggtgatctccaagcacttctcgacaggcgttttgctgaaaacgtgtcggagggctcgttcatcggcactgattttgttcttagtgggcttaacttaaacaggtagtgtttcctccgttccctaaaaaggtttgtcttcctttttttttttatttttatttttcaaatctatacattttcaggaatttatgtattctggctaaaagttggacttctcagtattgtgtttagttctttgaaaacataaaagcctgcaatttctcgacaaaacaacacaagattttttaaagatggaatcagaaactacgtggtgtggaggctgttgatgtttctggtgtcaagttctcagaagttgctgccaccaactctttaagaaggcgacaggatcagtccttctctcgggttctggcccccaaggtcagagcaagcatcttcctgacagcattttgtcatctaaagtccagtgacatggttccccgtggtggcccgtggcagcccgtggcatggcgtggctcagctgtctgttgaagttgttgcaaggaaaagaggaaacatctcgggcctagttcaaacctttgcctcaaagccatcccccaccagactgcttagcgtctgagatccgcgtgaaaagtcctctgcccacgagagcagggagttggggccacgcagaaatggcctcaaggggactctgctccacgtggggccaggcgtgtgactgacgctgtccgacgaaggcggccacggacggacgccagcacacgaagtcacgtgcaagtgcctttgattcgttccttctttctaaagacgacagtctttgttgttagcactgaattattgaaaatgtcaaccagattctagaaactgcggtcatccagttcttcctgacaccggatgggtgcttgggaaccgtttgagccttatagatcatttacattcaatttttttaactcagcaagtgagaacttacaagagggtttttttaaaatttttttttctcttaatgaacacattttctaaatgaattttttttgtagttactgtatatgtaccaagaaagatataacgttagggtttggttgtttttgtttttgtattttttttcttttgaaagggtttgttaatttttctaattttaccaaagtttgcagcctatacctcaataaaacagggatattttaaatcacatacctgcagacaaactggagcaatgttatttttaaagggtttttttcacctccttattcttagattattaatgtattagggaagaatgagacaattttgtgtaggctttttctaaagtccagtactttgtccagattttagattctcagaataaatgtttttcacagatagaaaaaaaaaaaaaaaaaaaaaa

SEQ ID NO: 11 - protein, 16 aa, Homo sapiens
RPSTSRSLYASSPGGV

SEQ ID NO: 12 - protein, 21 aa, Homo sapiens
FADLSEAANRNNDALRQAKQE

SEQ ID NO: 13 - protein, 16 aa, Homo sapiens
VDALKGTNESLERQMR